SEQUENCE LISTING (80 sequences)

SEQ ID NO: 1 - protein, 52 aa, Artificial Sequence
  Synthetic Construct
LQSRPEPTAPPEESFRFGVETTTPPQKQEPIDKELYPLTSLRSLFGNDPSSQ

SEQ ID NO: 2 - protein, 63 aa, Artificial Sequence
  Synthetic Construct
PMAQVHQGLMPTAPPEDPAVDLLKNYMQLGKQQREKQRESREKPYKEVTEDLLHLNSLFGGDQ

SEQ ID NO: 3 - protein, 7 aa, Artificial Sequence
  Synthetic Construct
  VARIANT 2, 4, 5: Xaa can be any amino acid
  VARIANT (4)...(5): Xaa can be any naturally occurring amino acid
DXAXXLL

SEQ ID NO: 4 - protein, 7 aa, Artificial Sequence
  Synthetic Construct
DPAVDLL

SEQ ID NO: 5 - protein, 10 aa, Artificial Sequence
  Synthetic Construct
DPAVDLLKNY

SEQ ID NO: 6 - protein, 52 aa, Artificial Sequence
  Synthetic Construct
LQSRPEPTAPPEESDPAVDLLTTPPQKQEPIDKELYPLTSLRSLFGNDPSSQ

SEQ ID NO: 7 - protein, 16 aa, Artificial Sequence
  Synthetic Construct
  VARIANT 6, 9, 14: Xaa can be Val, Ile, Leu, Phe, Trp, Tyr or Met
  VARIANT 2, 3, 4, 5, 7, 8, 10, 11, 12, 15: Xaa can be any amino acid
WXXXXXXXXXXXAXXH

SEQ ID NO: 8 - protein, 12 aa, Artificial Sequence
  Synthetic Construct
ESKYGPPCPPCP

SEQ ID NO: 9 - DNA, 36 bp, Artificial Sequence
  Synthetic Construct
gaatctaagtacggaccgccctgccccccttgccct

SEQ ID NO: 10 - protein, 119 aa, Artificial Sequence
  Synthetic Construct
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Artificial Sequence
  Synthetic Construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 282 aa, Artificial Sequence
  Synthetic Construct
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 13 - protein, 24 aa, Artificial Sequence
  Synthetic Construct
LEGGGEGRGSLLTCGDVEENPGPR

SEQ ID NO: 14 - protein, 357 aa, Artificial Sequence
  Synthetic Construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 15 - protein, 27 aa, Homo sapiens
  Synthetic Construct
FWVLVVVGGVLACYSLLVTVAFIIFWV

SEQ ID NO: 16 - protein, 66 aa, Homo sapiens
  Synthetic Construct
IEVMYPPPYLDNEKSNGTIIHVKGKHLCPSPLFPGPSKPFWVLVVVGGVLACYSLLVTVAFIIFWV

SEQ ID NO: 17 - protein, 41 aa, Homo sapiens
  Synthetic Construct
RSKRSRLLHSDYMNMTPRRPGPTRKHYQPYAPPRDFAAYRS

SEQ ID NO: 18 - protein, 41 aa, Artificial Sequence
  Synthetic Construct
RSKRSRGGHSDYMNMTPRRPGPTRKHYQPYAPPRDFAAYRS

SEQ ID NO: 19 - protein, 42 aa, Homo sapiens
  Synthetic Construct
KRGRKKLLYIFKQPFMRPVQTTQEEDGCSCRFPEEEEGGCEL

SEQ ID NO: 20 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 112 aa, Artificial Sequence
  Synthetic Construct
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 112 aa, Artificial Sequence
  Synthetic Construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 335 aa, Artificial Sequence
  Synthetic Construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 24 - protein, 18 aa, Artificial Sequence
  Synthetic Construct
EGRGSLLTCGDVEENPGP

SEQ ID NO: 25 - protein, 22 aa, Artificial Sequence
  Synthetic Construct
GSGATNFSLLKQAGDVEENPGP

SEQ ID NO: 26 - protein, 19 aa, Artificial Sequence
  Synthetic Construct
ATNFSLLKQAGDVEENPGP

SEQ ID NO: 27 - protein, 20 aa, Artificial Sequence
  Synthetic Construct
QCTNYALLKLAGDVESNPGP

SEQ ID NO: 28 - protein, 22 aa, Artificial Sequence
  Synthetic Construct
VKQTLNFDLLKLAGDVESNPGP

SEQ ID NO: 29 - protein, 30 aa, Artificial Sequence
  Synthetic Construct
PGGGSGGGGSGGGGSGGGGSGGGGSGGGGP

SEQ ID NO: 30 - protein, 17 aa, Artificial Sequence
  Synthetic Construct
GSADDAKKDAAKKDGKS

SEQ ID NO: 31 - DNA, 66 bp, Artificial Sequence
  Synthetic Construct
atgcttctcctggtgacaagccttctgctctgtgagttaccacacccagcattcctcctgatccca

SEQ ID NO: 32 - protein, 22 aa, Artificial Sequence
  Synthetic Construct
MLLLVTSLLLCELPHPAFLLIP

SEQ ID NO: 33 - protein, 18 aa, Artificial Sequence
  Synthetic Construct
MALPVTALLLPLALLLHA

SEQ ID NO: 34 - protein, 159 aa, Streptomyces avidinii
  UniProt No. P22629
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 35 - protein, 126 aa, Streptomyces avidinii
  Synthetic Construct
EAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 36 - protein, 126 aa, Streptomyces avidinii
  Synthetic Construct
EAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 37 - protein, 8 aa, Artificial Sequence
  Synthetic Construct
WSHPQFEK

SEQ ID NO: 38 - protein, 28 aa, Artificial Sequence
  Synthetic Construct
WSHPQFEKGGGSGGGSGGGSWSHPQFEK

SEQ ID NO: 39 - protein, 24 aa, Artificial Sequence
  Synthetic Construct
WSHPQFEKGGGSGGGSWSHPQFEK

SEQ ID NO: 40 - protein, 28 aa, Artificial Sequence
  Synthetic Construct
WSHPQFEKGGGSGGGSGGSAWSHPQFEK

SEQ ID NO: 41 - protein, 127 aa, Streptomyces avidinii
  Synthetic Construct
MEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 42 - protein, 8 aa, Artificial Sequence
  Synthetic Construct
  VARIANT 2: Xaa is any amino acid
  VARIANT 7: Xaa is Gly or Glu
  VARIANT 8: Xaa is Gly, Lys or Arg
WXHPQFXX

SEQ ID NO: 43 - protein, 8 aa, Artificial Sequence
  Synthetic Construct
WRHPQFGG

SEQ ID NO: 44 - protein, 17 aa, Artificial Sequence
  Synthetic Construct
  VARIANT 9: Xaa us any amino acid
  REPEAT 9: Repeated 8 or 12 times
WSHPQFEKXWSHPQFEK

SEQ ID NO: 45 - protein, 20 aa, Artificial Sequence
  Synthetic Construct
  REPEAT (9)...(12): Repeated 2 or 3 times
WSHPQFEKGGGSWSHPQFEK

SEQ ID NO: 46 - protein, 30 aa, Artificial Sequence
  Synthetic Construct
SAWSHPQFEKGGGSGGGSGGGSWSHPQFEK

SEQ ID NO: 47 - protein, 30 aa, Artificial Sequence
  Synthetic Construct
SAWSHPQFEKGGGSGGGSGGSAWSHPQFEK

SEQ ID NO: 48 - protein, 159 aa, Streptomyces avidinii
  Synthetic Construct
DPSKDSKAQVSAAEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 49 - protein, 126 aa, Streptomyces avidinii
  Synthetic Construct
EAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 50 - protein, 127 aa, Streptomyces avidinii
  Synthetic Construct
MEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 51 - protein, 4 aa, Artificial Sequence
  Synthetic Construct
HPQF

SEQ ID NO: 52 - protein, 8 aa, Artificial Sequence
  Synthetic Construct
  VARIANT 1: Xaa is Trp, Lys or Arg
  VARIANT 2: Xaa is any amino acid
  VARIANT 7: Xaa is Gly or Glu
  VARIANT 8: Xaa is Gly, Lys or Arg
XXHPQFXX

SEQ ID NO: 53 - protein, 159 aa, Streptomyces avidinii
  Synthetic Construct
DPSKDSKAQVSAAEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 54 - protein, 126 aa, Streptomyces avidinii
  Synthetic Construct
EAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 55 - protein, 139 aa, Streptomyces avidinii
  Synthetic Construct
DPSKDSKAQVSAAEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 56 - protein, 127 aa, Streptomyces avidinii
  Synthetic Construct
MEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 57 - protein, 11 aa, Artificial Sequence
  CDR L1
RASQDISKYLN

SEQ ID NO: 58 - protein, 7 aa, Artificial Sequence
  CDR L2
SRLHSGV

SEQ ID NO: 59 - protein, 9 aa, Artificial Sequence
  CDR L3
GNTLPYTFG

SEQ ID NO: 60 - protein, 5 aa, Artificial Sequence
  CDR H1
DYGVS

SEQ ID NO: 61 - protein, 16 aa, Artificial Sequence
  CDR H2
VIWGSETTYYNSALKS

SEQ ID NO: 62 - protein, 7 aa, Artificial Sequence
  CDR H3
YAMDYWG

SEQ ID NO: 63 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 64 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 65 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 66 - protein, 11 aa, Artificial Sequence
  CDR L1
KASQNVGTNVA

SEQ ID NO: 67 - protein, 7 aa, Artificial Sequence
  CDR L2
SATYRNS

SEQ ID NO: 68 - protein, 9 aa, Artificial Sequence
  CDR L3
QQYNRYPYT

SEQ ID NO: 69 - protein, 5 aa, Artificial Sequence
  CDR H1
SYWMN

SEQ ID NO: 70 - protein, 17 aa, Artificial Sequence
  CDR H2
QIYPGDGDTNYNGKFKG

SEQ ID NO: 71 - protein, 13 aa, Artificial Sequence
  CDR H3
KTISSVVDFYFDY

SEQ ID NO: 72 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 73 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 74 - protein, 15 aa, Artificial Sequence
  Linker
GGGGSGGGGSGGGGS

SEQ ID NO: 75 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 76 - protein, 12 aa, Artificial Sequence
  HC-CDR3
HYYYGGSYAMDY

SEQ ID NO: 77 - protein, 7 aa, Artificial Sequence
  LC-CDR2
HTSRLHS

SEQ ID NO: 78 - protein, 9 aa, Artificial Sequence
  LC-CDR3
QQGNTLPYT

SEQ ID NO: 79 - DNA, 735 bp, Artificial Sequence
  scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 80 - protein, 18 aa, Artificial Sequence
  Linker
GSTSGSGKPGSGEGSTKG

(19) United States
(12) Patent Application Publication
(10) Pub. No.: US 2023/0090117 A1
(43) Pub. Date: Mar. 23, 2023

(54) METHODS FOR T CELL TRANSDUCTION

(71) Applicant: Juno Therapeutics, Inc., Seattle, WA (US)

(72) Inventors: Neil HAIG, Seattle, WA (US); Jeffrey TEOH, Seattle, WA (US)

(73) Assignee: Juno Therapeutics, Inc., Seattle, WA (US)

(21) Appl. No.: 17/796,224

(22) PCT Filed: Jan. 27, 2021
(86) PCT No.: PCT/US2021/015333

§ 371 Date: Jul. 28, 2022

Related U.S. Application Data
(60) Provisional application No. 62/967,005, filed on Jan. 28, 2020.

(51) Int. Cl. C12N 5/0783; C12N 15/62; C12N 15/86

(52) U.S. Cl. CPC C12N 5/0636; C12N 15/625; C12N 15/86; C12N 2760/20222; C12N 2760/20223; C12N 2501/51; C12N 2740/15043

(57) ABSTRACT

Provided herein are methods for transduction of T cells. In some embodiments, the provided methods include transduction of T cells by incubation with a retroviral vector particle, e.g., lentiviral vector, in which the cells have been selected for CCR7+ expression. The provided methods improve the process for genetically engineering T cells by increasing transduction frequency and/or by reducing the variability in transduction frequency among biological samples. Also provided are resulting cells transduced with a recombinant or heterologous gene, and compositions thereof. In some embodiments, the provided cells and compositions can be used in methods of adoptive immunotherapy

### CROSS-REFERENCE TO RELATED APPLICATIONS

This application claims priority to U.S. provisional application 62/967,005, filed Jan. 28, 2020, entitled “METHODS FOR T CELL TRANSDUCTION,” the contents of which are incorporated by reference in their entirety for all purposes.

### INCORPORATION BY REFERENCE OF SEQUENCE LISTING

The content of the following submission on ASCII text file is incorporated herein by reference in its entirety: a computer readable form (CRF) of the Sequence Listing is provided as a file entitled 73504_2023140_SEQLIST.txt, created Jan. 27, 2021, which is 53,345 bytes in size.

### FIELD

The present disclosure provides methods for transduction of T cells. In some embodiments, the provided methods include transduction of T cells by incubation with a retroviral vector particle, e.g. lentiviral vector, in which the cells have been selected for CCR7+ expression. In some embodiments, such methods result in improving the process for genetically engineering T cells by increasing transduction frequency and/or by reducing the variability in transduction frequency among biological samples. Also provided are resulting cells, transduced with a recombinant or heterologous gene, such as one encoding a chimeric receptor such as a chimeric antigen receptor, or other recombinant antigen receptor such as a transgenic T cell receptor, and compositions thereof. In some embodiments, the provided cells and compositions can be used in methods of adoptive immunotherapy.

### BACKGROUND

Various strategies are available for transducing T cell populations in vitro, including for transducing T cells in vitro for use in adoptive cellular immunotherapy or cancer therapy. Improved strategies are needed for transducing cell populations in vitro, including for research, diagnostic and therapeutic purposes, such that transduction frequency is increased and is more consistent among biological samples. Provided are methods that meet such needs.

### SUMMARY

Provided herein is a method for increasing transduction frequency of primary T cells, the method comprising: (a) selecting primary T cells that are positive for surface expression of CCR7 from a biological sample comprising a population of primary T cells, thereby generating an input population enriched in CCR7+ primary T cells; (b) optionally, incubating the input population under stimulatory conditions, thereby generating a stimulated composition, wherein said stimulating conditions comprise the presence of a stimulatory reagent capable of activating one or more intracellular signaling domains of one or more components of a TCR complex and/or one or more intracellular signaling domains of one or more costimulatory molecules; and (c) a viral vector particle comprising a heterologous polynucleotide encoding a recombinant protein with T cells of the input population of cells, or optionally of the stimulated composition, thereby generating a population of transduced cells.

Also provided herein is a method for increasing transduction frequency of primary T cells, the method comprising: (a) selecting primary T cells that are positive for surface expression of CCR7 from a biological sample comprising a population of primary T cells, thereby generating an input population enriched in CCR7+ primary T cells; (b) incubating the input population under stimulatory conditions, thereby generating a stimulated composition, wherein said stimulating conditions comprise the presence of a stimulatory reagent capable of activating one or more intracellular signaling domains of one or more components of a TCR complex and/or one or more intracellular signaling domains of one or more costimulatory molecules; and (c) incubating a viral vector particle comprising a heterologous polynucleotide encoding a recombinant protein with T cells of the stimulated composition, thereby generating a population of transduced cells.

Also provided herein is a method for increasing transduction frequency of primary T cells, the method comprising: (a) selecting primary T cells that are positive for surface expression of CCR7 from a biological sample comprising a population of primary T cells, thereby generating an input population enriched in CCR7+ primary T cells; and (b) incubating a viral vector particle comprising a heterologous polynucleotide encoding a recombinant protein with T cells of the input population of cells, thereby generating a population of transduced cells.

Also provided herein is a method for increasing transduction frequency of primary T cells, the method comprising: (a) incubating an input population of primary T cells enriched in CCR7+ T cells under stimulatory conditions, thereby generating a stimulated composition, wherein said stimulating conditions comprise the presence of a stimulatory reagent capable of activating one or more intracellular signaling domains of one or more components of a TCR complex and/or one or more intracellular signaling domains of one or more costimulatory molecules; and (b) incubating a viral vector particle comprising a heterologous polynucleotide encoding a recombinant protein with T cells of the stimulated composition, thereby generating a population of transduced cells.

Also provided herein is a method for increasing transduction frequency of primary T cells, the method comprising incubating a viral vector particle comprising a heterologous polynucleotide encoding a recombinant protein with T cells of an input population of primary T cells enriched in CCR7+ T cells, thereby generating a population of transduced cells.

In some of any such embodiments, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the input population are CCR7+ primary T cells. In some of any such embodiments, at least 90%, at least 91%, at least 92%, at least 93%, at least 94%, at least 95%, at least 96%, at least 97%, at least 98%, at least 99%, or 100% of the input population are CCR7+ primary T cells. In some of any such embodiments, at least 95%, at least 96%, at least 97%, at least 98%, at least 99%, or 100% of the input population are CCR7+ primary T cells.

In some of any such embodiments, the selecting does not comprise selecting for cells that are (a) CCR7+ and CD45RO+; or (b) CCR7+ and CD27+; or (c) CCR7+ and CD45RA−; or (d) CCR7+ and CD62L+; or (e) CCR7+ and CD45RA+; or (f) CCR7+ and CD62L−. In some of any such embodiments, the input population is not enriched in T cells that are (a) CCR7+ and CD45RO+; or (b) CCR7+ and CD27+; or (c) CCR7+ and CD45RA−; or (d) CCR7+ and CD62L+; or (e) CCR7+ and CD45RA+; or (f) CCR7+ and CD62L−. In some of any such embodiments, the input population is not enriched in CCR7+ and CD45RO+ T cells, optionally wherein less than 85% of the total cells of the input population are CCR7+ and CD45RO+ T cells. In some of any such embodiments, the input population is not enriched in CCR7+ and CD27+ T cells, optionally wherein less than 85% of the total cells of the input population are CCR7+ and CD27+ T cells. In some of any such embodiments, the input population is not enriched in CCR7+ and CD45RA− T cells, optionally wherein less than 85% of the total cells of the input population are CCR7+ and CD45RA− T cells. In some of any such embodiments, the input population is not enriched in CCR7+ and CD62L+ T cells, optionally wherein less than 85% of the total cells of the input population are CCR7+ and CD62L+ T cells. In some of any such embodiments, the input population is not enriched in CCR7+ and CD45RA+ T cells, optionally wherein less than 85% of the total cells of the input population are CCR7+ and CD45RA+ T cells. In some of any such embodiments, the input population is not enriched in CCR7+ and CD62L− T cells, optionally wherein less than 85% of the total cells of the input population are CCR7+ and CD62L− T cells.

In some of any such embodiments, the biological sample is a blood sample. In some of any such embodiments, the biological sample is a leukapheresis sample.

In some of any such embodiments, the T cells are unfractionated T cells, are enriched or isolated CD3+ T cells, are enriched or isolated CD4+ T cells, or are enriched or isolated CD8+ T cells.

In some of any such embodiments, the input population comprises at least 80%, at least 85%, at least 90%, or at least 95% cells that are CD4+ T cells or CD8+ T cells. In some of any such embodiments, the input population comprises at least 80%, at least 85%, at least 90%, or at least 95% cells that are CD4+ T cells. In some of any such embodiments, the input population comprises at least 80%, at least 85%, at least 90%, or at least 95% cells that are CD8+ T cells. In some of any such embodiments, the input population comprises at least 80%, at least 85%, at least 90%, or at least 95% cells that are CD4+ T cells and CD8+ T cells. In some of any such embodiments, the ratio of the CD4+ T cells to the CD8+ T cells is or is about 1:1, 1:2, 2:1, 1:3, or 3:1. In some of any such embodiments, the input population comprises at least 80%, at least 85%, at least 90%, or at least 95% cells that are CD3+ T cells.

In some of any such embodiments, the input population comprises between 100×10⁶ and 500×10⁶ total T cells. In some of any such embodiments, the input population comprises between 200×10⁶ and 400×10⁶ total T cells, optionally at or about 300×10⁶ total T cells. In some of any such embodiments, the total T cells are viable T cells.

In some of any such embodiments, at least 10%, at least 20%, at least 30%, at least 40%, at least 50%, or at least 60% of the cells of the stimulated composition: (i) express a surface marker selected from the group consisting of HLA-DR, CD25, CD69, CD71, CD40L and 4-1BB; (ii) comprise intracellular expression of a cytokine selected from the group consisting of IL-2, IFN-gamma, TNF-alpha; (iii) are in the G1 or later phase of the cell cycle; and/or (iv) are capable of proliferating.

In some of any such embodiments, the stimulatory reagent comprises a primary agent that specifically binds to a member of a TCR complex, optionally that specifically binds to CD3. In some of any such embodiments, the stimulatory reagent further comprises a secondary agent that specifically binds to a T cell costimulatory molecule, optionally wherein the costimulatory molecule is selected from CD28, CD137 (4-1-BB), OX40, or ICOS. In some of any such embodiments, the primary and/or secondary agents comprise an antibody, optionally wherein the stimulatory reagent comprises incubation with an anti-CD3 antibody and an anti-CD28 antibody, or an antigen-binding fragment thereof.

In some of any such embodiments, the primary agent and/or secondary agent are present on the surface of a solid support. In some of any such embodiments, the solid support is or comprises a bead. In some of any such embodiments, the primary agent and secondary agent are reversibly bound on the surface of an oligomeric particle reagent comprising a plurality of streptavidin or streptavidin mutein molecules. In some of any such embodiments, each of the plurality of the streptavidin or streptavidin mutein molecules comprise the amino acid sequence of Val⁴⁴-Thr⁴⁵-Ala⁴⁶-Arg⁴⁷ or Ile⁴⁴-Gly⁴⁵-Ala⁴⁶-Arg⁴⁷ at sequence positions corresponding to positions 44 to 47 with reference to positions in streptavidin in the sequence of amino acids set forth in SEQ ID NO: 34. In some of any such embodiments, each of the plurality of the streptavidin or streptavidin mutein molecules is or comprises: a) the sequence of amino acids set forth in SEQ ID NO: 35 or 56; or b) a sequence of amino acids that exhibit at least 85%, 86%, 87%, 88%, 89%, 89%, 90%, 91%, 92%, 93%, 94%, 95%, 96%, 97%, 98%, or 99% or more sequence identity to the sequence of amino acids set forth in SEQ ID NO: 35 or 56; or c) a functional fragment of a) or b) that reversibly binds to biotin, a biotin analog, or a streptavidin-binding peptide. In some of any such embodiments, each of the plurality of the streptavidin or streptavidin mutein molecules are streptavidin mutein molecules, and wherein each of the plurality of the streptavidin mutein molecules is or comprises: a) the sequence of amino acids set forth in any of SEQ ID NOS: 36, 41, 48-50, or 53-55; b) a sequence of amino acids that exhibit at least 85%, 86%, 87%, 88%, 89%, 89%, 90%, 91%, 92%, 93%, 94%, 95%, 96%, 97%, 98%, or 99% or more sequence identity to any of SEQ ID NOS: 36, 41, 48-50, or 53-55 and contain the amino acid sequence corresponding to Val44-Thr45-Ala46-Arg47 or Ile44-Gly45-Ala46-Arg47 and/or reversibly bind to biotin, a biotin analog or a streptavidin-binding peptide; or c) a functional fragment of a) or b) that reversibly binds to biotin, a biotin analog, or a streptavidin-binding peptide, optionally wherein each of the plurality of the streptavidin mutein molecules is or comprises the amino acid sequence set forth in SEQ ID NO:36 or SEQ ID NO:41.

In some of any such embodiments, the population of transduced cells comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% cells that express the recombinant protein. In some of any such embodiments, the population of transduced cells comprises at least 55%, at least 60%, at least 65%, at least 70%, at least 75%, at least 80%, at least 85%, at least 90%, or at least 95% cells that express the recombinant protein.

In some of any such embodiments, the percentage of cells in the population of transduced cells expressing the recombinant protein is at least 0.5-fold, at least 1-fold, at least 1.5-fold, or at least 2-fold greater as compared to a cell composition that was not enriched for CCR7+ primary T cells through a selection step.

In some of any such embodiments, the incubating the viral vector particle comprises a step of spinoculating the viral vector particles with the input population. In some of any such embodiments, the incubating the viral vector particle comprises a step of spinoculating the viral vector particles with the stimulated composition.

In some of any such embodiments, spinoculating comprises rotating, in an internal cavity of a centrifugal chamber, the viral vector particles and the input population, wherein the rotation is at a relative centrifugal force at an internal surface of the side wall of the cavity that is: between or between about 500 g and 2500 g, 500 g and 2000 g, 500 g and 1600 g, 500 g an 1000 g, 600 g and 1600 g, 600 g and 1000 g, 1000 g and 2000 g, or 1000 g and 1600 g, each inclusive; or at least or at least about 600 g, 800 g, 1000 g, 1200 g, 1600 g, or 2000 g. In some of any such embodiments, spinoculating comprises rotating, in an internal cavity of a centrifugal chamber, the viral vector particles and the stimulated composition, wherein the rotation is at a relative centrifugal force at an internal surface of the side wall of the cavity that is: between or between about 500 g and 2500 g, 500 g and 2000 g, 500 g and 1600 g, 500 g an 1000 g, 600 g and 1600 g, 600 g and 1000 g, 1000 g and 2000 g, or 1000 g and 1600 g, each inclusive; or at least or at least about 600 g, 800 g, 1000 g, 1200 g, 1600 g, or 2000 g.

In some of any such embodiments, spinoculating is for a time that is: greater than or about 5 minutes, greater than or about 10 minutes, greater than or about 15 minutes, greater than or about 20 minutes, greater than or about 30 minutes, greater than or about 45 minutes, greater than or about 60 minutes, greater than or about 90 minutes, or greater than or about 120 minutes; or between or between about 5 minutes and 60 minutes, 10 minutes and 60 minutes, 15 minutes and 60 minutes, 15 minutes and 45 minutes, 30 minutes and 60 minutes, or 45 minutes and 60 minutes, each inclusive.

In some of any such embodiments, the method further comprises, during at least a portion of the incubating, contacting the input population and/or viral vector particles with a transduction adjuvant. In some of any such embodiments, the method further comprises, during at least a portion of the incubating, contacting the stimulated composition and/or viral vector particles with a transduction adjuvant. In some of any such embodiments, the method further comprises, during at least a portion of the incubating, contacting the stimulated composition and/or viral vector particles with a transduction adjuvant.

In some of any such embodiments, the method further comprises, during at least a portion of the incubating, contacting the input population and/or viral vector particles with a transduction adjuvant.

In some of any such embodiments, the contacting is carried out prior to, concomitant with, or after the spinoculating the viral vector particles with the input population. In some of any such embodiments, the contacting is carried out prior to, concomitant with, or after the spinoculating the viral vector particles with the stimulated composition.

In some of any such embodiments, at least a portion of the incubation of the viral vector particle is carried out at or about 37° C.±2° C. In some of any such embodiments, at least a portion of the incubation of the viral vector particle is carried out after the spinoculation. In some of any such embodiments, the at least a portion of the incubation of the viral vector particle is carried out for no more than or no more than about 2 hours, 4 hours, 12 hours, 18 hours, 24 hours, 30 hours, 36 hours, 48 hours, 60 hours, or 72 hours. In some of any such embodiments, the at least a portion of the incubation of the viral vector particle is carried out for or for about 24 hours. In some of any such embodiments, the total duration of the incubation of the viral vector particle is for no more than 12 hours, 24 hours, 36 hours, 48 hours, or 72 hours.

In some of any such embodiments, the viral vector particle is a lentiviral vector particle. In some of any such embodiments, the lentiviral vector particle is replication defective. In some of any such embodiments, the viral vector particle is pseudotyped with a viral envelope glycoprotein. In some of any such embodiments, the viral envelope glycoprotein is VSV-G.

In some of any such embodiments, the viral vector particle is incubated at a multiplicity of infection of less than or less than about 20.0 or less than or less than about 10.0. In some of any such embodiments, the viral vector particle is incubated at a multiplicity of infection from or from about 1.0 IU/cell to 10 IU/cell, or 2.0 U/cell to 5.0 IU/cell; or the viral vector particle is incubated at a multiplicity of infection of at least or at least about 1.6 IU/cell, 1.8 IU/cell, 2.0 IU/cell, 2.4 IU/cell, 2.8 IU/cell, 3.2 IU/cell, 3.6 IU/cell, 4.0 IU/cell, 5.0 IU/cell, 6.0 IU/cell, 7.0 IU/cell, 8.0 IU/cell, 9.0 IU/cell, or 10.0 IU/cell.

In some of any such embodiments, the stimulated composition comprises at least at or about at least or about 50×10⁶ cells, 100×10⁶ cells, or 200×10⁶ cells. In some of any such embodiments, the stimulated composition comprises between at or about 50×10⁶ cells and at or about 300×10⁶ cells, inclusive. In some of any such embodiments, the stimulated composition comprises between at or about 100×10⁶ cells and at or about 200×10⁶ cells, inclusive.

In some of any such embodiments, the input population comprises at least at or about at least or about 50×10⁶ cells, 100×10⁶ cells, or 200×10⁶ cells. In some of any such embodiments, the input population comprises between at or about 50×10⁶ cells and at or about 300×10⁶ cells, inclusive. In some of any such embodiments, the input population comprises between at or about 100×10⁶ cells and at or about 200×10⁶ cells, inclusive.

In some of any such embodiments, the T cells incubated with the viral particle comprises at least at or about at least or about 50×10⁶ cells, 100×10⁶ cells, or 200×10⁶ cells. In some of any such embodiments, the T cells incubated with the viral particle comprises between at or about 50×10⁶ cells and at or about 300×10⁶ cells, inclusive. In some of any such embodiments, the T cells incubated with the viral particle comprises between at or about 100×10⁶ cells and at or about 200×10⁶ cells, inclusive

In some of any such embodiments, the recombinant protein is an antigen receptor. In some of any such embodiments, the antigen receptor is a transgenic T cell receptor (TCR). In some of any such embodiments, the antigen receptor is a chimeric antigen receptor (CAR). In some of any such embodiments, the CAR comprises an extracellular antigen-recognition domain that specifically binds to a target antigen and an intracellular signaling domain comprising an ITAM. In some of any such embodiments, the CAR comprises an extracellular antigen-recognition domain that specifically binds to a target antigen, an intracellular signaling domain comprising an ITAM, and a transmembrane domain linking the extracellular domain and the intracellular signaling domain. In some of any such embodiments, the intracellular signaling domain comprises an intracellular domain of a CD3-zeta (CD3ζ) chain. In some of any such embodiments, the CAR further comprises a transmembrane domain linking the extracellular domain and the intracellular signaling domain. In some of any such embodiments, the transmembrane domain comprises a transmembrane portion of CD28. In some of any such embodiments, the intracellular signaling domain further comprises an intracellular signaling domain of a T cell costimulatory molecule. In some of any such embodiments, the T cell costimulatory molecule is selected from the group consisting of CD28 and 41BB.

In some of any such embodiments, the antigen receptor specifically binds to an antigen associated with a disease or condition or specifically binds to a universal tag. In some of any such embodiments, the disease or condition is a cancer, an autoimmune disease or disorder, or an infectious disease.

In some of any such embodiments, the population of transduced cells comprises T cells transduced with the heterologous polynucleotide.

In some of any such embodiments, at least 20%, at least 25%, at least 30%, at least 35%, at least 40%, at least 45%, at least 50%, at least 55%, at least 60%, at least 65%, at least 70%, at least 75%, at least 80%, or at least 85% of the T cells in the population of transduced cells are transduced with the heterologous polynucleotide. In some of any such embodiments, at least 55%, at least 60%, at least 65%, at least 70%, at least 75%, at least 80%, or at least 85% of the T cells in the population of transduced cells are transduced with the heterologous polynucleotide. In some of any such embodiments, at least 95%, at least 96%, at least 97%, at least 98%, or at least 99% of the T cells transduced with the heterologous polynucleotide are CCR7+.

In some of any such embodiments, the method further comprises recovering or isolating from the population of transduced cells the transduced T cells produced by the method.

In some of any such embodiments, among a plurality of populations of transduced cells, the percentage of T cells in the population of transduced cells that are transduced with the heterologous polynucleotide varies by 30% or less, 25% or less, 20% or less, 15% or less, or 10% or less.

In some of any such embodiments, the method is carried out in vitro or ex vivo.

Also provided herein is a composition comprising a population of transduced cells produced by the method of any one of the embodiments provided herein. In some of any such embodiments, the composition further comprises a cyropreservant.

### BRIEF DESCRIPTION OF THE DRAWINGS

FIG. 1 depicts a graph showing the results from vector titration experiments that demonstrate variation in the maximum transducible frequency of T cells between six donors.

FIGS. 2A and 2B depict results from a study that assessed transduction frequency among T cell subsets from samples obtained from three healthy donors. In each of FIGS. 2A and 2B, the upper left panel shows the T cell composition prior to activation and transduction (input or selected composition or population), the upper right panel shows the resulting transduction frequency in those T cell subsets, and the lower panel shows the proportion of the transduced T cells each population represents.

FIGS. 3A and 3B depict results from a larger scale study that assessed transduction frequency among T cell subsets from samples obtained from six healthy donors. In each of FIGS. 3A and 3B, the upper left panel shows the T cell composition prior to activation and transduction (input or selected composition or population), the upper right panel shows the resulting transduction frequency in those T cell subsets, and the lower panel shows the proportion of the transduced T cells each population represents.

FIG. 4 depicts results from a study that assessed the frequency, in samples obtained from patients with relapsed/refractory large B-cell lymphoma, of selected CD4 and CD8 T cell subpopulations that are CCR7+vs CCR7− by flow cytometry (n=145). The boxes as shown in FIG. 4 indicate the interquartile range, while the whiskers indicate the overall range.

### DETAILED DESCRIPTION

Provided are methods for increasing transduction frequency of primary T cells by selecting for, or otherwise obtaining, a population of primary T cells enriched in surface expression of CCR7, prior to or in connection with carrying out transduction.

In general, retroviral-based vectors, such as lentiviral vectors, can be used to stably integrate genes of interest into cells. Among primary cells, T cells are particularly difficult to transduce with retroviral-based vectors. In some cases, transduction efficiency is improved by first activating the cells with a stimulating agent (e.g. anti-CD3/anti-CD28). Activation has been shown to increase LDL receptor expression in some cases, thereby enhancing the uptake of the lentiviral vectors. Typically, T cells are activated for at least one day (sometimes up to 3 days or more) prior to transduction for use in adoptive T cell therapies. For example, lentiviral transduction protocols for T cells typically require activation at least 24 h prior to transduction (Amirache et al. (2014) Blood, 123:1422-1424). In some instances, available procedures for preparing genetically engineered T cells for adoptive immunotherapy can require the sequential ex vivo steps of selection, activation, transduction and expansion.

In some cases, current methods for transduction are not completely satisfactory, particularly in connection with adoptive cell therapies. For example, as shown herein, transduction frequency can be highly variable among cell populations from different subjects, and that this effect is donor-related. Variability among transduction frequency can likewise result in variability of dosing of therapeutic cell compositions, such as due to highly variable numbers of total cells that may need to be dosed among different subjects to achieve a threshold number of cells that are positive for the heterologous gene (e.g. recombinant receptor, such as a chimeric antigen receptor) or due to variability in the frequency of cells positive for the heterologous gene when dosing strategies are based on a threshold number of total cells. In addition, variability in transduction frequency can also impact processes for preparation and manufacturing of the drug product, such as if transduction frequency is a criteria used to monitor success of an engineering process during one or more steps of the methods, e.g. time to harvest of the cells.

Observations herein demonstrate that there are certain subpopulations of T cells that are more transducible than others. As a result of variability of such subpopulations among different subjects, this may explain the variability in transduction frequency resulting in reduced transduction frequency in some cell populations along with inconsistency in the transduction frequency among a plurality of cell populations from different subjects. In particular, the provided methods are based on the observation that CCR7 expression is highly variable among cell populations from different subjects, and that CCR7+ T cells exhibit a higher transduction frequency than CCR7− T cells. Accordingly, the provided methods are advantageous in that they increase transduction frequency of cell populations while reducing variability in the transduction frequency among cell populations from different patients by including a step whereby primary T cells that are positive for surface expression of CCR7 are selected, or primary T cells enriched in CCR7+ T cells are otherwise obtained, for use in transduction.

The provided methods include transduction of an input population (hereinafter also called input composition) of cells that are enriched and/or selected for cells positive for CCR7. In some embodiments, the provided methods involve selecting primary T cells that are positive for surface expression of CCR7 from a population of primary T cells as described in, e.g., Section I-A, an input population enriched in CCR7+ primary T cells. In some embodiments, the input population enriched in CCR7+ primary T cells is incubated with a viral vector particle containing a heterologous gene (encoding a heterologous or recombinant protein) under conditions to transduce cells in the population. In some cases, the input population enriched in CCR7+ primary T cells is first stimulated under stimulatory conditions, such as by incubation in the presence of a stimulatory reagent capable of activating one or more intracellular signaling domains of one or more components of a TCR complex and/or one or more intracellular signaling domains of one or more costimulatory molecules (e.g. anti-CD3/anti-CD28), and then incubated with the viral vector particle under conditions to transduce cells in the population.

In some embodiments, the provided methods involve selecting primary T cells that are positive for surface expression of CCR7 from a composition of cells that was previously incubated under stimulatory conditions as described herein, e.g., in Section I-B, thereby generating a stimulated population enriched in CCR7+ primary T cells that are subsequently transduced.

Accordingly, provided herein is a method for increasing transduction frequency of primary T cells, the method comprising: (a) selecting primary T cells that are positive for surface expression of CCR7 from a biological sample comprising a population of primary T cells, thereby generating an input population enriched in CCR7+ primary T cells; (b) incubating the input population under stimulatory conditions, thereby generating a stimulated composition, wherein said stimulating conditions comprise the presence of a stimulatory reagent capable of activating one or more intracellular signaling domains of one or more components of a TCR complex and/or one or more intracellular signaling domains of one or more costimulatory molecules; and (c) incubating a viral vector particle comprising a heterologous polynucleotide encoding a recombinant protein with T cells of the stimulated composition, thereby generating a population of transduced cells.

Also provided herein is a method for increasing transduction frequency of primary T cells, the method comprising: (a) incubating an input population of primary T cells enriched in CCR7+ T cells under stimulatory conditions, thereby generating a stimulated composition, wherein said stimulating conditions comprise the presence of a stimulatory reagent capable of activating one or more intracellular signaling domains of one or more components of a TCR complex and/or one or more intracellular signaling domains of one or more costimulatory molecules; and (b) incubating a viral vector particle comprising a heterologous polynucleotide encoding a recombinant protein with T cells of the stimulated composition, thereby generating a population of transduced cells.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Methods for T Cell Transduction

Provided herein is a method of increasing transduction frequency of cells, e.g., primary T cells, that includes selecting cells, e.g., primary T cells, that are positive for surface expression of CCR7 from a composition of cells, and incubating or contacting the selected cells with a retroviral vector particle, e.g. lentiviral vector particles. In some embodiments, the method further includes incubating the primary T cells under stimulatory conditions, as described herein, e.g., in Section I-B. In some embodiments, the primary T cells are incubated under stimulatory conditions after selecting cells that are positive for surface expression of CCR7. In some embodiments, the primary T cells are incubated under stimulatory conditions prior to selecting cells that are positive for surface expression of CCR7. In some aspects, the composition of cells is a composition of primary cells obtained from a subject, e.g., a biological sample, in which, in some cases, a subpopulation or subset of cells has been selected and/or enriched. Features of the composition are provided.

Also provided herein are methods for increasing transduction frequency of primary T cells, the method comprising: (a) selecting primary T cells that are positive for surface expression of CCR7 from a biological sample comprising a population of primary T cells, thereby generating an input population enriched in CCR7+ primary T cells; (b) incubating the input population under stimulatory conditions, thereby generating a stimulated composition; and (c) incubating a viral vector particle comprising a heterologous polynucleotide encoding a recombinant protein with T cells of the stimulated composition, thereby generating a population of transduced cells. In some embodiments, said stimulating conditions comprise the presence of a stimulatory reagent capable of activating one or more intracellular signaling domains of one or more components of a TCR complex and/or one or more intracellular signaling domains of one or more costimulatory molecules.

Also provided herein are methods for increasing transduction frequency of primary T cells, the method comprising incubating a viral vector particle comprising a heterologous polynucleotide encoding a recombinant protein with T cells of an input population of cells enriched in CCR7+ primary T cells, thereby generating a population of transduced cells. In some embodiments, prior to the incubation, cells of the input population of cells have been incubated under stimulatory conditions.

Also provided herein are methods for increasing transduction frequency of primary T cells, the method comprising: (a) incubating an input population of primary T cells enriched in CCR7+ T cells under stimulatory conditions, thereby generating a stimulated composition; and (b) incubating a viral vector particle comprising a heterologous polynucleotide encoding a recombinant protein with T cells of the stimulated composition, thereby generating a population of transduced cells. In some embodiments, said stimulating conditions comprise the presence of a stimulatory reagent capable of activating one or more intracellular signaling domains of one or more components of a TCR complex and/or one or more intracellular signaling domains of one or more costimulatory molecules.

In some of any of the provided embodiments, the incubation under stimulatory conditions results in or leads to activation or stimulation of the cells in the population of cells, and/or are capable of activing or stimulating a signal in the cells of the population of cells, e.g., CD4+ T cells, such as a signal generated from a TCR and/or a coreceptor.

In some embodiments, the cells include one or more nucleic acids (e.g., polynucleotides) introduced via genetic engineering in accord with the provided methods, e.g., as described in Section I-D, and thereby express recombinant or genetically engineered products of such nucleic acids (e.g., polynucleotides). In some embodiments, the nucleic acids (e.g., polynucleotides) are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids (e.g., polynucleotides) are not naturally occurring, such as a nucleic acid not found in nature, including one comprising chimeric combinations of nucleic acids encoding various domains from multiple different cell types.

The processing steps of the methods may include any one or more of a number of cell processing steps, alone or in combination. In particular embodiments, the processing steps include transduction of the cells with viral vector particles containing a retroviral vector, such as one encoding a recombinant product for expression in the cells. The methods may further and/or alternatively include other processing steps, such as steps for the isolation, separation, selection, washing, suspension, dilution, concentration, and/or formulation of the cells. In some cases, the methods also can include an ex vivo step for cultivation (e.g., stimulation of the cells, for example, to induce their proliferation and/or activation). In other cases, a step for stimulating or activating cells is carried out in vivo upon administration of cells to a subject, by recognition of antigen and/or following administration of one or more agents to boost or augment expansion, activation and/or proliferation of cells in the subject. In some embodiments, the methods include isolating cells from the subject, preparing, processing, culturing, and/or engineering them, and re-introducing them into the same subject, before or after cryopreservation.

In some embodiments, the method includes processing steps carried out in an order in which: cells, e.g. primary cells, are first isolated, such as selected or separated, from a biological sample; selected cells are incubated with viral vector particles for transduction; and transduced cells are formulated in a composition. In some cases, transduced cells are activated, expanded or propagated ex vivo, such as by stimulation in the presence of a stimulation reagent, e.g. anti-CD3/anti-CD28 and/or one or more recombinant T cell stimulatory cytokines, such as IL-2, IL-7 and/or IL-25. The activation or stimulation step can, in some embodiments, occur before or after the primary cells undergo one or more selection steps, e.g., selection for cells that are positive for surface expression of CCR7. In some embodiments, the method can include one or more processing steps from among washing, suspending, diluting and/or concentrating cells, which can occur prior to, during or simultaneous with or subsequent to one or more of the isolation, such as separation or selection, transduction, stimulation, and/or formulation steps.

In some embodiments, one or more or all of the processing steps, e.g., isolation, selection and/or enrichment, processing, incubation in connection with transduction and engineering, and formulation steps is carried out using a system, device, or apparatus in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps. In one example, the system is a system as described in International Patent Application, Publication Number WO2009/072003, or US 20110003380 A1. In one example, the system is a system as described in International Publication Number WO2016/073602.

In some embodiments, one or more of the cell processing steps in connection with preparing, processing and/or incubating cells in connection with the provided transduction method can be carried out in the internal cavity of a centrifugal chamber, such as a substantially rigid chamber that is generally cylindrical in shape and rotatable around an axis of rotation, which can provide certain advantages compared to other available methods. In some embodiments, all processing steps are carried out in the same centrifugal chamber. In some embodiments, one or more processing steps are carried out in different centrifugal chambers, such as multiple centrifugal chambers of the same type. Such methods include any of those as described in International Publication Number WO2016/073602.

Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. patent application, Publication No.: US 2008/0171951, and published international patent application, publication no. WO 00/38762, the contents of each of which are incorporated herein by reference in their entirety. Depending on the particular process (e.g. dilution, wash, transduction, formulation), it is within the level of a skilled artisan to choose a particular kit that is appropriate for the process. Exemplary kits for use with such systems include, but are not limited to, single-use kits sold by BioSafe SA under product names CS-430.1, CS-490.1, CS-600.1 or CS-900.2. Exemplary methods for transduction using a centrifugal chamber are described in International patent publication No. WO2016/073602.

In some embodiments, the system is included with and/or placed into association with other instrumentation, including instrumentation to operate, automate, control and/or monitor aspects of the various processing steps performed in the system. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the system, such as a closed system, is sterile. In some embodiments, all connections of components of the system, such as between tubing line and a container via a connector, are made under sterile conditions. In some embodiments, connections are made under laminar flow. In some embodiments, connections are made using a sterile connection device that produces sterile connections, such as sterile welds, between a tubing and a container. In some embodiments, a sterile connection device effects connection under thermal condition high enough to maintain sterility, such as temperatures of at least 200° C., such as at least 260° C. or 300° C.

In some embodiments, the system may be disposable, such as a single-use kit. In some embodiments, a single-use kit can be utilized in a plurality of cycles of a process or processes, such as at least 2, 3, 4, 5 or more times, for example, in processes that occur in a continuous or a semi-continuous manner. In some embodiments, the system, such as a single-use kit, is employed for processing of cells from a single patient.

The centrifugal chamber generally is rotatable around an axis of rotation, and the cavity typically is coaxial with the chamber. In some embodiments, the centrifugal chamber further includes a movable member, such as a piston, which generally is capable of movement (e.g., axial movement) within the chamber, to vary the volume of the cavity. Thus, in particular embodiments, the internal cavity is bound by the side wall and end wall of the chamber and the movable member, and has a variable volume that may be adjusted by moving the movable member. The movable member may be made of rigid, substantially or generally rigid, flexible materials, or combinations thereof.

The chamber generally also includes one or more opening(s), such as one or more inlet, one or more outlet, and/or one or more inlet/outlet, which can permit intake and expression of liquid and/or gas to and from the cavity. In some cases, the opening can be an inlet/outlet where both intake and expression of the liquid and/or gas occurs. In some cases, the one or more inlets can be separate or different from the one or more outlets. The opening or openings may be in one of the end walls. In some embodiments, liquid and/or gas is taken into and/or expressed from the cavity by movement of the movable member to increase and/or decrease the cavity's volume. In other embodiments, liquid and/or gas may be taken into and/or expressed from the cavity through a tubing line or other channel that is or is placed in connection with the opening, for example, by placing the line or channel in connection with and control of a pump, syringe, or other machinery, which may be controlled in an automated fashion.

In some embodiments, the chamber is part of a closed system, such as a sterile system, having various additional components such as tubing lines and connectors and caps, within which processing steps occur. Thus, in some embodiments, the provided methods and/or steps thereof are carried out in a completely closed or semi-closed environment, such as a closed or semi-closed sterile system, facilitating the production of cells for therapeutic administration to subjects without the need for a separate sterile environment, such as a biosafety cabinet or room. The methods in some embodiments are carried out in an automated or partially automated fashion.

In some embodiments, the chamber is associated with a centrifuge, which is capable of effecting rotation of the chamber, such as around its axis of rotation. Rotation may occur before, during, and/or after the incubation in one or more of the processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In aspects of the methods, the processes need not be performed in the same closed system, such as in the same centrifugal chamber, but can be performed under a different closed system, such as in a different centrifugal chamber; in some embodiments, such different centrifugal chambers are at the respective points in the methods placed in association with the same system, such as placed in association with the same centrifuge. In some embodiments, all processing steps are performed in a closed system, in which all or a portion of each one or more processing step is performed in the same or a different centrifugal chamber.

A. Samples and Cell Preparations

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some embodiments, the cells are derived from a biological sample. In some embodiments, the biological sample is a blood sample. In some embodiments, the biological sample is a leukapheresis sample. In some embodiments, the cells are T cells.

The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells are derived from cell lines, e.g., T cell lines. The cells in some embodiments are obtained from a xenogeneic source, for example, from mouse, rat, non-human primate, and pig.

In some embodiments, the cells may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, prior to the enriching and/or selecting of cells, the sample is contacted with and/or contains serum or plasma, such as human serum or plasma. In some embodiments, the serum or plasma is autologous to the subject from which the cells were obtained. In some embodiments, the serum or plasma is present in the sample at a concentration of at least or at least about 10% (v/v), at least or at least about 15% (v/v), at least or at least about 20% (v/v), at least or at least about 25% (v/v), at least or at least about 30% (v/v), at least or at least about 35% (v/v), or at least or at least about 40% (v/v). In some embodiments, prior to the selection and/or transduction of cells, the sample containing primary cells is contacted with or contains an anticoagulant. In some embodiments, the anti-coagulant is or contains free citrate ion, e.g. anticoagulant citrate dextrose solution, Solution A (ACD-A).

In some embodiments, the input composition is free and/or substantially free of serum. In particular embodiments, the input composition is incubated and/or contacted in serum-free media. In some embodiments, the serum free media is a defined and/or well-defined cell culture media. In certain embodiments, the serum free media is a controlled culture media that has been processed, e.g., filtered to remove inhibitors and/or growth factors. In some embodiments, the serum free media contains proteins. In certain embodiments, the serum-free media may contain serum albumin, hydrolysates, growth factors, hormones, carrier proteins, and/or attachment factors. In some embodiments, the serum-free media contains proteins, e.g., albumin, such as bovine serum albumin, human serum albumin, and/or recombinant albumin. In some embodiments, the serum free media contains a basal media, e.g., DMEM or RPMI 1640, containing amino acids, vitamins, inorganic salts, buffers, antioxidants and energy sources. In some embodiments, the serum free media is supplemented, such as with, but not limited to, albumin, chemically defined lipids, growth factors, insulin, cytokines, and/or antioxidants. In some embodiments, the serum free media is formulated to support growth, proliferation, health, homeostasis of cells of a certain cell type, such as immune cells, T cells, and/or CD4+ and CD8+ T cells.

In some embodiments, prior to the selection and/or enrichment of cells, the sample or the cells in the sample can be rested or held prior to further processing steps. In some embodiments, the sample is maintained at or held at a temperature of from or from about 2° C. to 8° C. for up to 48 hours, such as for up to 12 hours, 24 hours or 36 hours.

In some embodiments, the preparation methods include steps for freezing, e.g., cryopreserving, the cells, either before or after isolation, selection and/or enrichment and/or stimulation and/or activation and/or incubation for transduction and engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD4+, CD8+, CD3+, CD28+, and/or CCR7+ T cells, are isolated by positive or negative selection techniques.

For example, CD3+, CD28+ T cells can be positively selected using anti-CD3/anti-CD28 conjugated magnetic beads (e.g., DYNABEADS® M-450 CD3/CD28 T Cell Expander).

In some embodiments, the T cells, e.g., the population of primary T cells, are unfractionated T cells, are enriched or isolated CD3+ T cells, are enriched or isolated CD4+ T cells, or are enriched or isolated CD8+ T cells.

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naïve, memory, and/or effector T cell subpopulations.

In some embodiments, CD8+ cells are further enriched for or depleted of naïve, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In some embodiments, the enrichment for naïve T (T_{N}) or central memory T (T_{CM}) cells is based on positive or high surface expression of one or more of CCR7, CD4, CD8, and CD3. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, or CD8+ cells, or CD3+ cells, or CD4+ and CD8+ cells, where both the negative and positive fractions are retained.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some embodiments, the composition, e.g., input population, comprises at least 80%, at least 85%, at least 90%, or at least 95% cells that are CD4+ T cells or CD8+ T cells. In some embodiments, the composition, e.g., input population, comprises at least 80%, at least 85%, at least 90%, or at least 95% cells that are CD4+ T cells. In some embodiments, the composition, e.g., input population, comprises at least 80%, at least 85%, at least 90%, or at least 95% cells that are CD8+ T cells. In some embodiments, the composition, e.g., input population, comprises at least 80%, at least 85%, at least 90%, or at least 95% cells that are CD4+ T cells and CD8+ T cells. In some embodiments, the ratio of the CD4+ T cells to the CD8+ T cells is or is about 1:1, 1:2, 2:1, 1:3, or 3:1. In some embodiments, the composition, e.g., input population, comprises at least 80%, at least 85%, at least 90%, or at least 95% cells that are CD3+ T cells.

In some embodiments, the method includes the use of a cell population of primary T cells enriched in CCR7+ T cells, such as an input population enriched in CCR7+ primary T cells.

In some embodiments, a population that is enriched in cells positive for surface expression of CCR7 are selected or obtained from a biological sample. In some embodiments, the selecting step comprises selecting primary T cells that are positive for surface expression of CCR7 from a biological sample comprising a population of primary T cells, thereby generating a cell population enriched in CCR7+ primary T cells. In some aspects, the cell population enriched in CCR7+ primary T cells is referred to as an “input population enriched in CCR7+ primary T cells.” In some embodiments, the selection is a positive selection by selecting or isolating cells positive for CCR7 from the biological sample. In some embodiments, the selection is a negative selection by removing or depleting cells negative for CCR7 from the biological sample. The selection can occur before or after the cells are incubated under stimulatory conditions as described herein, e.g., in Section I-B.

In certain embodiments, negative expression, e.g., of a particular protein, such as negative expression of CCR7 or CCR7−, is an expression equal to or less than the level of background expression, e.g., as detected using a standard technique, such as a technique involving antibody-staining with a control antibody not specific to the protein, e.g., isotype antibody. In certain embodiments, negative expression is equal to or less than the level of background expression as detected by suitable techniques for assessing protein or gene expression, such as but not limited to immunohistochemistry, immunofluorescence, or flow cytometry based techniques. In some embodiments, positive expression, e.g., of a particular protein, such as positive expression of CCR7 or CCR7+, is or includes surface expression of the protein in an amount, level, or concentration above background e.g., as detected using a standard technique, such as a technique involving antibody-staining with a control antibody not specific to the protein, e.g. isotype antibody. In certain embodiments, positive expression is greater than the level of background expression as detected by suitable techniques for assessing protein or gene expression, such as but not limited to immunohistochemistry, immunofluorescence, or flow cytometry based techniques. In particular embodiments, the amount, frequency, or percentage of cells that are negative or positive for protein expression, e.g., surface expression, in the sample, composition, or population is determined by flow cytometry.

In certain embodiments, T cells, e.g. CD3+, or subsets of T cells, e.g., CD4+ or CD8+ T cells, are selected, isolated, or enriched from the biological sample prior to selecting, isolating, or enriching CCR7+ T cells from the biological sample. In some embodiments, T cells, e.g. CD3+, or subsets of T cells, e.g., CD4+ or CD8+ T cells, are selected, isolated, or enriched from the population of enriched CCR7+ T cells. In particular embodiments, the selecting, isolating or enriching T cells, e.g. CD3+, or a subset of T cells, e.g. CD4+ or CD8+ T cells, involves positive selection of cells positive for CD3, CD4 or CD8 from the sample.

In particular embodiments, (1) CD4+ T cells are enriched, selected, or isolated from a biological sample, thereby generating a population of enriched CD4+ T cells and a non-selected population enriched for CD4− cells; (2) CD8+ T cells are enriched, selected, or isolated from the non-selected population of enriched CD4− cells, thereby generating a population of enriched CD8+ T cells; and (3) CCR7+ T cells are selected or isolated from the enriched CD4+ and CD8+ T cell populations, generating populations of enriched CCR7+CD4+ and CCR7+CD8+ T cells. In particular embodiments, (1) CD8+ T cells are enriched, selected, or isolated from a biological sample, thereby generating a population of enriched CD8+ T cells and a non-selected population enriched for CD8− cells; (2) CD4+ T cells are enriched, selected, or isolated from the non-selected population of enriched CD4− cells, thereby generating a population of enriched CD4+ T cells; and (3) CCR7+ T cells are selected or isolated from the enriched CD4+ and CD8+ T cell populations, generating populations of enriched CCR7+CD4+ and CCR7+CD8+ T cells.

In particular embodiments, CD4+ T cells are enriched, selected, or isolated from a biological sample, thereby generating an enriched population of CD4+ T cells, and then CCR7+ cells are selected or isolated from the enriched population of CD4+ T cells, thereby generating a population of enriched CCR7+CD4+ T cells. In particular embodiments, CD8+ T cells are enriched, selected, or isolated from a biological sample, thereby generating an enriched population of CD8+ T cells, and then CCR7+ cells are identified or selected from the enriched population of CD8+ T cells, thereby generating a population of enriched CD57-CD8+ T cells.

In particular embodiments, CD3+ T cells are enriched, selected, or isolated from a biological sample, thereby generating an enriched population of CD3+ T cells, and then CCR7+ cells are identified or selected from the enriched population of CD3+ T cells, thereby generating a population of enriched CCR7+CD8+ T cells.

In some embodiments, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the input population enriched in CCR7+ primary T cells are CCR7+ primary T cells. In some embodiments, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the input population enriched in CCR7+ primary T cells are CCR7+ primary T cells. In some embodiments, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the input population enriched in CCR7+ primary T cells are CCR7+ primary T cells. In some embodiments, at least 90%, at least 91%, at least 92%, at least 93%, at least 94%, at least 95%, at least 96%, at least 97%, at least 98%, at least 99%, or 100% of the input population enriched in CCR7+ primary T cells are CCR7+ primary T cells. In some embodiments, at least 95%, at least 96%, at least 97%, at least 98%, at least 99%, or 100% of the input population enriched in CCR7+ primary T cells are CCR7+ primary T cells.

In some embodiments, the selecting step does not comprise selecting for cells that are (a) CCR7+ and CD45RO+; or (b) CCR7+ and CD27+; or (c) CCR7+ and CD45RA−; or (d) CCR7+ and CD62L+; or (e) CCR7+ and CD45RA+; or (f) CCR7+ and CD62L−.

In some embodiments, the input population is not enriched in T cells that are (a) CCR7+ and CD45RO+; or (b) CCR7+ and CD27+; or (c) CCR7+ and CD45RA−; or (d) CCR7+ and CD62L+; or (e) CCR7+ and CD45RA+; or (f) CCR7+ and CD62L−. In some embodiments, less than 50%, less than 55%, less than 60%, less than 65%, less than 70%, less than 75%, less than 80%, less than 85%, or less than 90% of the input population are (a) CCR7+ and CD45RO+; or (b) CCR7+ and CD27+; or (c) CCR7+ and CD45RA−; or (d) CCR7+ and CD62L+; or (e) CCR7+ and CD45RA+; or (f) CCR7+ and CD62L− T cells. In some embodiments, the input population is not enriched in CCR7+ and CD45RO+ T cells, optionally wherein less than 85% of the total cells of the input population are CCR7+ and CD45RO+ T cells. In some embodiments, the input population is not enriched in CCR7+ and CD27+ T cells, optionally wherein less than 85% of the total cells of the input population are CCR7+ and CD27+ T cells. In some embodiments, the input population is not enriched in CCR7+ and CD45RA− T cells, optionally wherein less than 85% of the total cells of the input population are CCR7+ and CD45RA− T cells. In some embodiments, the input population is not enriched in CCR7+ and CD62L+ T cells, optionally wherein less than 85% of the total cells of the input population are CCR7+ and CD62L+ T cells. In some embodiments, the input population is not enriched in CCR7+ and CD45RA+ T cells, optionally wherein less than 85% of the total cells of the input population are CCR7+ and CD45RA+ T cells. In some embodiments, the input population is not enriched in CCR7+ and CD62L− T cells, optionally wherein less than 85% of the total cells of the input population are CCR7+ and CD62L− T cells.

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1. In one example, the system is a system as described in International Publication Number WO2016/073602.

In some embodiments, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber.

For example, immunoaffinity-based selection can depend upon a favorable energetic interaction between the cells being separated and the molecule specifically binding to the marker on the cell, e.g., the antibody or other binding partner on the solid surface, e.g., particle. In certain available methods for affinity-based separation using particles such as beads, particles and cells are incubated in a container, such as a tube or bag, while shaking or mixing, with a constant cell density-to-particle (e.g., bead) ratio to aid in promoting energetically favored interactions. Such approaches may not be ideal for use with large-scale production, for example, in that they may require use of large volumes in order to maintain an optimal or desired cell-to-particle ratio while maintaining the desired number of cells. Accordingly, such approaches can require processing in batch mode or format, which can require increased time, number of steps, and handling, increasing cost and risk of user error.

In some embodiments, by conducting such selection steps or portions thereof (e.g., incubation with antibody-coated particles, e.g., magnetic beads) in the cavity of the centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g., bead reagent) used in the selection, and thus the chemical potential of the solution, without affecting the total number of cells in the cavity. This, in turn, can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70%, or no more than 80% of the amount of the same selection reagent(s) employed for selection of cells in a tube or container-based incubation for the same number of cells and/or the same volume of cells according to manufacturer's instructions is employed.

The incubation with a selection reagent or reagents, e.g., as part of selection methods which may be performed in the chamber cavity, include using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g., is no more than 5%, 10%, 20%, 30%, 40%, 50%, 60%, 70%, or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or about at least or about or 10 mL, 20 mL, 30 mL, 40 mL, 50 mL, 60 mL, 70 mL, 80 mL, 90 mL, 100 mL, 150 mL, or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from or from about 5 minutes to 6 hours, such as 30 minutes to 3 hours, for example, at least or about at least 30 minutes, 60 minutes, 120 minutes, or 180 minutes.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g., at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the further selection is performed outside of the centrifugal chamber. In some embodiments, the separation is performed in the same closed system in which the centrifugal chamber is present and in which the incubation of cells with the selection reagent was performed. In some embodiments, after incubation with the selection reagents, incubated cells, including cells in which the selection reagent has bound, are expressed from the centrifugal chamber, such as transferred out of the centrifugal chamber, into a system for immunoaffinity-based separation of the cells. In some embodiments, the system for immunoaffinity-based separation is or contains a magnetic separation column. In some embodiments, prior to separation, one or more other processing steps can be performed in the chamber, such as washing.

In some aspects, the separation and/or other steps is carried out using CliniMACS system (Miltenyi Biotic), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the provided retroviral particles can transduce stimulated and/or activated T cells. In particular embodiments, the provided retroviral particles can transduce resting T cells. In some embodiments, the input composition comprises a plurality of cells, such as immune cells, e.g. T cells, that are non-cycling and/or quiescent and/or resting and/or in which a majority of cells, e.g. greater than 50%, 60%, 70%, 80%, 80% or more cells, in a population so transduced are non-cycling and/or quiescent and/or resting. In some embodiments, the input composition comprises a population of T cells in which at least 40%, 50%, 60%, 70%, 80%, 90% or more of the T cells in the population are resting T cells, such as T cells that lack a T cell activation marker, such as a surface marker or intracellular cytokine or other marker, and/or T cells that are in the G₀ or G₀G₁ₐ stage of the cell cycle. In some embodiments, the cells are in the G₀, G₀/G₁ₐ or G1 stage of the cell cycle.

In some embodiments, the cells that are transduced were, prior to transduction, incubated under stimulatory conditions. In some embodiments, at least 10%, at least 20%, at least 30%, at least 40%, at least 50%, or at least 60% of the cells that are transduced (i) express a surface marker selected from the group consisting of HLA-DR, CD25, CD69, CD71, CD40L and 4-1BB; (ii) comprise intracellular expression of a cytokine selected from the group consisting of IL-2, IFN-gamma, TNF-alpha; (iii) are in the G1 or later phase of the cell cycle; and/or (iv) are capable of proliferating.

B. Activation and Stimulation

In some embodiments, the provided methods are used in connection with incubating cells under stimulating conditions. In some embodiments, the stimulating conditions include conditions that activate or stimulate, and/or are capable of activing or stimulating a signal in the cell, e.g., a CD4+ T cell, such as a signal generated from a TCR and/or a coreceptor. In some embodiments, the stimulating conditions include one or more steps of culturing, cultivating, incubating, activating, propagating the cells with and/or in the presence of a stimulatory reagent, e.g., a reagent that activates or stimulates, and/or is capable of activing or stimulating a signal in the cell. In some embodiments, the stimulatory reagent stimulates and/or activates a TCR and/or a coreceptor. In particular embodiments, the stimulatory reagent is a reagent provided herein, e.g., as described in Section I-B-1.

In certain embodiments, one or more compositions of enriched T cells are incubated under stimulating conditions prior to genetically engineering the cells, e.g., transfecting and/or transducing the cells, such as by a method or technique provided herein, e.g., a method or technique described in Section I-C and I-D. In particular embodiments, the composition of enriched T cells that is incubated under stimulating conditions is an input composition. In certain embodiments, the cells of the input compositions have previously been isolated, selected, enriched, or obtained from a biological sample. In particular embodiments, the cells from the input composition have been previously cryofrozen and stored, and are thawed prior to the incubation.

In some embodiments, the provided methods are used in connection with the one or more processing steps that include a step of stimulating cells, such as cells from the input compositions. In certain embodiments, the incubation may be prior to or in connection with genetic engineering, such as genetic engineering resulting from embodiments of transduction described herein, e.g., methods described in Section I-D. In some embodiments, the stimulation results in activation and/or proliferation of the cells, for example, prior to engineering, e.g., transduction.

In some embodiments, the processing steps include incubations of cells, such as input cells and/or cells of the input composition, in which the incubation steps can include culture, cultivation, stimulation, activation, and/or propagation of cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

In certain embodiments, the cells, e.g., cells of the input composition, are incubated e.g., under stimulating conditions such as in the presence of a stimulatory reagent, at a density of less than at or about 5×10⁷ cells/mL, 4×10⁷ cells/mL, 3×10⁷ cells/mL, 2×10⁷ cells/mL, 1×10⁷ cells/mL, 9×10⁶ cells/mL, 8×10⁶ cells/mL, 7×10⁶ cells/mL, 6×10⁶ cells/mL, 5×10⁶ cells/mL, 4×10⁶ cells/mL, or 3×10⁶ cells/mL. In particular embodiments, the cells are incubated at a density of less than 5×10⁶ cells/mL. In some embodiments, the cells are incubated at a density of between 1×10³ cells/mL and 1×10⁹ cells/mL, 1×10⁴ cells/mL and 1×10⁸ cells/mL, 1×10⁵ cells/mL and 1×10⁷ cells/mL, 5×10⁵ cells/mL and 1×10⁷ cells/mL, 1×10⁶ cells/mL and 5×10⁶ cells/mL, or 3×10⁶ cells/mL and 5×10⁶ cells/mL. In particular embodiments, the cells are incubated at a density of or of about 1×10⁶ cells/mL, 1.5×10⁶ cells/mL, 2×10⁶ cells/mL, 2.5×10⁶ cells/mL, 3×10⁶ cells/mL, 3.5×10⁶ cells/mL, 4×10⁶ cells/mL, 4.5×10⁶ cells/mL, or 5×10⁶ cells/mL. In particular embodiments, the cells are incubated at a density of or of about 3×10⁶ cells/mL. In some embodiments, the cells are viable cells. In certain embodiments, the cells are negative for an apoptotic marker, e.g., Annexin V or active caspase 3. In particular embodiments, the cells are or include CD4+ T cells and CD8+ T cells.

In particular embodiments, indicators of viability include but are not limited to, indicators of cellular replication, mitochondrial function, energy balance, membrane integrity and cell mortality. In certain embodiments, the indicators of viability further include indicators of oxidative stress, metabolic activation, metabolic stability, enzyme induction, enzyme inhibition, and interaction with cell membrane transporters. In some embodiments, the viable cells include cells undergoing normal functional cellular processes and/or cell that have not undergone or are not under the process of undergoing necrosis or programmed cell death. In some embodiments, viability can be assessed by the redox potential of the cell, the integrity of the cell membrane, or the activity or function of mitochondria. In some embodiments, viability is the absence of a specific molecule associated with cell death, or the absence of the indication of cell death in an assay. In certain embodiments, the viability of cells can be detected, measured, and/or assessed by a number of routine means. Non-limiting examples of such viability assays include, but are not limited to, dye uptake assays (e.g., calcein AM assays), XTT cell viability assays, and dye exclusion assays (e.g., trypan blue, Eosin, or propidium dye exclusion assays). Viability assays are useful for determining the number or percentage (e.g., frequency) of viable cells in a cell dose, a cell composition, and/or a cell sample.

In particular embodiments, the apoptotic marker may include any known marker associated with apoptosis, and may include expression of genes, proteins, or active forms of proteins, or the appearance of features associated with apoptosis, such as blebbing and/or nuclear breakdown. In certain embodiments, the apoptotic marker is a marker associated with apoptosis that may include, but is not limited to, pro-apoptotic factors known to initiate apoptosis, members of the death receptor pathway, activated members of the mitochondrial (intrinsic) pathway, Bcl-2 family members such as Bax, Bad, and Bid, Fas, FADD, presence of nuclear shrinkage (e.g., monitored by microscope), presence of chromosomal DNA fragmentation (e.g., presence of chromosomal DNA ladder), or markers associated with apoptosis assays, e.g., TUNEL staining, and Annexin V staining. In some embodiments, the marker of apoptosis is caspase expression, e.g., expression of the active forms of caspase-1, caspase-2, caspase-3, caspase-7, caspase-8, caspase-9, caspase-10 and/or caspase-13. In some embodiments, the apoptotic marker is Annexin V. In certain embodiments, the apoptotic marker is active caspase-3.

In some embodiments, between at or about 1×10⁵ and at or about 500,000×10⁶ cells, between at or about 1×10⁶ and at or about 50,000×10⁶ cells, between at or about 10×10⁶ and at or about 5,000×10⁶ cells, between at or about 1×10⁶ and at or about 1,000×10⁶ cells, between at or about 50×10⁶ and at or about 5,000×10⁶ cells, between at or about 10×10⁶ and at or about 1,000×10⁶ cells, between at or about 100×10⁶ and at or about 2,500×10⁶ cells, between at or about 100×10⁶ and at or about 500×10⁶ cells, between at or about 200×10⁶ and at or about 400×10⁶ cells, e.g., cells of the input composition, are incubated e.g., under stimulating conditions such as in the presence of a stimulatory reagent. In particular embodiments, at least, at, or at about 50×10⁶ cells, 100×10⁶ cells, 150×10⁶ cells, 200×10⁶ cells, 250×10⁶ cells, 300×10⁶ cells, 350×10⁶ cells, 400×10⁶ cells, 450×10⁶ cells, or 500×10⁶ cells are incubated, e.g., under stimulating conditions. In some embodiments, the cells are viable cells. In certain embodiments, the cells are negative for a marker of apoptosis, e.g., Annexin V or active caspase 3. In particular embodiments, the cells are or include CD4+ T cells and CD8+ T cells.

In some embodiments, between at or about 1×10⁵ and at or about 25,000×10⁶, between at or about 1×10⁶ and at or about 25,000×10⁶, between at or about 10×10⁶ and at or about 2,500×10⁶, between at or about 1×10⁶ and at or about 500×10⁶, between at or about 50×10⁶ and at or about 2,500×10⁶, between at or about 10×10⁶ and at or about 500×10⁶, between at or about 100×10⁶ and at or about 500×10⁶, between at or about 200×10⁶ and at or about 400×10⁶, between at or about 50×10⁶ and at or about 300×10⁶ CD4+ T cells, e.g., CD4+ T cells of the input composition, are incubated e.g., under stimulating conditions such as in the presence of a stimulatory reagent. In particular embodiments, at least, at, or at about 25×10⁶, 50×10⁶, 75×10⁶, 100×10⁶, 125×10⁶, 150×10⁶, 175×10⁶, 200×10⁶, 225×10⁶, or 250×10⁶ CD4+ T cells are incubated, e.g., under stimulating conditions. In some embodiments, the CD4+ T cells are viable CD4+ T cells. In certain embodiments, the CD4+ T cells are negative for a marker of apoptosis, e.g., Annexin V or active caspase 3.

In certain embodiments, between at or about 1×10⁵ and at or about 25,000×10⁶, between at or about 1×10⁶ and at or about 25,000×10⁶, between at or about 10×10⁶ and at or about 2,500×10⁶, between at or about 1×10⁶ and at or about 500×10⁶, between at or about 50×10⁶ and at or about 2,500×10⁶, between at or about 10×10⁶ and at or about 500×10⁶, between at or about 100×10⁶ and at or about 500×10⁶, between at or about 200×10⁶ and at or about 400×10⁶, between at or about 50×10⁶ and at or about 300×10⁶ CD8+ T cells, e.g., CD8+ T cells of the input composition, are incubated e.g., under stimulating conditions such as in the presence of a stimulatory reagent. In some embodiments, at least, at, or at about 25×10⁶, 50×10⁶, 75×10⁶, 100×10⁶, 125×10⁶, 150×10⁶, 175×10⁶, 200×10⁶, 225×10⁶, or 250×10⁶ CD8+ T cells are incubated, e.g., under stimulating conditions. In some embodiments, the CD8+ T cells are viable CD8+ T cells. In certain embodiments, the CD8+ T cells are negative for a marker of apoptosis, e.g., Annexin V or active caspase 3.

In some embodiments, the conditions for stimulation and/or activation can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulatory reagents include a primary agent, which can be any stimulatory reagent described herein. In some embodiments, the stimulatory reagents include a primary agent and a secondary agent. The secondary agent can, in some embodiments, be any stimulatory reagent described herein. In some embodiments, the primary agent specifically binds to a member of a TCR complex, optionally that specifically binds to CD3. In some embodiments, the secondary agent specifically binds to a T cell costimulatory molecule, optionally wherein the costimulatory molecule is selected from CD28, CD137 (4-1-BB), OX40, or ICOS.

In some embodiments, the stimulating conditions or stimulatory reagents include one or more reagent, e.g., ligand, which is capable of binding (e.g., specifically binding) to a member of a TCR complex. In some embodiments, the member of a TCR complex is CD3. In some embodiments, the stimulating conditions or stimulatory reagents include one or more reagent, e.g., ligand, which is capable of stimulating or activating an intracellular signaling domain of a TCR complex. In some aspects, the agent turns on or initiates TCR/CD3 intracellular signaling cascade in a T cell, such as agents suitable to deliver a primary signal, e.g., to initiate activation of an ITAM-induced signal, such as those specific for a TCR component, e.g., anti-CD3, and/or an agent that promotes a costimulatory signal, such as one specific for a T cell costimulatory receptor, e.g., anti-CD28, or anti-4-1BB, for example, bound to solid support such as a bead, and/or one or more cytokines. In some embodiments, the agent that specifically binds to a T cell costimulatory molecule is an agent that specifically binds to CD28, CD137 (4-1BB), OX40, or ICOS. Among the stimulatory reagents are anti-CD3/anti-CD28 beads (e.g., DYNABEADS® M-450 CD3/CD28 T Cell Expander, and/or ExpACT® beads). Optionally, the expansion method may further comprise the step of adding anti-CD3 and/or anti CD28 antibody to the culture medium. In some embodiments, the stimulating agents include cytokines.

In particular embodiments, the stimulating conditions include incubating, culturing, and/or cultivating the cells with a stimulatory reagent. In particular embodiments, the stimulatory reagent is a reagent provided herein, e.g., a reagent described in Section I-B-1. In certain embodiments, the stimulatory reagent contains or includes a bead. In certain embodiments, the start and or initiation of the incubation, culturing, and/or cultivating cells under stimulating conditions occurs when the cells are come into contact with and/or are incubated with the stimulatory reagent. In particular embodiments, the cells are incubated prior to, during, and/or subsequent to genetically engineering the cells, e.g., introducing a recombinant polynucleotide into the cell such as by transduction or transfection.

In some embodiments, the composition of enriched T cells is incubated at a ratio of stimulatory reagent and/or beads to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of stimulatory reagent and/or beads to cells is between 2.5:1 and 0.2:1, between 2:1 and 0.5:1, between 1.5:1 and 0.75:1, between 1.25:1 and 0.8:1, or between 1.1:1 and 0.9:1. In particular embodiments, the ratio of stimulatory reagent to cells is about 1:1 or is 1:1.

In particular embodiments, the stimulating conditions include incubating, culturing, and/or cultivating the cells, e.g., cells from an input composition, with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes IL-2.

In certain embodiments, the amount or concentration of the one or more cytokines are measured and/or quantified with International Units (IU). International units may be used to quantify vitamins, hormones, cytokines, vaccines, blood products, and similar biologically active substances. In some embodiments, IU are or include units of measure of the potency of biological preparations by comparison to an international reference standard of a specific weight and strength e.g., WHO 1st International Standard for Human IL-2, 86/504. International Units are the only recognized and standardized method to report biological activity units that are published and are derived from an international collaborative research effort. In particular embodiments, the IU for composition, sample, or source of a cytokine may be obtained through product comparison testing with an analogous WHO standard product. For example, in some embodiments, the IU/mg of a composition, sample, or source of human recombinant IL-2, IL-7, or IL-15 is compared to the WHO standard IL-2 product (NIBSC code: 86/500), the WHO standard IL-17 product (NIBSC code: 90/530) and the WHO standard IL-15 product (NIBSC code: 95/554), respectively.

In some embodiments, the biological activity in IU/mg is equivalent to (ED₅₀ in ng/mL)⁻¹×10⁶. In particular embodiments, the ED₅₀ of recombinant human IL-2 or IL-15 is equivalent to the concentration required for the half-maximal stimulation of cell proliferation (XTT cleavage) with CTLL-2 cells. In certain embodiments, the ED₅₀ of recombinant human IL-7 is equivalent to the concentration required for the half-maximal stimulation for proliferation of PHA-activated human peripheral blood lymphocytes. Details relating to assays and calculations of IU for IL-2 are discussed in Wadhwa et al., Journal of Immunological Methods (2013), 379 (1-2): 1-7; and Gearing and Thorpe, Journal of Immunological Methods (1988), 114 (1-2): 3-9; details relating to assays and calculations of IU for IL-15 are discussed in Soman et al. Journal of Immunological Methods (2009) 348 (1-2): 83-94.

In some embodiments, the cells, e.g., the input cells, are incubated with a cytokine, e.g., a recombinant human cytokine, at a concentration of between at or about 1 IU/mL and at or about 1,000 IU/mL, between at or about 10 IU/mL and at or about 50 IU/mL, between at or about 50 IU/mL and at or about 100 IU/mL, between at or about 100 IU/mL and at or about 200 IU/mL, between at or about 100 IU/mL and at or about 500 IU/mL, between at or about 250 IU/mL and at or about 500 IU/mL, or between at or about 500 IU/mL and at or about 1,000 IU/mL.

In some embodiments, the cells, e.g., the input cells, are incubated with IL-2, e.g., human recombinant IL-2, at a concentration between at or about 1 IU/mL and at or about 500 IU/mL, between at or about 10 IU/mL and at or about 250 IU/mL, between at or about 50 IU/mL and at or about 200 IU/mL, between at or about 50 IU/mL and at or about 150 IU/mL, between at or about 75 IU/mL and at or about 125 IU/mL, between at or about 100 IU/mL and at or about 200 IU/mL, or between at or about 10 IU/mL and at or about 100 IU/mL, e.g., in a serum-free medium. In particular embodiments, cells, e.g., cells of the input composition, are incubated with recombinant IL-2 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 100 IU/mL. In some embodiments, the cells, e.g., the input cells, are incubated in the presence of or of about 100 IU/mL of recombinant IL-2, e.g., human recombinant IL-2.

In some embodiments, the cells, e.g., the input cells, are incubated with recombinant IL-7, e.g., human recombinant IL-7, at a concentration between at or about 100 IU/mL and at or about 2,000 IU/mL, between at or about 500 IU/mL and at or about 1,000 IU/mL, between at or about 100 IU/mL and at or about 500 IU/mL, between at or about 500 IU/mL and at or about 750 IU/mL, between at or about 750 IU/mL and at or about 1,000 IU/mL, or between at or about 550 IU/mL and at or about 650 IU/mL, e.g., in a serum-free medium. In particular embodiments, the cells, e.g., the input cells, are incubated with IL-7 at a concentration at or at about 50 IU/mL, 100 IU/mL, 150 IU/mL, 200 IU/mL, 250 IU/mL, 300 IU/mL, 350 IU/mL, 400 IU/mL, 450 IU/mL, 500 IU/mL, 550 IU/mL, 600 IU/mL, 650 IU/mL, 700 IU/mL, 750 IU/mL, 800 IU/mL, 750 IU/mL, 750 IU/mL, 750 IU/mL, or 1,000 IU/mL. In particular embodiments, the cells, e.g., the input cells, are incubated in the presence of or of about 600 IU/mL of IL-7, e.g., human recombinant IL-7.

In some embodiments, the cells, e.g., the input cells, are incubated with recombinant IL-15, e.g., human recombinant IL-15, at a concentration between at or about 1 IU/mL and at or about 500 IU/mL, between at or about 10 IU/mL and at or about 250 IU/mL, between at or about 50 IU/mL and at or about 200 IU/mL, between at or about 50 IU/mL and at or about 150 IU/mL, between at or about 75 IU/mL and at or about 125 IU/mL, between at or about 100 IU/mL and at or about 200 IU/mL, or between at or about 10 IU/mL and at or about 100 IU/mL, e.g., in a serum-free medium. In particular embodiments, cells, e.g., a cell of the input composition, are incubated with recombinant IL-15 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 200 IU/mL. In some embodiments, the cells, e.g., the input cells, are incubated in the presence of or of about 100 IU/mL of recombinant IL-15, e.g., human recombinant IL-15.

In particular embodiments, the cells, e.g., cells from the input composition, are incubated under stimulating conditions in the presence of IL-2, IL-7, and/or IL-15, e.g., in a serum-free medium. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15. In certain embodiments, the cells are incubated under stimulating conditions in the presence of recombinant IL-2, IL-7, and IL-15, e.g., in a serum-free medium.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the incubation is performed in serum free media. In some embodiments, the serum free media is a defined and/or well-defined cell culture media. In certain embodiments, the serum free media is a controlled culture media that has been processed, e.g., filtered to remove inhibitors and/or growth factors. In some embodiments, the serum free media contains proteins. In certain embodiments, the serum-free media may contain serum albumin, hydrolysates, growth factors, hormones, carrier proteins, and/or attachment factors.

In some embodiments, at least a portion of the incubation in the presence of one or more stimulating conditions or a stimulatory reagent is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g., is no more than 5%, 10%, 20%, 30%, 40%, 50%, 60%, 70%, or 80% of the amount) as compared to the amount of the stimulating agent that is typically used or would be necessary to achieve about the same or similar efficiency of selection of the same number of cells or the same volume of cells when selection is performed without mixing in a centrifugal chamber, e.g., in a tube or bag with periodic shaking or rotation. In some embodiments, the incubation is performed with the addition of an incubation buffer to the cells and stimulating agent to achieve a target volume with incubation of the reagent of, for example, 10 mL to 200 mL, such as at least or at least about or about or 10 mL, 20 mL, 30 mL, 40 mL, 50 mL, 60 mL, 70 mL, 80 mL, 90 mL, 100 mL, 150 mL, or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which can aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from or from about 600 rpm to or to about 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation under stimulating conditions, e.g., with the stimulatory reagent, is between or between about 1 hour and 96 hours, 1 hour and 72 hours, 1 hour and 48 hours, 4 hours and 36 hours, 8 hours and 30 hours, 12 hours and 24 hours, 18 hours and 30 hours, such as at least or at least about 6 hours, 12 hours, 18 hours, 24 hours, 36 hours, or 72 hours. In some embodiments, the total duration of the incubation, e.g., with the stimulatory reagent, is between or between about 18 hours and about 30 hours.

In some embodiments, the cells are cultured, cultivated, and/or incubated under stimulating conditions prior to and/or during a step for introducing a polynucleotide, e.g., a polynucleotide encoding a recombinant receptor, to the cells, e.g., by transduction and/or transfection, such as described by Section I-C. In certain embodiments the cells are cultured, cultivated, and/or incubated under stimulating conditions for an amount of time between 30 minutes and 2 hours, between 1 hour and 8 hours, between 6 hours and 12 hours, between 12 hours and 18 hours, between 16 hours and 24 hours, between 18 hours and 30 hours, between 24 hours and 48 hours, between 24 hours and 72 hours, between 42 hours and 54 hours, between 60 hours and 120 hours between 96 hours and 120 hours, between 90 hours and between 1 days and 7 days, between 3 days and 8 days, between 1 day and 3 days, between 4 days and 6 days, or between 4 days and 5 days prior to the genetic engineering. In some embodiments, the cells are incubated under stimulating conditions for or for about between 18 hours and 30 hours. In particular embodiments, the cells are incubated under stimulating conditions for or for about 24 hours.

In some embodiments, incubating the cells under stimulating conditions includes incubating the cells with a stimulatory reagent that is described in Section I-B-1. In some embodiments, the stimulatory reagent contains or includes a bead, such as a paramagnetic bead, and the cells are incubated with the stimulatory reagent at a ratio of less than 3:1 (beads:cells), such as a ratio of 1:1. In particular embodiments, the cells are incubated with the stimulatory reagent in the presence of one or more cytokines. In some embodiments, the cells are incubated with the stimulatory reagent at a ratio of 1:1 (beads:cells) in the presence of recombinant IL-2, IL-7, and IL-15.

In particular embodiments, an input composition of cells containing CD4+ and CD8+ T cells are incubated under stimulating conditions. In certain embodiments, the cells are incubated in serum free media. In particular embodiments, the input composition contains a ratio of CD4+ T cells to CD8+ T cells of or of about 1:1. In some embodiments, the input composition contains a ratio of CD4+ T cells to CD8+ T cells of or of about 1:1, 1:2, 2:1, 1:3, or 3:1. In certain embodiments at least at or about 100×10⁶ cells, e.g., cells from the input composition, are incubated, such as at a density of less than at or about 5×10⁶ cells/mL, under stimulating conditions. In particular embodiments, at least at or about 50×10⁶ CD4+ T cells and at least at or about 50×10⁶ CD8+ T cells are incubated under stimulating conditions. In some embodiments, the cells are incubated for between 18 hours and 30 hours. In particular embodiments, incubating the cells under stimulating conditions includes incubating the cells with a stimulatory reagent in the presence of IL-2, IL-7, and/or IL-15. In certain embodiments, the cells are incubated with the stimulatory reagent at a ratio of less than 3:1 stimulatory reagent to cells. In some embodiments, the cells are incubated with between at or about 50 IU/mL and at or about 200 IU/mL IL-2, between at or about 400 and at or about 1,000 IU/mL IL-7, and/or between at or about 50 IU/mL and at or about 200 IU/mL IL-15.

In certain embodiments, the between 100×10⁶ and 500×10⁶ cells of an input composition containing CD4+ and CD8+ T at a ratio of or of about 1:1, are incubated under stimulating conditions. In certain embodiments, the between 200×10⁶ and 400×10⁶ cells of an input composition containing CD4+ and CD8+ T at a ratio of or of about 1:1, are incubated under stimulating conditions. In certain embodiments, the cells are viable cells and/or are negative for an apoptotic marker. In some embodiments, at or about 300×10⁶ cells of the input composition are incubated. In particular embodiments, the cells are incubated in serum free media. In particular embodiments, the cells are incubated at a density of or of about 3×10⁶ cells/mL. In some embodiments, at or about 150×10⁶ CD4+ T cells and at or about 150×10⁶ CD8+ T cells are incubated. In particular embodiments, the cells are incubated with a stimulatory reagent at a ratio of or of about 1:1 stimulatory reagent to cells. In certain embodiments, the cells are incubated in the presence of or of about 100 IU/mL IL-2, of or of about 600 IU/mL IL-7, and between 50 IU/mL and/or of or of about 200 IU/mL IL-15.

1. Stimulatory Reagents

In some embodiments, incubating a composition of enriched cells under stimulating conditions is or includes incubating and/or contacting the composition of enriched cells with a stimulatory reagent that is capable of activating and/or expanding T cells. In some embodiments, the stimulatory reagent is capable of stimulating and/or activating one or more signals in the cells. In some embodiments, the one or more signals are mediated by a receptor. In particular embodiments, the one or more signals are or are associated with a change in signal transduction and/or a level or amount of secondary messengers, e.g., cAMP and/or intracellular calcium, a change in the amount, cellular localization, confirmation, phosphorylation, ubiquitination, and/or truncation of one or more cellular proteins, and/or a change in a cellular activity, e.g., transcription, translation, protein degradation, cellular morphology, activation state, and/or cell division. In particular embodiments, the stimulating conditions include incubating, culturing, and/or cultivating the cells with a stimulatory reagent. In certain embodiments, the stimulatory reagent contains or includes a bead. In certain embodiments, the initiation of the stimulation occurs when the cells are incubated or contacted with the stimulatory reagent. In particular embodiments, the stimulatory reagent contains or includes an oligomeric reagent, e.g., a streptavidin mutein oligomer. In particular embodiments, the stimulatory reagent activates and/or is capable of activating one or more intracellular signaling domains of one or more components of a TCR complex and/or one or more intracellular signaling domains of one or more costimulatory molecules. In some embodiments, any of the stimulatory reagents is also referred to as a primary agent, and/or any of the stimulatory reagents is also referred to as a secondary agent. In some embodiments, the stimulatory reagent comprises a primary agent, e.g., a primary agent that specifically binds to a member of a TCR complex. In some embodiments, the primary agent specifically binds to CD3. In some embodiments, the stimulatory reagent comprises a secondary agent, e.g., a secondary agent that specifically binds to a T cell costimulatory molecule. In some embodiments, the secondary agent specifically binds to CD28, CD137 (4-1-BB), OX40, or ICOS.

In some embodiments, the stimulating conditions or stimulatory reagents include one or more agent, e.g., ligand, which is capable of activating an intracellular signaling domain of a TCR complex. In some embodiments, an agent as contemplated herein can include, but is not limited to, RNA, DNA, proteins (e.g., enzymes), antigens, polyclonal antibodies, monoclonal antibodies, antibody fragments, carbohydrates, lipids lectins, or any other biomolecule with an affinity for a desired target. In some embodiments, the desired target is a T cell receptor and/or a component of a T cell receptor. In certain embodiments, the desired target is CD3. In certain embodiment, the desired target is a T cell costimulatory molecule, e.g., CD28, CD137 (4-1-BB), OX40, or ICOS. The one or more agents may be attached directly or indirectly to the bead by a variety of methods known and available in the art. The attachment may be covalent, noncovalent, electrostatic, or hydrophobic and may be accomplished by a variety of attachment means, including for example, a chemical means, a mechanical means, or an enzymatic means. In some embodiments, the agent is an antibody or antigen binding fragment thereof, such as a Fab. In some embodiments, a biomolecule (e.g., a biotinylated anti-CD3 antibody) may be attached indirectly to the bead via another biomolecule (e.g., anti-biotin antibody) that is directly attached to the bead.

In some embodiments, the stimulatory reagent contains one or more agent (e.g., antibody or antigen binding fragment thereof, such as a Fab) that specifically binds to one or more of the following macromolecules on a cell (e.g., a T cell): CD2, CD3, CD4, CD5, CD8, CD25, CD27, CD28, CD29, CD31, CD44, CD45RA, CD45RO, CD54 (ICAM-1), CD127, MHCI, MHCII, CTLA-4, ICOS, PD-1, OX40, CD27L (CD70), 4-1BB (CD137), 4-1BBL, CD30L, LIGHT, IL-2R, IL-12R, IL-1R, IL-15R; IFN-gammaR, TNF-alphaR, IL-4R, IL-10R, CD18/CD11a (LFA-1), CD62L (L-selectin), CD29/CD49d (VLA-4), Notch ligand (e.g. Delta-like 1/4, Jagged 1/2, etc.), CCR1, CCR2, CCR3, CCR4, CCR5, CCR7, and CXCR3 or fragment thereof including the corresponding ligands to these macromolecules or fragments thereof. In some embodiments, the stimulatory reagent contains one or more agent (e.g., antibody or antigen binding fragment thereof, such as a Fab) that specifically binds to one or more of the following macromolecules on a cell (e.g., a T cell): CD28, CD62L, CCR7, CD27, CD127, CD3, CD4, CD8, CD45RA, and/or CD45RO. In some embodiments, the one or more agent is or is capable of being attached to a bead (e.g., a paramagnetic bead). In some embodiments, the one or more agent is or is capable of being attached (e.g., reversibly attached) to an oligomeric reagent, e.g., a streptavidin mutein oligomer.

In some embodiments, the one or more agent comprises an antibody or antigen binding fragment thereof, such as a Fab. The antibody can include a polyclonal antibody, monoclonal antibody (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). In some embodiments, the stimulatory reagent is or comprises an antibody fragment (including antigen-binding fragment), e.g., a Fab, Fab′-SH, Fv, scFv, or (Fab′)2 fragment. It will be appreciated that constant regions of any isotype can be used for the antibodies contemplated herein, including IgG, IgM, IgA, IgD, and IgE constant regions, and that such constant regions can be obtained from any human or animal species (e.g., murine species). In some embodiments, the agent is or comprises an antibody that binds to and/or recognizes one or more components of a T cell receptor. In particular embodiments, the agent is or comprises an anti-CD3 antibody. In certain embodiments, the agent is or comprises an antibody that binds to and/or recognizes a co-receptor. In some embodiments, the stimulatory reagent is or comprises an anti-CD28 antibody. In some embodiments, the stimulatory reagent comprises a primary agent that is or comprises an anti-CD3 antibody, or an antigen-binding fragment thereof, and comprises a secondary agent that is or comprises an an anti-CD28 antibody, or an antigen-binding fragment thereof.

In some embodiments, the cells, e.g., cells of the input population, are stimulated in the presence of a ratio of stimulatory reagent to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of stimulatory reagent to cells is between 2.5:1 and 0.2:1, between 2:1 and 0.5:1, between 1.5:1 and 0.75:1, between 1.25:1 and 0.8:1, or between 1.1:1 and 0.9:1. In particular embodiments, the ratio of stimulatory reagent to cells is about 1:1 or is 1:1.

In some embodiments, the cells are stimulated in the presence of, of about, or of at least 0.01 μg, 0.02 μg, 0.03 μg, 0.04 μg, 0.05 μg, 0.1 μg, 0.2 μg, 0.3 μg, 0.4 μg, 0.5 μg, 0.75 μg, 1 μg, 2 μg, 3 μg, 4 μg, 5 μg, 6 μg, 7 μg, 8 μg, 9 μg, or 10 μg of the stimulatory reagent per 10⁶ cells. In some embodiments, the cells are stimulated in the presence of or of about 4 μg per 10⁶ cells. In particular embodiments, the cells are stimulated in the presence of or of about 0.8 μg per 10⁶ cells. In various embodiments, the cells are stimulated in the presence of or of about 0.8 μg per 10⁶ cells.

a. Bead Reagents

In certain embodiments, the stimulatory reagent contains a particle, e.g., a bead, that is conjugated or linked to one or more agents, e.g., biomolecules, that are capable of activating and/or expanding cells, e.g., T cells. In some embodiments, the one or more agents are bound to a solid support. In some embodiments, the solid support is or comprises a bead. In some embodiments, the one or more agents are bound to a bead. In some embodiments, the bead is biocompatible, i.e., composed of a material that is suitable for biological use. In some embodiments, the beads are non-toxic to cultured cells, e.g., cultured T cells. In some embodiments, the beads may be any particles which are capable of attaching agents in a manner that permits an interaction between the agent and a cell.

In some embodiments, a stimulatory reagent contains one or more agents that are capable of activating and/or expanding cells, e.g., T cells, that are bound to or otherwise attached to a bead, for example to the surface of the bead. In certain embodiments, the bead is a non-cell particle. In particular embodiments, the bead may include a colloidal particle, a microsphere, nanoparticle, a magnetic bead, or the like. In some embodiments the beads are agarose beads. In certain embodiments, the beads are sepharose beads.

In particular embodiments, the stimulatory reagent contains beads that are monodisperse. In certain embodiments, beads that are monodisperse comprise size dispersions having a diameter standard deviation of less than 5% from each other.

In some embodiments, the bead contains one or more agents, such as an agent that is coupled, conjugated, or linked (directly or indirectly) to the surface of the bead. In some embodiments, an agent as contemplated herein can include, but is not limited to, RNA, DNA, proteins (e.g., enzymes), antigens, polyclonal antibodies, monoclonal antibodies, antibody fragments, carbohydrates, lipids lectins, or any other biomolecule with an affinity for a desired target. In some embodiments, the desired target is a T cell receptor and/or a component of a T cell receptor. In certain embodiments, the desired target is CD3. In certain embodiment, the desired target is a T cell costimulatory molecule, e.g., CD28, CD137 (4-1-BB), OX40, or ICOS. The one or more agents may be attached directly or indirectly to the bead by a variety of methods known and available in the art. The attachment may be covalent, noncovalent, electrostatic, or hydrophobic and may be accomplished by a variety of attachment means, including for example, a chemical means, a mechanical means, or an enzymatic means. In some embodiments, a biomolecule (e.g., a biotinylated anti-CD3 antibody) may be attached indirectly to the bead via another biomolecule (e.g., anti-biotin antibody) that is directly attached to the bead.

In some embodiments, the stimulatory reagent contains a bead and one or more agents that directly interact with a macromolecule on the surface of a cell. In certain embodiments, the bead (e.g., a paramagnetic bead) interacts with a cell via one or more agents (e.g., an antibody) specific for one or more macromolecules on the cell (e.g., one or more cell surface proteins). In certain embodiments, the bead (e.g., a paramagnetic bead) is labeled with a first agent described herein, such as a primary antibody (e.g., an anti-biotin antibody) or other biomolecule, and then a second agent, such as a secondary antibody (e.g., a biotinylated anti-CD3 antibody) or other second biomolecule (e.g., streptavidin), is added, whereby the secondary antibody or other second biomolecule specifically binds to such primary antibodies or other biomolecule on the particle.

In some embodiments, the stimulatory reagent contains one or more agents (e.g., antibody) that is attached to a bead (e.g., a paramagnetic bead) and specifically binds to one or more of the following macromolecules on a cell (e.g., a T cell): CD2, CD3, CD4, CD5, CD8, CD25, CD27, CD28, CD29, CD31, CD44, CD45RA, CD45RO, CD54 (ICAM-1), CD127, MHCI, MHCII, CTLA-4, ICOS, PD-1, OX40, CD27L (CD70), 4-1BB (CD137), 4-1BBL, CD30L, LIGHT, IL-2R, IL-12R, IL-1R, IL-15R; IFN-gammaR, TNF-alphaR, IL-4R, IL-10R, CD18/CD11a (LFA-1, α_{L}β2), CD62L (L-selectin), CD29/CD49d (VLA-4), Notch ligand (e.g. Delta-like 1/4, Jagged 1/2, etc.), CCR1, CCR2, CCR3, CCR4, CCR5, CCR7, and CXCR3 or fragment thereof including the corresponding ligands to these macromolecules or fragments thereof. In some embodiments, an agent (e.g., antibody) attached to the bead specifically binds to one or more of the following macromolecules on a cell (e.g. a T cell): CD28, CD62L, CCR7, CD27, CD127, CD3, CD4, CD8, CD45RA, and/or CD45RO.

In some embodiments, one or more of the agents attached to the bead is an antibody. The antibody can include a polyclonal antibody, monoclonal antibody (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). In some embodiments, the stimulatory reagent is an antibody fragment (including antigen-binding fragment), e.g., a Fab, Fab′-SH, Fv, scFv, or (Fab′)2 fragment. It will be appreciated that constant regions of any isotype can be used for the antibodies contemplated herein, including IgG, IgM, IgA, IgD, and IgE constant regions, and that such constant regions can be obtained from any human or animal species (e.g., murine species). In some embodiments, the agent is an antibody that binds to and/or recognizes one or more components of a T cell receptor. In particular embodiments, the agent is an anti-CD3 antibody. In certain embodiments, the agent is an antibody that binds to and/or recognizes a co-receptor. In some embodiments, the stimulatory reagent comprises an anti-CD28 antibody. In some embodiments, the bead has a diameter of greater than at or about 0.001 μm, greater than at or about 0.01 μm, greater than at or about 0.1 μm, greater than at or about 1.0 μm, greater than at or about 10 μm, greater than at or about 50 μm, greater than at or about 100 μm, or greater than at or about 1000 μm and no more than at or about 1500 μm. In some embodiments, the bead has a diameter of at or about 1.0 μm to at or about 500 μm, at or about 1.0 μm to at or about 150 μm, at or about 1.0 μm to at or about 30 μm, at or about 1.0 μm to at or about 10 μm, at or about 1.0 μm to at or about 5.0 μm, at or about 2.0 μm to at or about 5.0 μm, or at or about 3.0 μm to at or about 5.0 μm. In some embodiments, the bead has a diameter of at or about 3 μm to at or about 5 μm. In some embodiments, the bead has a diameter of at least or at least about or about 0.001 μm, 0.01 μm, 0.1 μm, 0.5 μm, 1.0 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 12 μm, 14 μm, 16 μm, 18 μm, or 20 μm. In certain embodiments, the bead has a diameter of or about 4.5 μm. In certain embodiments, the bead has a diameter of or about 2.8 μm.

In some embodiments, the beads have a density of greater than at or about 0.001 g/cm³, greater than at or about 0.01 g/cm³, greater than at or about 0.05 g/cm³, greater than at or about 0.1 g/cm³, greater than at or about 0.5 g/cm³, greater than at or about 0.6 g/cm³, greater than at or about 0.7 g/cm³, greater than at or about 0.8 g/cm³, greater than at or about 0.9 g/cm³, greater than at or about 1 g/cm³, greater than at or about 1.1 g/cm³, greater than at or about 1.2 g/cm³, greater than at or about 1.3 g/cm³, greater than at or about 1.4 g/cm³, greater than at or about 1.5 g/cm³, greater than at or about 2 g/cm³, greater than at or about 3 g/cm³, greater than at or about 4 g/cm³, or greater than at or about 5 g/cm³. In some embodiments, the beads have a density of between at or about 0.001 g/cm³ and at or about 100 g/cm³, at or about 0.01 g/cm³ and at or about 50 g/cm³, at or about 0.1 g/cm³ and at or about 10 g/cm³, at or about 0.1 g/cm³ and at or about 0.5 g/cm³, at or about 0.5 g/cm³ and at or about 1 g/cm³, at or about 0.5 g/cm³ and at or about 1.5 g/cm³, at or about 1 g/cm³ and at or about 1.5 g/cm³, at or about 1 g/cm³ and at or about 2 g/cm³, or at or about 1 g/cm³ and at or about 5 g/cm³. In some embodiments, the beads have a density of at or about 0.5 g/cm³, at or about 0.5 g/cm³, at or about 0.6 g/cm³, at or about 0.7 g/cm³, at or about 0.8 g/cm³, at or about 0.9 g/cm³, at or about 1.0 g/cm³, at or about 1.1 g/cm³, at or about 1.2 g/cm³, at or about 1.3 g/cm³, at or about 1.4 g/cm³, at or about 1.5 g/cm³, at or about 1.6 g/cm³, at or about 1.7 g/cm³, at or about 1.8 g/cm³, at or about 1.9 g/cm³, or at or about 2.0 g/cm³. In certain embodiments, the beads have a density of at or about 1.6 g/cm³. In particular embodiments, the beads or particles have a density of at or about 1.5 g/cm³. In certain embodiments, the particles have a density of at or about 1.3 g/cm³.

In certain embodiments, a plurality of the beads has a uniform density. In certain embodiments, a uniform density comprises a density standard deviation of less than at or about 10%, less than at or about 5%, or less than at or about 1% of the mean bead density.

In some embodiments, the beads have a surface area of between at or about 0.001 m² per each gram of particles (m²/g) to at or about 1,000 m²/g, at or about 0.010 m²/g to at or about 100 m²/g, at or about 0.1 m²/g to at or about 10 m²/g, at or about 0.1 m²/g to at or about 1 m²/g, at or about 1 m²/g to at or about 10 m²/g, at or about 10 m²/g to at or about 100 m²/g, at or about 0.5 m²/g to at or about 20 m²/g, at or about 0.5 m²/g to at or about 5 m²/g, or at or about 1 m²/g to at or about 4 m²/g. In some embodiments, the particles or beads have a surface area of at or about 1 m²/g to at or about 4 m²/g.

In some embodiments, the bead contains at least one material at or near the bead surface that can be coupled, linked, or conjugated to an agent. In some embodiments, the bead is surface functionalized, i.e., comprises functional groups that are capable of forming a covalent bond with a binding molecule, e.g., a polynucleotide or a polypeptide. In particular embodiments, the bead comprises surface-exposed carboxyl, amino, hydroxyl, tosyl, epoxy, and/or chloromethyl groups. In particular embodiments, the beads comprise surface exposed agarose and/or sepharose. In certain embodiments, the bead surface comprises attached stimulatory reagents that can bind or attach binding molecules. In particular embodiments, the biomolecules are polypeptides. In some embodiments, the beads comprise surface exposed protein A, protein G, or biotin.

In some embodiments, the bead reacts in a magnetic field. In some embodiments, the bead is a magnetic bead. In some embodiments, the magnetic bead is paramagnetic. In particular embodiments, the magnetic bead is superparamagnetic. In certain embodiments, the beads do not display any magnetic properties unless they are exposed to a magnetic field.

In particular embodiments, the bead comprises a magnetic core, a paramagnetic core, or a superparamagnetic core. In some embodiments, the magnetic core contains a metal. In some embodiments, the metal can be, but is not limited to, iron, nickel, copper, cobalt, gadolinium, manganese, tantalum, zinc, zirconium or any combinations thereof. In certain embodiments, the magnetic core comprises metal oxides (e.g., iron oxides), ferrites (e.g., manganese ferrites, cobalt ferrites, nickel ferrites, etc.), hematite and metal alloys (e.g., CoTaZn). In some embodiments, the magnetic core comprises one or more of a ferrite, a metal, a metal alloy, an iron oxide, or chromium dioxide. In some embodiments, the magnetic core comprises elemental iron or a compound thereof. In some embodiments, the magnetic core comprises one or more of magnetite (Fe₃O₄), maghemite (γFe₂O₃), or greigite (Fe₃S₄). In some embodiments, the inner core comprises an iron oxide (e.g., Fe₃O₄).

In certain embodiments, the bead contains a magnetic, paramagnetic, and/or superparamagnetic core that is covered by a surface functionalized coat or coating. In some embodiments, the coat can contain a material that can include, but is not limited to, a polymer, a polysaccharide, a silica, a fatty acid, a protein, a carbon, agarose, sepharose, or a combination thereof. In some embodiments, the polymer can be a polyethylene glycol, poly (lactic-co-glycolic acid), polyglutaraldehyde, polyurethane, polystyrene, or a polyvinyl alcohol. In certain embodiments, the outer coat or coating comprises polystyrene. In particular embodiments, the outer coating is surface functionalized.

In some embodiments, the stimulatory reagent comprises a bead that contains a metal oxide core (e.g., an iron oxide core) and a coat, wherein the metal oxide core comprises at least one polysaccharide (e.g., dextran), and wherein the coat comprises at least one polysaccharide (e.g., amino dextran), at least one polymer (e.g., polyurethane) and silica. In some embodiments the metal oxide core is a colloidal iron oxide core. In certain embodiments, the one or more agents include an antibody or antigen-binding fragment thereof. In particular embodiments, the one or more agents include an anti-CD3 antibody and an anti-CD28 antibody. In some embodiments, the stimulatory reagent comprises an anti-CD3 antibody, anti-CD28 antibody, and an anti-biotin antibody. In some embodiments, the stimulatory reagent comprises an anti-biotin antibody. In some embodiments, the bead has a diameter of about 3 μm to about 10 μm. In some embodiments, the bead has a diameter of about 3 μm to about 5 μm. In certain embodiments, the bead has a diameter of about 3.5 μm.

In some embodiments, the stimulatory reagent comprises one or more agents that are attached to a bead comprising a metal oxide core (e.g., an iron oxide inner core) and a coat (e.g., a protective coat), wherein the coat comprises polystyrene. In certain embodiments, the beads are monodisperse, paramagnetic (e.g., superparamagnetic) beads comprising a paramagnetic (e.g., superparamagnetic) iron core, e.g., a core comprising magnetite (Fe₃O₄) and/or maghemite (γFe₂O₃) c and a polystyrene coat or coating. In some embodiments, the bead is non-porous. In some embodiments, the beads contain a functionalized surface to which the one or more agents are attached. In certain embodiments, the one or more agents are covalently bound to the beads at the surface. In some embodiments, the one or more agents include an antibody or antigen-binding fragment thereof. In some embodiments, the one or more agents include an anti-CD3 antibody and an anti-CD28 antibody. In some embodiments, the one or more agents include an anti-CD3 antibody and/or an anti-CD28 antibody, and an antibody or antigen fragment thereof capable of binding to a labeled antibody (e.g., biotinylated antibody), such as a labeled anti-CD3 or anti-CD28 antibody. In certain embodiments, the beads have a density of about 1.5 g/cm³ and a surface area of about 1 m²/g to about 4 m²/g. In particular embodiments, the beads are monodisperse superparamagnetic beads that have a diameter of about 4.5 μm and a density of about 1.5 g/cm³. In some embodiments, the beads the beads are monodisperse superparamagnetic beads that have a mean diameter of about 2.8 μm and a density of about 1.3 g/cm³.

In some embodiments, the composition of enriched T cells is incubated with stimulatory reagent a ratio of beads to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of beads to cells is between 2.5:1 and 0.2:1, between 2:1 and 0.5:1, between 1.5:1 and 0.75:1, between 1.25:1 and 0.8:1, or between 1.1:1 and 0.9:1. In particular embodiments, the ratio of beads to cells is about 1:1 or is 1:1.

b. Oligomeric Reagents

In particular embodiments, the stimulatory reagent contains an oligomeric reagent, e.g., a streptavidin mutein reagent, that is conjugated, linked, or attached to one or more agent, e.g., ligand, which is capable of activating an intracellular signaling domain of a TCR complex. In some embodiments, the one or more agents have an attached binding domain or binding partner (e.g., a binding partner C) that is capable of binding to oligomeric reagent at a particular binding sites (e.g., binding site Z). In some embodiments, a plurality of the agent is reversibly bound to the oligomeric reagent. In various embodiments, the oligomeric reagent has a plurality of the particular binding sites which, in certain embodiments, are reversibly bound to a plurality of agents at the binding domain (e.g., binding partner C). In some embodiments, the amount of bound agents are reduced or decreased in the presence of a competition reagent, e.g., a reagent that is also capable of binding to the particular binding sites (e.g., binding site Z). Among oligomeric stimulatory reagents, including anti-CD3/anti-CD28 oligomeric streptavidin mutiein reagent, are described in International PCT publication NO. WO2018/197949.

In some embodiments, the stimulatory reagent is or includes a reversible systems in which at least one agent (e.g., an agent that is capable of producing a signal in a cell such as a T cell) is associated, e.g., reversibly associated, with the oligomeric reagent. In some embodiments, the reagent contains a plurality of binding sites capable of binding, e.g., reversibly binding, to the agent. In some embodiments, the stimulatory reagent is reversibly bound on the surface of an oligomeric particle reagent comprising a plurality of streptavidin or streptavidin mutein molecules. In some embodiments, the stimulatory reagent (e.g., the primary agent and secondary agent) are reversibly bound on the surface of an oligomeric particle reagent comprising a plurality of streptavidin or streptavidin mutein molecules. In some cases, the reagent is a oligomeric particle reagent having at least one attached agent capable of producing a signal in a cell such as a T cell. In some embodiments, the agent contains at least one binding site, e.g., a binding site B, that can specifically bind an epitope or region of the molecule and also contains a binding partner, also referred to herein as a binding partner C, that specifically binds to at least one binding site of the reagent, e.g., binding site Z of the reagent. In some embodiments, the binding interaction between the binding partner C and the at least one binding site Z is a non-covalent interaction. In some cases, the binding interaction between the binding partner C and the at least one binding site Z is a covalent interaction. In some embodiments, the binding interaction, such as non-covalent interaction, between the binding partner C and the at least one binding site Z is reversible.

Substances that may be used as oligomeric reagents in such reversible systems are known, see e.g., U.S. Pat. Nos. 5,168,049; 5,506,121; 6,103,493; 7,776,562; 7,981,632; 8,298,782; 8,735,540; 9,023,604; and International published PCT Appl. Nos. WO2013/124474 and WO2014/076277. Non-limiting examples of reagents and binding partners capable of forming a reversible interaction, as well as substances (e.g., competition reagents) capable of reversing such binding, are described below.

In some embodiments, the oligomeric reagent is an oligomer of streptavidin, streptavidin mutein or analog, avidin, an avidin mutein or analog (such as neutravidin) or a mixture thereof, in which such oligomeric reagent contains one or more binding sites for reversible association with the binding domain of the agent (e.g., a binding partner C). In some embodiments, the binding domain of the agent can be a biotin, a biotin derivative or analog, or a streptavidin-binding peptide or other molecule that is able to specifically bind to streptavidin, a streptavidin mutein or analog, avidin or an avidin mutein or analog.

In certain embodiments, one or more agents (e.g., agents that are capable of producing a signal in a cell such as a T cell) associate with, such as are reversibly bound to, the oligomeric reagent, such as via the plurality of the particular binding sites (e.g., binding sites Z) present on the oligomeric reagent. In some cases, this results in the agents being closely arranged to each other such that an avidity effect can take place if a target cell having (at least two copies of) a cell surface molecule that is bound by or recognized by the agent is brought into contact with the agent.

In some embodiments, the oligomeric reagent is a streptavidin oligomer, a streptavidin mutein oligomer, a streptavidin analog oligomer, an avidin oligomer, an oligomer composed of avidin mutein or avidin analog (such as neutravidin) or a mixture thereof. In particular embodiments, the oligomeric reagents contain particular binding sites that are capable of binding to a binding domain (e.g., the binding partner C) of an agent. In some embodiments, the binding domain can be a biotin, a biotin derivative or analog, or a streptavidin-binding peptide or other molecule that is able to specifically bind to streptavidin, a streptavidin mutein or analog, avidin or an avidin mutein or analog.

In some embodiments, the streptavidin can be wild-type streptavidin, or streptavidin muteins or analogs, such as streptavidin-like polypeptides. Likewise, avidin, in some aspects, includes wild-type avidin or muteins or analogs of avidin such as neutravidin, a deglycosylated avidin with modified arginines that typically exhibits a more neutral pi and is available as an alternative to native avidin. Generally, deglycosylated, neutral forms of avidin include those commercially available forms such as “Extravidin” available through Sigma Aldrich, or “NeutrAvidin” available from Thermo Scientific or Invitrogen, for example.

In some embodiments, the reagent is a streptavidin or a streptavidin mutein or analog. In some embodiments, wild-type streptavidin (wt-streptavidin) has the amino acid sequence disclosed by Argarana et al, Nucleic Acids Res. 14 (1986) 1871-1882 (SEQ ID NO: 34). In general, streptavidin naturally occurs as a tetramer of four identical subunits, i.e. it is a homo-tetramer, where each subunit contains a single binding site for biotin, a biotin derivative or analog or a biotin mimic. An exemplary sequence of a streptavidin subunit is the sequence of amino acids set forth in SEQ ID NO: 34, but such a sequence also can include a sequence present in homologs thereof from other Streptomyces species. In particular, each subunit of streptavidin may exhibit a strong binding affinity for biotin with an equilibrium dissociation constant (K_{D}) on the order of at or about 10⁻¹⁴ M. In some cases, streptavidin can exist as a monovalent tetramer in which only one of the four binding sites is functional (Howarth et al. (2006) Nat. Methods, 3:267-73; Zhang et al. (2015) Biochem. Biophys. Res. Commun., 463:1059-63)), a divalent tetramer in which two of the four binding sites are functional (Fairhead et al. (2013) J. Mol. Biol., 426:199-214), or can be present in monomeric or dimeric form (Wu et al. (2005) J. Biol. Chem., 280:23225-31; Lim et al. (2010) Biochemistry, 50:8682-91).

In some embodiments, streptavidin may be in any form, such as wild-type or unmodified streptavidin, such as a streptavidin from a Streptomyces species or a functionally active fragment thereof that includes at least one functional subunit containing a binding site for biotin, a biotin derivative or analog or a biotin mimic, such as generally contains at least one functional subunit of a wild-type streptavidin from Streptomyces avidinii set forth in SEQ ID NO: 34 or a functionally active fragment thereof. For example, in some embodiments, streptavidin can include a fragment of wild-type streptavidin, which is shortened at the N- and/or C-terminus. Such minimal streptavidins include any that begin N-terminally in the region of amino acid positions 10 to 16 of SEQ ID NO: 34 and terminate C-terminally in the region of amino acid positions 133 to 142 of SEQ ID NO: 34. In some embodiments, a functionally active fragment of streptavidin contains the sequence of amino acids set forth in SEQ ID NO: 35. In some embodiments, streptavidin, such as set forth in SEQ ID NO: 35, can further contain an N-terminal methionine at a position corresponding to Ala13 with numbering set forth in SEQ ID NO: 34. Reference to the position of residues in streptavidin or streptavidin muteins is with reference to numbering of residues in SEQ ID NO: 34.

Examples of streptavidins or streptavidin muteins are mentioned, for example, in WO 86/02077, DE 19641876 A1, U.S. Pat. No. 6,022,951, WO 98/40396 or WO 96/24606. Examples of streptavidin muteins are known in the art, see e.g., U.S. Pat. Nos. 5,168,049; 5,506,121; 6,022,951; 6,156,493; 6,165,750; 6,103,493; or 6,368,813; or International published PCT App. No. WO2014/076277.

In some embodiments, a streptavidin mutein can contain amino acids that are not part of an unmodified or wild-type streptavidin or can include only a part of a wild-type or unmodified streptavidin. In some embodiments, a streptavidin mutein contains at least one subunit that can have one more amino acid substitutions (replacements) compared to a subunit of an unmodified or wild-type streptavidin, such as compared to the wild-type streptavidin subunit set forth in SEQ ID NO: 34 or a functionally active fragment thereof, e.g. set forth in SEQ ID NO: 35 or SEQ ID NO: 56.

In some embodiments, the binding affinity, such as dissociation constant (K_{d}), of streptavidin or a streptavidin mutein for a binding domain is less than at or about 1×10⁻⁴ M, 5×10⁻⁴ M, 1×10⁻⁵ M, 5×10⁻⁵ M, 1×10⁻⁶ M, 5×10⁻⁶ M or 1×10⁻⁷ M, but generally greater than 1×10⁻¹³ M, 1×10⁻¹² M or 1×10⁻¹¹ M. For example, peptide sequences (Strep-tags), such as disclosed in U.S. Pat. No. 5,506,121, can act as biotin mimics and demonstrate a binding affinity for streptavidin, e.g., with a K_{D} of approximately between 10⁴ and 10⁻⁵ M. In some cases, the binding affinity can be further improved by making a mutation within the streptavidin molecule, see e.g., U.S. Pat. No. 6,103,493 or International published PCT App. No. WO2014/076277. In some embodiments, binding affinity can be determined by methods known in the art, such as any described herein.

In some embodiments, the reagent, such as a streptavidin or streptavidin mutein, exhibits binding affinity for a peptide ligand binding partner, which peptide ligand binding partner can be the binding partner C present in the agent (e.g., receptor-binding agent or selection agent). In some embodiments, the peptide sequence contains a sequence with the general formula His-Pro-Xaa, where Xaa is glutamine, asparagine, or methionine, such as the sequence set forth in SEQ ID NO: 51. In some embodiments, the peptide sequence has the general formula set forth in SEQ ID NO: 52, such as set forth in SEQ ID NO: 42. In one example, the peptide sequence is Trp-Arg-His-Pro-Gln-Phe-Gly-Gly (also called Strep-Tag®, set forth in SEQ ID NO: 43). In one example, the peptide sequence is Trp-Ser-His-Pro-Gln-Phe-Glu-Lys (also called Strep-Tag® II, set forth in SEQ ID NO: 37). In some embodiments, the peptide ligand contains a sequential arrangement of at least two streptavidin-binding modules, wherein the distance between the two modules is at least 0 and not greater than 50 amino acids, wherein one binding module has 3 to 8 amino acids and contains at least the sequence His-Pro-Xaa, where Xaa is glutamine, asparagine, or methionine, and wherein the other binding module has the same or different streptavidin peptide ligand, such as set forth in SEQ ID NO: 52 (see e.g., International Published PCT Appl. No. WO02/077018; U.S. Pat. No. 7,981,632). In some embodiments, the peptide ligand contains a sequence having the formula set forth in any of SEQ ID NO: 44 or 45. In some embodiments, the peptide ligand has the sequence of amino acids set forth in any of SEQ ID NOS: 38-40, 46, and 47. In most cases, all these streptavidin binding peptides bind to the same binding site, namely the biotin binding site of streptavidin. If one or more of such streptavidin binding peptides is used as binding partners C, e.g., C1 and C2, the multimerization reagent and/or oligomeric particle reagents bound to the one or more agents via the binding partner C is typically composed of one or more streptavidin muteins.

In some embodiments, the streptavidin mutein is a mutant as described in U.S. Pat. No. 6,103,493. In some embodiments, the streptavidin mutein contains at least one mutation within the region of amino acid positions 44 to 53, based on the amino acid sequence of wild-type streptavidin, such as set forth in SEQ ID NO: 34. In some embodiments, the streptavidin mutein contains a mutation at one or more residues 44, 45, 46, and/or 47. In some embodiments, the streptavidin mutein contains a replacement of Glu at position 44 of wild-type streptavidin with a hydrophobic aliphatic amino acid, e.g., Val, Ala, Ile or Leu, any amino acid at position 45, an aliphatic amino acid, such as a hydrophobic aliphatic amino acid at position 46 and/or a replacement of Val at position 47 with a basic amino acid, e.g. Arg or Lys, such as generally Arg. In some embodiments, Ala is at position 46 and/or Arg is at position 47 and/or Val or Ile is at position 44. In some embodiments, the streptavidin mutant contains residues Val44-Thr45-Ala46-Arg47, such as set forth in exemplary streptavidin muteins containing the sequence of amino acids set forth in SEQ ID NO: 48 or SEQ ID NO: 49 or 50 (also known as streptavidin mutant 1, SAM1). In some embodiments, the streptavidin mutein contains residues Ile44-Gly45-Ala46-Arg47, such as set forth in exemplary streptavidin muteins containing the sequence of amino acids set forth in SEQ ID NO: 53, 36, or 41 (also known as SAM2). In some cases, such streptavidin mutein are described, for example, in U.S. Pat. No. 6,103,493, and are commercially available under the trademark Strep-Tactin®. In some embodiments, the mutein streptavidin contains the sequence of amino acids set forth in SEQ ID NO: 54 or SEQ ID NO: 55. In particular embodiments, the molecule is a tetramer of streptavidin or a streptavidin mutein comprising a sequence set forth in any of SEQ ID NOS: 35, 49, 36, 54, 56, 50, or 41, which, as a tetramer, is a molecule that contains 20 primary amines, including 1 N-terminal amine and 4 lysines per monomer.

In some embodiments, streptavidin mutein exhibits a binding affinity characterized by an equilibrium dissociation constant (K_{D}) that is or is less than at or about 3.7×10⁻⁵ M for the peptide ligand (Trp-Arg-His-Pro-Gln-Phe-Gly-Gly; also called Strep-Tag®, set forth in SEQ ID NO: 43) and/or that is or is less than at or about 7.1×10⁻⁵ M for the peptide ligand (Trp-Ser-His-Pro-Gln-Phe-Glu-Lys; also called Strep-Tag® II, set forth in SEQ ID NO: 37) and/or that is or is less than at or about 7.0×10⁻⁵ M, 5.0×10⁻⁵ M, 1.0×10⁻⁵ M, 5.0×10⁻⁶ M, 1.0×10⁻⁶ M, 5.0×10⁻⁷ M, or 1.0×10⁻⁷ M, but generally greater than at or about 1×10⁻¹³ M, 1×10⁻¹² M or 1×10⁻¹¹ M for any of the peptide ligands set forth in any of SEQ ID NOS: 37, 44-47, 38-40, 42, 43, 51, and 52.

In some embodiments, the resulting streptavidin mutein exhibits a binding affinity characterized by an equilibrium association constant (KA) that is or is greater than at or about 2.7×10⁴ M⁻¹ for the peptide ligand (Trp-Arg-His-Pro-Gln-Phe-Gly-Gly; also called Strep-Tag®, set forth in SEQ ID NO: 43) and/or that is or is greater than at or about 1.4×10⁴ M⁻¹ for the peptide ligand (Trp-Ser-His-Pro-Gln-Phe-Glu-Lys; also called Strep-Tag® II, set forth in SEQ ID NO: 37) and/or that is or is greater than at or about 1.43×10⁴ M⁻¹, 1.67×10⁴ M⁻¹, 2×10⁴ M⁻¹, 3.33×10⁴ M⁻¹, 5×10⁴ M⁻¹, 1×10⁵ M⁻¹, 1.11×10⁵ M⁻¹, 1.25×10⁵ M⁻¹, 1.43×10⁵ M⁻¹, 1.67×10⁵ M⁻¹, 2×10⁵ M⁻¹, 3.33×10⁵ M⁻¹, 5×10⁵ M⁻¹, 1×10⁶ M⁻¹, 1.11×10⁶ M⁻¹, 1.25×10⁶ M⁻¹, 1.43×10⁶ M⁻¹, 1.67×10⁶ M⁻¹, 2×10⁶ M⁻¹, 3.33×10⁶ M⁻¹, 5×10⁶ M⁻¹, 1×10⁷ M⁻¹, but generally less than 1×10¹³ M⁻¹, 1×10¹² M⁻¹ or 1×10¹¹ M⁻¹ for any of the peptide ligands set forth in any of SEQ ID NOS: 37, 44-47, 38-40, 42, 43, 51, and 52.

In particular embodiments, provided herein is an oligomeric particle reagent that is composed of and/or contains a plurality of streptavidin or streptavidin mutein tetramers. In certain embodiments, the oligomeric particle reagent provided herein contains a plurality of binding sites that reversibly bind or are capable of reversibly binding to one or more agents, e.g., a stimulatory agent and/or a selection agent. In some embodiments, the oligomeric particle has a radius, e.g., an average or mean radius, of between at or about 70 nm and at or about 125 nm, inclusive; a molecular weight of between at or about 1×10⁷ g/mol and at or about 1×10⁹ g/mol, inclusive; and/or between at or about 1,000 and at or about 5,000 streptavidin or streptavidin mutein tetramers, inclusive. In some embodiments, the oligomeric particle reagent is bound, e.g., reversibly bound, to one or more agents such as an agent that binds to a molecule, e.g. receptor, on the surface of a cell. In certain embodiments, the one or more agents are or comprise an antibody or antigen binding fragment thereof, such as a Fab. In some embodiments, the one or more agents specifically bind to one or more of the following macromolecules on a cell (e.g., a T cell): CD2, CD3, CD4, CD5, CD8, CD25, CD27, CD28, CD29, CD31, CD44, CD45RA, CD45RO, CD54 (ICAM-1), CD127, MHCI, MHCII, CTLA-4, ICOS, PD-1, OX40, CD27L (CD70), 4-1BB (CD137), 4-1BBL, CD30L, LIGHT, IL-2R, IL-12R, IL-1R, IL-15R; IFN-gammaR, TNF-alphaR, IL-4R, IL-10R, CD18/CD11a (LFA-1), CD62L (L-selectin), CD29/CD49d (VLA-4), Notch ligand (e.g. Delta-like 1/4, Jagged 1/2, etc.), CCR1, CCR2, CCR3, CCR4, CCR5, CCR7, and CXCR3 or fragment thereof including the corresponding ligands to these macromolecules or fragments thereof. In some embodiments, the one or more agents specifically bind to one or more of the following macromolecules on a cell (e.g., a T cell): CD28, CD62L, CCR7, CD27, CD127, CD3, CD4, CD8, CD45RA, and/or CD45RO. In some embodiments, the one or more agent comprises an antibody or antigen binding fragment thereof, such as a Fab, and the antibody can include a polyclonal antibody, monoclonal antibody (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). In some embodiments, the one or more reagent is or comprises an antibody fragment (including antigen-binding fragment), e.g., a Fab, Fab′-SH, Fv, scFv, or (Fab′)2 fragment. It will be appreciated that constant regions of any isotype can be used for the antibodies contemplated herein, including IgG, IgM, IgA, IgD, and IgE constant regions, and that such constant regions can be obtained from any human or animal species (e.g., murine species). In some embodiments, the one or more reagent is or comprises an antibody that binds to and/or recognizes one or more components of a T cell receptor. In particular embodiments, the one or more reagent is or comprises an anti-CD3 antibody. In certain embodiments, the one or more reagent is or comprises an antibody that binds to and/or recognizes a co-receptor. In some embodiments, the one or more reagent is or comprises an anti-CD28 antibody. In some embodiments, the one or more reagent is or comprises an anti-CD3 and/or an anti-CD28 antibody or antigen binding fragment thereof, such as an antibody or antigen fragment thereof that contains a binding partner, e.g., a streptavidin binding peptide, e.g. Strep-Tag® II. In particular embodiments, the one or more agent is or comprises an anti-CD3 and/or an anti-CD28 Fab containing a binding partner, e.g., a streptavidin binding peptide, e.g. Strep-Tag® II.

In some embodiments, provided herein is an oligomeric particle reagent that is composed of and/or contains a plurality of streptavidin or streptavidin mutein tetramers. In certain embodiments, the oligomeric particle reagent provided herein contains a plurality of binding sites that reversibly bind or are capable of reversibly binding to one or more agents, e.g., a stimulatory agent and/or a selection agent. In some embodiments, the oligomeric particle has a radius, e.g., an average radius, of between at or about 80 nm and at or about 120 nm, inclusive; a molecular weight, e.g., an average molecular weight of between at or about 7.5×10⁶ g/mol and at or about 2×10⁸ g/mol, inclusive; and/or an amount, e.g., an average amount, of between at or about 500 and at or about 10,000 streptavidin or streptavidin mutein tetramers, inclusive. In some embodiments, the oligomeric particle reagent is bound, e.g., reversibly bound, to one or more agents, such as an agent that binds to a molecule, e.g. receptor, on the surface of a cell. In some embodiments, the agent is an anti-CD3 and/or an anti-CD28 Fab, such as a Fab that contains a binding partner, e.g., a streptavidin binding peptide, e.g. Strep-Tag® II. In particular embodiments, the one or more agents is an anti-CD3 and/or an anti CD28 Fab containing a binding partner, e.g., a streptavidin binding peptide, e.g. Strep-Tag® II.

In some embodiments, the cells are stimulated in the presence of, of about, or of at least at or about 0.01 μg, 0.02 μg, 0.03 μg, 0.04 μg, 0.05 μg, 0.1 μg, 0.2 μg, 0.3 μg, 0.4 μg, 0.5 μg, 0.75 μg, 1 μg, 2 μg, 3 μg, 4 μg, 5 μg, 6 μg, 7 μg, 8 μg, 9 μg, or 10 μg of the oligomeric stimulatory reagent per 10⁶ cells. In some embodiments, the cells are stimulated in the presence of or of about 4 μg per 10⁶ cells. In particular embodiments, the cells are stimulated in the presence of or of about 0.8 μg per 10⁶ cells. In certain aspects, 4 μg of the oligomeric stimulatory reagent is or includes at or about 3 μg of oligomeric particles and at or about 1 μg of attached agents, e.g., at or about 0.5 μg of anti-CD3 Fabs and at or about 0.5 μg of anti-CD28 Fabs.

2 Removal of the Stimulatory Reagent from Cells

In some embodiments, the stimulatory reagent is removed or separated from the cells or cell populations prior to collecting, harvesting, or formulating the cells. In some embodiments, the stimulatory reagents are removed or separated from the cells or cell populations after or during the incubation, e.g., an incubation described herein such as in Section I-D. In certain embodiments, the cells or cell population undergoes a process, procedure, step, or technique to remove the stimulatory reagent after the incubation but prior to steps for collecting, harvesting, or formulating the cells. In particular embodiments, the cells or cell population undergoes a process, procedure, step, or technique to remove the stimulatory reagent after the incubation. In some aspects, when stimulatory reagent is separated or removed from the cells during the incubation, the cells are returned to the same incubation conditions as prior to the separation or removal for the remaining duration of the incubation.

In certain embodiments, the stimulatory reagent is removed and/or separated from the cells. In particular embodiments, the binding and/or association between a stimulatory reagent and cells may, in some circumstances, be reduced over time during the incubation. In certain embodiments, one or more agents may be added to reduce the binding and/or association between the stimulatory reagent and the cells. In particular embodiments, a change in cell culture conditions, e.g., the addition of an agent and/or a change in media temperature and/or pH, may reduce the binding and/or association between the stimulatory reagent and the cells. Thus, in some embodiments, the stimulatory reagent may be removed from an incubation, cell culture system, and/or a solution separately from the cells, e.g., without removing the cells from the incubation, cell culture system, and/or a solution as well.

In certain embodiments, the stimulatory reagent is separated and/or removed from the cells after an amount of time. In particular embodiments, the amount of time is an amount of time from the initiation of the stimulation. In particular embodiments the start of the incubation is considered at or at about the time the cells are contacted with the stimulatory reagent and/or a media or solution containing the stimulatory reagent. In particular embodiments, the stimulatory reagent is removed or separated from the cells within or within about 120 hours, 108 hours, 96 hours, 84 hours, 72 hours, 60 hours, 48 hours, 36 hours, 24 hours, or 12 hours, inclusive, of the initiation of the stimulation. In particular embodiments, the stimulatory reagent is removed or separated from the cells at or at about 48 hours after the stimulation is initiated. In certain embodiments, the stimulatory reagent is removed or separated from the cells at or at about 72 hours after the stimulation is initiated. In some embodiments, the stimulatory reagent is removed or separated from the cells at or at about 96 hours after the stimulation is initiated.

Methods for removing stimulatory reagents (e.g., stimulatory reagents that are or contain particles such as bead particles or magnetizable particles) from cells are known. In certain embodiments, a bead stimulatory reagent, e.g., an anti-CD3/anti-CD28 antibody conjugated paramagnetic bead, is separated or removed from the cells or the cell population. In some embodiments, the use of competing antibodies, such as non-labeled antibodies, can be used, which, for example, bind to a primary antibody of the stimulatory reagent and alter its affinity for its antigen on the cell, thereby permitting for gentle detachment. In some cases, after detachment, the competing antibodies may remain associated with the particle (e.g., bead particle) while the unreacted antibody is or may be washed away and the cell is free of isolating, selecting, enriching and/or activating antibody. Exemplary of such a reagent is DETACaBEAD (Friedl et al. 1995; Entschladen et al. 1997). In some embodiments, particles (e.g., bead particles) can be removed in the presence of a cleavable linker (e.g., DNA linker), whereby the particle-bound antibodies are conjugated to the linker (e.g., CELLection, Dynal). In some cases, the linker region provides a cleavable site to remove the particles (e.g., bead particles) from the cells after isolation, for example, by the addition of DNase or other releasing buffer. In some embodiments, other enzymatic methods can also be employed for release of a particle (e.g., bead particle) from cells. In some embodiments, the particles (e.g., bead particles or magnetizable particles) are biodegradable.

In some embodiments, the stimulatory reagent is magnetic, paramagnetic, and/or superparamagnetic, and/or contains a bead that is magnetic, paramagnetic, and/or superparamagnetic, and the stimulatory reagent may be removed from the cells by exposing the cells to a magnetic field. Examples of suitable equipment containing magnets for generating the magnetic field include DynaMag CTS (Thermo Fisher), Magnetic Separator (Takara) and EasySep Magnet (Stem Cell Technologies).

In particular embodiments, the stimulatory reagent is removed or separated from the cells prior to the completion of the provided methods, e.g., prior to harvesting, collecting, and/or formulating engineered cells produced by the methods provided herein. In some embodiments, the stimulatory reagent is removed and/or separated from the cells after engineering, e.g., transducing or transfecting, the cells. In certain embodiments, the stimulatory reagent is removed after the cultivation of the cells, e.g., prior to the cultivation of the engineered, e.g., transfected or transduced, cells under conditions to promote proliferation and/or expansion. In particular embodiments, the stimulatory reagent is removed after the cells achieve a threshold number, density, and/or expansion during the cultivation of the cells. In some embodiments, the stimulatory reagent is removed prior to formulating the cells, e.g., prior to forming the cultivated cells, such as cultivated cells that had achieved the threshold number, concentration, or expansion.

In some embodiments, the stimulatory bead reagent, e.g., the stimulatory magnetic bead reagent, is removed or separated from the cells or cell populations prior to collecting, harvesting, or formulating the cells. In some embodiments, the stimulatory bead reagent, e.g., the stimulatory magnetic bead reagent, are removed or separated from the cells or cell populations by exposure to a magnetic field during or after the incubation, e.g., an incubation described herein such as in Section I-D. In certain embodiments, the cells or cell population are exposed to the magnetic field to remove the stimulatory bead reagent, e.g., the stimulatory magnetic bead reagent, after the incubation but prior to steps for collecting, harvesting, or formulating the cells. In particular embodiments, the cells or cell population undergoes is exposed to the magnetic field to remove the stimulatory bead reagent, e.g., the stimulatory magnetic bead reagent, after the incubation. In some aspects, when the stimulatory bead reagent is separated or removed from the cells or cell population during the incubation, the cells or cell population are returned to the same incubation conditions as prior to the exposure to the magnetic field for the remaining duration of the incubation.

In particular embodiments, the stimulatory bead reagent, e.g., the stimulatory magnetic bead reagent, is removed or separated from the cells, e.g., by exposure to a magnetic field, within or within about 120 hours, 108 hours, 96 hours, 84 hours, 72 hours, 60 hours, 48 hours, 36 hours, 24 hours, or 12 hours, inclusive, of the initiation of the stimulation. In certain embodiments, the stimulatory bead reagent, e.g., the stimulatory magnetic bead reagent, is removed or separated from the cells, e.g., by exposure to a magnetic field, at or at about 72 hours after the stimulation is initiated. In some embodiments, the stimulatory bead reagent, e.g., the stimulatory magnetic bead reagent, is removed or separated from the cells, e.g., by exposure to a magnetic field, at or at about 96 hours after the stimulation is initiated.

In certain embodiments, the stimulatory reagent is separated and/or removed from the cells after an amount of time. In particular embodiments, the amount of time is an amount of time from the start and/or initiation of the incubation under stimulating conditions. In particular embodiments the start of the incubation is considered at or at about the time the cells are contacted with the stimulatory reagent and/or a media or solution containing the stimulatory reagent. In particular embodiments, the stimulatory reagent is removed or separated from the cells within or within about 28 days, 21 days, 20 days, 19 days, 18 days, 17 days, 16 days, 15 days, 14 days, 13 days, 12 days, 11 days, 10 days, or 9 days after the start or initiation of the incubation. In some embodiments, the stimulatory reagent is removed or separated from the cells within or within about 28 days, 21 days, 20 days, 19 days, 18 days, 17 days, 16 days, 15 days, 14 days, 13 days, 12 days, 11 days, 10 days, or 9 days after the CD4+ T cells and CD8+ T cells are pooled, combined, and/or mixed into the input composition. In certain embodiments, the stimulatory reagent is removed or separated from the cells within or within about 28 days, 21 days, 20 days, 19 days, 18 days, 17 days, 16 days, 15 days, 14 days, 13 days, 12 days, 11 days, 10 days, or 9 days after the CD4+ T cells and CD8+ T cells are obtained, isolated, enriched, and/or selected from a biological sample.

In some embodiments, removal of a stimulatory agent, such as an oligomeric stimulatory reagent as described, includes adding to the population of incubated T cells a substance, such as a competition agent, to disrupt, such as to lessen and/or terminate, the signaling of the stimulatory agent or agents. In some embodiments, the population of the incubated T cells contains the presence of a substance, such as a competition agent, e.g., biotin or a biotin analog, e.g., D-Biotin. In some embodiments, the substance, such as a competition agent, e.g., biotin or a biotin analog, e.g., D-Biotin, is present in an amount that is at least 1.5-fold greater, at least 2-fold, at least 3-fold, at least 4-fold, at least 5-fold, at least 10-fold, at least 100-fold, or at least 1000-fold or more greater than the amount of the substance in a reference population or preparation of cultured T cells in which the substance was not added exogenously during the incubation. In some embodiments, the amount of the substance, such as a competition agent, e.g., biotin or a biotin analog, e.g., D-Biotin, in the population of cultured T cells is from at or about 10 μM to at or about 100 μM, at or about 100 μM to at or about 1 mM, at or about 100 μM to at or about 500 μM, or at or about 10 μM to at or about 100 μM. In some embodiments, 10 μM or about 10 μM of biotin or a biotin analog, e.g., D-biotin, is added to the cells or the cell population to separate or remove the oligomeric stimulatory reagent from the cells or cell population.

In certain embodiments, the one or more agents (e.g., agents that stimulate or activate a TCR and/or a coreceptor) associate with, such as are reversibly bound to, the oligomeric reagent, such as via the plurality of the particular binding sites (e.g., binding sites Z) present on the oligomeric reagent. In some cases, this results in the agents being closely arranged to each other such that an avidity effect can take place if a target cell having (at least two copies of) a cell surface molecule that is bound by or recognized by the agent is brought into contact with the agent. In some aspects, the receptor binding reagent has a low affinity towards the receptor molecule of the cell at binding site B, such that the receptor binding reagent dissociates from the cell in the presence of the competition reagent. Thus, in some embodiments, the agents are removed from the cells in the presence of the competition reagent.

In some embodiments, the oligomeric stimulatory reagent is a streptavidin mutein oligomer with reversibly attached anti-CD3 and anti-CD28 Fabs. In some embodiments, the Fabs are attached contain streptavidin binding domains, e.g., that allow for the reversible attachment to the streptavidin mutein oligomer. In some cases, anti-CD3 and anti-CD28 Fabs are closely arranged to each other such that an avidity effect can take place if a T cell expressing CD3 and/or CD28 is brought into contact with the oligomeric stimulatory reagent with the reversibly attached Fabs. In some aspects, the Fabs have a low affinity towards CD3 and CD28, such that the Fabs dissociate from the cell in the presence of the competition reagent, e.g., biotin or a biotin variant or analogue. Thus, in some embodiments, the Fabs are removed or dissociated from the cells in the presence of the competition reagent, e.g., D-biotin.

In some embodiments, the stimulatory oligomeric reagent, e.g., the stimulatory oligomeric streptavidin mutein reagent, is removed or separated from the cells or cell populations prior to collecting, harvesting, or formulating the cells. In some embodiments, stimulatory oligomeric reagent, e.g., the stimulatory oligomeric streptavidin mutein reagent, is removed or separated from the cells or cell populations by contact or exposure to a competition reagent, e.g., biotin or a biotin analog such as D-biotin, after or during the incubation, e.g., an incubation described herein such as in Section I-D. In certain embodiments, the cells or cell population are contacted or exposed to a competition reagent, e.g., biotin or a biotin analog such as D-biotin, to remove stimulatory oligomeric reagent, e.g., the stimulatory oligomeric streptavidin mutein reagent, after the incubation but prior to steps for collecting, harvesting, or formulating the cells. In particular embodiments, the cells or cell population are contacted or exposed to a competition reagent, e.g., biotin or a biotin analog such as D-biotin, to remove the stimulatory oligomeric reagent, e.g., the stimulatory oligomeric streptavidin mutein reagent, after the incubation. In some aspects, when stimulatory oligomeric reagent, e.g., the stimulatory oligomeric streptavidin mutein reagent, is separated or removed from the cells during the incubation, e.g., by contact or exposure to a competition reagent, e.g., biotin or a biotin analog such as D-biotin, the cells are returned to the same incubation conditions as prior to the separation or removal for the remaining duration of the incubation.

In some embodiments, the cells are contacted with, with about, or with at least at or about 0.01 μM, 0.05 μM, 0.1 μM, 0.5 μM, 1 μM, 2 μM, 3 μM, 4 μM, 5 μM, 10 μM, 100 μM, 500 μM, 0.01 mM, 1 mM, or 10 mM of the competition reagent to remove or separate the oligomeric stimulatory reagent from the cells. In various embodiments, the cells are contacted with, with about, or with at least at or about 0.01 μM, 0.05 μM, 0.1 μM, 0.5 μM, 1 μM, 2 μM, 3 μM, 4 μM, 5 μM, 10 μM, 100 μM, 500 μM, 0.01 mM, 1 mM, or 10 mM of biotin or a biotin analog such as D-biotin, to remove or separate the stimulatory streptavidin mutein oligomers with reversibly attached anti-CD3 and anti-CD28 Fabs from the cells.

In particular embodiments, the stimulatory oligomeric reagent, e.g., the stimulatory oligomeric streptavidin mutein reagent, is removed or separated from the cells within or within about 120 hours, 108 hours, 96 hours, 84 hours, 72 hours, 60 hours, 48 hours, 36 hours, 24 hours, or 12 hours, inclusive, of the initiation of the stimulation. In particular embodiments, the stimulatory oligomeric reagent, e.g., the stimulatory oligomeric streptavidin mutein reagent, is removed or separated from the cells at or at about 48 hours after the stimulation is initiated. In certain embodiments, the stimulatory oligomeric reagent, e.g., the stimulatory oligomeric streptavidin mutein reagent, is removed or separated from the cells at or at about 72 hours after the stimulation is initiated. In some embodiments, the stimulatory oligomeric reagent, e.g., the stimulatory oligomeric streptavidin mutein reagent is removed or separated from the cells at or at about 96 hours after the stimulation is initiated.

C. Viral Vector Particles

In some embodiments, the viral vector particles are retroviral vector particles, such as lentiviral particles, containing a nucleic acid encoding a recombinant and/or heterologous molecule, e.g., recombinant or heterologous protein, such as a recombinant and/or heterologous receptor, such as chimeric antigen receptor (CAR) or other antigen receptor, in a genome of the viral vector. The genome of the viral vector particle typically includes sequences in addition to the nucleic acid (e.g., polynucleotide) encoding the recombinant molecule. Such sequences may include sequences that allow the genome to be packaged into the virus particle and/or sequences that promote expression of a nucleic acid encoding a recombinant receptor, such as a CAR.

1. Viral Vector

In some embodiments, the viral vector particles contain a genome derived from a retroviral genome based vector, such as derived from a lentiviral genome based vector. In some embodiments, the viral vector particle is a lentiviral vector particle. In some aspects of the provided viral vectors, a heterologous nucleic acid (e.g., polynucleotide) encoding a recombinant protein, such as an antigen receptor, such as a chimeric antigen receptor (CAR) or a transgenic T cell receptor (TCR), is contained and/or located between the 5′ LTR and 3′ LTR sequences of the vector genome. In some embodiments, the recombinant protein is an antigen receptor. In some embodiments, the recombinant protein is a T cell receptor (TCR). In some embodiments, the recombinant protein is a chimeric antigen receptor (CAR).

In some embodiments, the viral vector genome is a lentivirus genome, such as an HIV-1 genome or an SIV genome. In some embodiments, the lentiviral vector particle is replication defective. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid (e.g., polynucleotide) into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid (e.g., polynucleotide) into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid (e.g., polynucleotide) of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid (e.g., polynucleotide) used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1 and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid (e.g., polynucleotide) of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

2 Nucleic Acid Encoding a Heterologous Protein

In some embodiments, the viral vector contains a nucleic acid (e.g., polynucleotide) that encodes a heterologous recombinant protein. In some embodiments, the heterologous recombinant protein or molecule is or includes a recombinant receptor, e.g., an antigen receptor, SB-transposons, e.g., for gene silencing, capsid-enclosed transposons, homologous double stranded nucleic acid, e.g., for genomic recombination or reporter genes (e.g., fluorescent proteins, such as GFP) or luciferase).

In some embodiments, the viral vector contains a nucleic acid (e.g., polynucleotide) that encodes a recombinant receptor and/or chimeric receptor, such as a heterologous receptor protein. The recombinant receptor, such as heterologous receptor, may include antigen receptors, such as functional non-TCR antigen receptors, including chimeric antigen receptors (CARs), and other antigen-binding receptors such as transgenic T cell receptors (TCRs). The receptors may also include other receptors, such as other chimeric receptors, such as receptors that bind to particular ligands and having transmembrane and/or intracellular signaling domains similar to those present in a CAR.

In any of such examples, the nucleic acid (e.g., polynucleotide) is inserted or located in a region of the viral vector, such as generally in a non-essential region of the viral genome. In some embodiments, the nucleic acid (e.g., polynucleotide) is inserted into the viral genome in the place of certain viral sequences to produce a virus that is replication defective.

In some embodiments, the encoded recombinant antigen receptor, e.g., CAR, is one that is capable of specifically binding to one or more ligand on a cell or disease to be targeted, such as a cancer, infectious disease, inflammatory or autoimmune disease, or other disease or condition, including those described herein for targeting with the provided methods and compositions.

In certain embodiments, an exemplary antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Ra), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b, or CD30.

In some embodiments, the exemplary antigens are orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, OEPHa2, ErbB2, 3, or 4, FBP, fetal acetylcholine receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by and/or characteristic of or specific for HIV, HCV, HBV, HPV, and/or other pathogens and/or oncogenic versions thereof.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

Antigen receptors, including CARs and recombinant TCRs, and production and introduction thereof, in some embodiments include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75.

a. Chimeric Antigen Receptors

In some embodiments, the nucleic acid (e.g., polynucleotide) contained in a genome of the viral vector encodes a chimeric antigen receptor (CAR). The CAR is generally a genetically engineered receptor with an extracellular ligand binding domain, such as an extracellular portion containing an antibody or fragment thereof, linked to one or more intracellular signaling components. In some embodiments, the chimeric antigen receptor includes a transmembrane domain and/or intracellular domain linking the extracellular domain and the intracellular signaling domain. Such molecules typically mimic or approximate a signal through a natural antigen receptor and/or signal through such a receptor in combination with a costimulatory receptor.

In some embodiments, CARs are constructed with a specificity for a particular marker, such as a marker expressed in a particular cell type to be targeted by adoptive therapy, e.g., a cancer marker and/or any of the antigens described. Thus, the CAR typically includes one or more antigen-binding fragment, domain, or portion of an antibody, or one or more antibody variable domains, and/or antibody molecules. In some embodiments, the CAR includes an antigen-binding portion or portions of an antibody molecule, such as a variable heavy chain (VH) or antigen-binding portion thereof, or a single-chain antibody fragment (scFv) derived from the variable heavy (VH) and variable light (VL) chains of a monoclonal antibody (mAb).

In some embodiments, engineered cells, such as T cells, are provided that express a CAR with specificity for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In particular embodiments, the recombinant receptor, such as chimeric receptor, contains an intracellular signaling region, which includes a cytoplasmic signaling domain or region (also interchangeably called an intracellular signaling domain or region), such as a cytoplasmic (intracellular) region capable of inducing a primary activation signal in a T cell, for example, a cytoplasmic signaling domain or region of a T cell receptor (TCR) component (e.g., a cytoplasmic signaling domain or region of a zeta chain of a CD3-zeta (CD3ζ) chain or a functional variant or signaling portion thereof) and/or that comprises an immunoreceptor tyrosine-based activation motif (ITAM). In some embodiments, the CAR comprises an extracellular antigen-recognition domain that specifically binds to a target antigen and an intracellular signaling domain comprising an ITAM. In some embodiments, the intracellular signaling domain comprises an intracellular domain of a CD3-zeta (CD3ζ) chain.

In some embodiments, the chimeric receptor further contains an extracellular ligand-binding domain that specifically binds to a ligand (e.g., antigen) antigen. In some embodiments, the chimeric receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No. WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, the recombinant receptor, such as a chimeric receptor (e.g., CAR), includes a ligand-binding domain that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some embodiments, the antigen is associated with a disease or condition, such as cancer, an autoimmune disease or disorder, or an infectious disease. In some embodiments, the antigen receptor, e.g., CAR, specifically binds to a universal tag.

In some embodiments, the CAR contains an antibody or an antigen-binding fragment (e.g., scFv) that specifically recognizes an antigen, such as an intact antigen, expressed on the surface of a cell.

In some embodiments, the antigen (or a ligand) is a tumor antigen or cancer marker. In some embodiments, the antigen (or a ligand) the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen_{1B} (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Ra), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b, or CD30.

In some embodiments, the antigen or antigen binding domain is CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the scFv is derived from FMC63. FMC63 generally refers to a mouse monoclonal IgG1 antibody raised against Nalm-1 and -16 cells expressing CD19 of human origin (Ling, N. R., et al. (1987). Leucocyte typing II. 302). In some embodiments, the FMC63 antibody comprises CDRH1 set forth in SEQ ID NOS: 60, CDRH2 set forth in SEQ ID NO: 61, and CDRH3 set forth in SEQ ID NO: 62 or SEQ ID NO:76, and CDRL1 set forth in SEQ ID NO: 57 and CDR L2 set forth in SEQ ID NO: 58 or 77 and CDR L3 set forth in SEQ ID NO: 59 or 78. In some embodiments, the FMC63 antibody comprises the heavy chain variable region (VH) comprising the amino acid sequence of SEQ ID NO: 63 and the light chain variable region (V_{L}) comprising the amino acid sequence of SEQ ID NO: 64.

In some embodiments, the scFv comprises a variable light chain containing the CDRL1 sequence of SEQ ID NO:57, a CDRL2 sequence of SEQ ID NO:58, and a CDRL3 sequence of SEQ ID NO:59 and a variable heavy chain containing a CDRH1 sequence of SEQ ID NO:60, a CDRH2 sequence of SEQ ID NO:61, and a CDRH3 sequence of SEQ ID NO:62. In some embodiments, the scFv comprises a variable light chain containing the CDRL1 sequence of SEQ ID NO:57, a CDRL2 sequence of SEQ ID NO:77, and a CDRL3 sequence of SEQ ID NO:78 and a variable heavy chain containing a CDRH1 sequence of SEQ ID NO:60, a CDRH2 sequence of SEQ ID NO:61, and a CDRH3 sequence of SEQ ID NO:76.

In some embodiments, the scFv comprises a variable heavy chain region set forth in SEQ ID NO:63 and a variable light chain region set forth in SEQ ID NO:64. In some embodiments, the variable heavy and variable light chains are connected by a linker. In some embodiments, the linker is set forth in SEQ ID NO:80. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:65 or a sequence that exhibits at least 85%, 86%, 87%, 88%, 89%, 90%, 91%, 92%, 93%, 94%, 95%, 96%, 97%, 98%, or 99% sequence identity to SEQ ID NO:65. In some embodiments, the scFv comprises the sequence of amino acids set forth in SEQ ID NO:65 or a sequence that exhibits at least 85%, 86%, 87%, 88%, 89%, 90%, 91%, 92%, 93%, 94%, 95%, 96%, 97%, 98%, or 99% sequence identity to SEQ ID NO:65.

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDRH1, H2 and H3 set forth in SEQ ID NOS: 69-71, respectively, and CDRL1, L2 and L3 sequences set forth in SEQ ID NOS: 66-68, respectively. In some embodiments, the SJ25C1 antibody comprises the heavy chain variable region (V_{H}) comprising the amino acid sequence of SEQ ID NO: 72 and the light chain variable region (V_{L}) Comprising the amino acid sequence of SEQ ID NO: 73.

In some embodiments, the scFv comprises a variable light chain containing the CDRL1 sequence of SEQ ID NO:66, a CDRL2 sequence of SEQ ID NO: 67, and a CDRL3 sequence of SEQ ID NO:68 and a variable heavy chain containing a CDRH1 sequence of SEQ ID NO:69, a CDRH2 sequence of SEQ ID NO:70, and a CDRH3 sequence of SEQ ID NO:71. In some embodiments, the scFv comprises a variable heavy chain region set forth in SEQ ID NO:72 and a variable light chain region set forth in SEQ ID NO:73. In some embodiments, the variable heavy and variable light chain are connected by a linker. In some embodiments, the linker is set forth in SEQ ID NO:74. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:75 or a sequence that exhibits at least 85%, 86%, 87%, 88%, 89%, 90%, 91%, 92%, 93%, 94%, 95%, 96%, 97%, 98%, or 99% sequence identity to SEQ ID NO:75.

In some embodiments, the antibody or an antigen-binding fragment (e.g. scFv or V_{H} domain) specifically recognizes an antigen, such as BCMA. In some embodiments, the antibody or antigen-binding fragment is derived from, or is a variant of, antibodies or antigen-binding fragment that specifically binds to BCMA.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human antibodies, and cells expressing such chimeric receptors have been previously described. See Carpenter et al., Clin Cancer Res., 2013, 19(8):2048-2060, WO 2016/090320, WO2016090327, WO2010104949A2 and WO2017173256. In some embodiments, the antigen or antigen binding domain is BCMA. In some embodiments, the scFv contains a VH and a VL derived from an antibody or an antibody fragment specific to BCMA. In some embodiments, the antibody or antibody fragment that binds BCMA is or contains a VH and a VL from an antibody or antibody fragment set forth in International Patent Applications, Publication Number WO 2016/090327 and WO 2016/090320.

In some embodiments, the antigen or antigen binding domain is GPRC5D. In some embodiments, the scFv contains a VH and a VL derived from an antibody or an antibody fragment specific to GPRC5D. In some embodiments, the antibody or antibody fragment that binds GPRC5D is or contains a VH and a VL from an antibody or antibody fragment set forth in International Patent Applications, Publication Number WO 2016/090329 and WO 2016/090312.

In some aspects, the CAR contains a ligand- (e.g., antigen-) binding domain that binds or recognizes, e.g., specifically binds, a universal tag or a universal epitope. In some aspects, the binding domain can bind a molecule, a tag, a polypeptide and/or an epitope that can be linked to a different binding molecule (e.g., antibody or antigen-binding fragment) that recognizes an antigen associated with a disease or disorder. Exemplary tag or epitope includes a dye (e.g., fluorescein isothiocyanate) or a biotin. In some aspects, a binding molecule (e.g., antibody or antigen-binding fragment) linked to a tag, that recognizes the antigen associated with a disease or disorder, e.g., tumor antigen, with an engineered cell expressing a CAR specific for the tag, to effect cytotoxicity or other effector function of the engineered cell. In some aspects, the specificity of the CAR to the antigen associated with a disease or disorder is provided by the tagged binding molecule (e.g., antibody), and different tagged binding molecule can be used to target different antigens. Exemplary CARs specific for a universal tag or a universal epitope include those described, e.g., in U.S. Pat. No. 9,233,125, WO 2016/030414, Urbanska et al., (2012) Cancer Res 72: 1844-1852, and Tamada et al., (2012). Clin Cancer Res 18:6436-6445.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens. In some embodiments, the CAR contains a TCR-like antibody, such as an antibody or an antigen-binding fragment (e.g., scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e., MHC-peptide complex, for presentation of an antigen in a conformation recognizable by an antigen receptor on T cells, such as a TCRs or TCR-like antibody. Generally, MHC class I molecules are heterodimers having a membrane spanning α chain, in some cases with three α domains, and a non-covalently associated β2 microglobulin. Generally, MHC class II molecules are composed of two transmembrane glycoproteins, a and J, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g., US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen, or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g., US published application No. US20020150914, US2014/0294841; and Cohen C J. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the chimeric antigen receptor, including TCR-like CARs, includes an extracellular portion containing an antibody or antibody fragment. In some embodiments, the antibody or fragment includes an scFv. In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the extracellular portion of the CAR, such as an antibody portion thereof, further includes a spacer, such as a spacer region between the antigen-recognition component, e.g. scFv, and a transmembrane domain. The spacer may be or include at least a portion of an immunoglobulin constant region or variant or modified version thereof, such as a hinge region, e.g., an IgG4 hinge region, and/or a CH1/CL and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. In some embodiments, the spacer has the sequence set forth in SEQ ID NO: 8, and is encoded by the sequence set forth in SEQ ID NO: 9. In some embodiments, the spacer has the sequence set forth in SEQ ID NO: 10. In some embodiments, the spacer has the sequence set forth in SEQ ID NO: 11.

In some embodiments, the constant region or portion is of IgD. In some embodiments, the spacer has the sequence set forth in SEQ ID NO: 12. In some embodiments, the spacer has a sequence of amino acids that exhibits at least or at least about 85%, 86%, 87%, 88%, 89%, 90%, 91%, 92%, 93%, 94%, 95%, 96%, 97%, 98%, or 99% or more sequence identity to any of SEQ ID NOS: 8, 10, 11, and 12.

In some embodiments, the spacer may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014/031687. In some embodiments, the spacer has the sequence set forth in SEQ ID NO: 8, and is encoded by the sequence set forth in SEQ ID NO: 9. In some embodiments, the spacer has the sequence set forth in SEQ ID NO: 10. In some embodiments, the spacer has the sequence set forth in SEQ ID NO: 11.

In some embodiments, the constant region or portion is of IgD. In some embodiments, the spacer has the sequence set forth in SEQ ID NO: 12. In some embodiments, the spacer has a sequence of amino acids that exhibits at least or at least about 85%, 86%, 87%, 88%, 89%, 90%, 91%, 92%, 93%, 94%, 95%, 96%, 97%, 98%, or 99% or more sequence identity to any of SEQ ID NOS: 8, 10, 11, and 12.

The extracellular ligand binding, such as antigen recognition domain, generally is linked to one or more intracellular signaling components, such as signaling components that mimic activation through an antigen receptor complex, such as a TCR complex, in the case of a CAR, and/or signal via another cell surface receptor. In some embodiments, a transmembrane domain links the extracellular ligand binding domain and intracellular signaling domains. In some embodiments, the antigen binding component (e.g., antibody) is linked to one or more transmembrane and intracellular signaling regions. In some embodiments, the CAR includes a transmembrane domain fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, or CD154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The recombinant receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor 7 and CD8, CD4, CD25, or CD16.

In some embodiments, upon ligation of the CAR or other chimeric receptor, the cytoplasmic domain and/or region or intracellular signaling domain and/or region of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by at least two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CD8, CD22, CD79a, CD79b, and CD66d. In certain embodiments, ITAM containing primary cytoplasmic signaling sequences include those derived from TCR or CD3 zeta, FcR gamma, or FcR beta. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, CD27, DAP10, and/or ICOS. In some aspects, the same CAR includes both the activating or signaling region and costimulatory components. In some embodiments, the intracellular signaling domain comprises an intracellular signaling domain of a T cell costimulatory molecule. In some embodiments, the T cell costimulatory molecule is selected from the group consisting of CD28 and 41BB.

In some embodiments, the activating domain is included within one CAR, whereas the costimulatory component is provided by another CAR recognizing another antigen. In some embodiments, the CARs include activating or stimulatory CARs, and costimulatory CARs, both expressed on the same cell (see WO2014/055668). In some aspects, the CAR is the stimulatory or activating CAR; in other aspects, it is the costimulatory CAR. In some embodiments, the cells further include inhibitory CARs (iCARs, see Fedorov et al., Sci. Transl. Medicine, 5(215) (December, 2013), such as a CAR recognizing a different antigen, whereby an activating signal delivered through a CAR recognizing a first antigen is diminished or inhibited by binding of the inhibitory CAR to its ligand, e.g., to reduce off-target effects.

In certain embodiments, the intracellular signaling domain comprises a CD28 transmembrane and signaling domain linked to a CD3 intracellular domain. In some embodiments, the intracellular signaling domain comprises a chimeric CD28 and CD137 co-stimulatory domains, linked to a CD3 intracellular domain.

In some embodiments, the intracellular signaling domain of the CD8⁺ cytotoxic T cells is the same as the intracellular signaling domain of the CD4⁺ helper T cells. In some embodiments, the intracellular signaling domain of the CD8⁺ cytotoxic T cells is different than the intracellular signaling domain of the CD4⁺ helper T cells.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the recombinant receptor(s), e.g., CAR, encoded by nucleic acid(s) (e.g., polynucleotide(s)) within the provided viral vectors further include one or more marker, e.g., for purposes of confirming transduction or engineering of the cell to express the receptor and/or selection and/or targeting of cells expressing molecule(s) encoded by the polynucleotide. In some aspects, such a marker may be encoded by a different nucleic acid or polynucleotide, which also may be introduced during the genetic engineering process, typically via the same method, e.g., transduction by any of the methods provided herein, e.g., via the same vector or type of vector.

In some aspects, the marker, e.g., transduction marker, is a protein and/or is a cell surface molecule. Exemplary markers are truncated variants of a naturally-occurring, e.g., endogenous markers, such as naturally-occurring cell surface molecules. In some aspects, the variants have reduced immunogenicity, reduced trafficking function, and/or reduced signaling function compared to the natural or endogenous cell surface molecule. In some embodiments, the marker is a truncated version of a cell surface receptor, such as truncated EGFR (tEGFR). In some aspects, the marker includes all or part (e.g., truncated form) of CD34, an NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A P2A, E2A and/or F2A. See, e.g., WO2014/031687.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular ligand-binding portion, such as an antigen-binding portion, such as an antibody or fragment thereof and in intracellular domain. In some embodiments, the antibody or fragment includes an scFv or a single-domain VH antibody and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking and/or disposed between the extracellular domain and the intracellular signaling region or domain.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 15 or a sequence of amino acids that exhibits at least or at least about 85%, 86%, 87%, 88%, 89%, 90%, 91%, 92%, 93%, 94%, 95%, 96%, 97%, 98%, or 99% or more sequence identity to SEQ ID NO: 15; in some embodiments, the transmembrane-domain containing portion of the recombinant receptor comprises the sequence of amino acids set forth in SEQ ID NO: 16 or a sequence of amino acids having at least or at least about 85%, 86%, 87%, 88%, 89%, 90%, 91%, 92%, 93%, 94%, 95%, 96%, 97%, 98%, or 99% or more sequence identity thereto.

In some embodiments, the chimeric antigen receptor contains an intracellular domain of a T cell costimulatory molecule. In some aspects, the T cell costimulatory molecule is CD28 or 4-1BB.

In some embodiments, the intracellular domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling region and/or domain can comprise the sequence of amino acids set forth in SEQ ID NO: 17 or 18 or a sequence of amino acids that exhibits at least or at least about 85%, 86%, 87%, 88%, 89%, 90%, 91%, 92%, 93%, 94%, 95%, 96%, 97%, 98%, or 99% or more sequence identity to SEQ ID NO: 17 or 18. In some embodiments, the intracellular region and/or domain comprises an intracellular costimulatory signaling domain of 4-1BB or functional variant thereof, such as a 42-amino acid cytoplasmic domain of a human 4-1BB (Accession No. Q07011.1), or functional variant or portion thereof, such as the sequence of amino acids set forth in SEQ ID NO: 19 or a sequence of amino acids that exhibits at least or at least about 85%, 86%, 87%, 88%, 89%, 90%, 91%, 92%, 93%, 94%, 95%, 96%, 97%, 98%, or 99% or more sequence identity to SEQ ID NO: 19.

In some embodiments, the intracellular signaling region and/or domain comprises a human CD3 chain, optionally a CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190 or 8,911,993. In some embodiments, the intracellular signaling region comprises the sequence of amino acids set forth in SEQ ID NO: 20, 21, or 22 or a sequence of amino acids that exhibits at least or at least about 85%, 86%, 87%, 88%, 89%, 90%, 91%, 92%, 93%, 94%, 95%, 96%, 97%, 98%, or 99% or more sequence identity to SEQ ID NO: 20, 21, or 22.

In some aspects, the spacer contains only a hinge region of an IgG, such as only a hinge of IgG4 or IgG1 such as the hinge only spacer set forth in SEQ ID NO: 8. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to C_{H}2 and C_{H}3 domains, such as set forth in SEQ ID NO: 10. In some embodiments, the spacer is an Ig hinge, e.g., an IgG4 hinge, linked to a C_{H}3 domain only, such as set forth in SEQ ID NO: 11. In some embodiments, the spacer is or comprises a glycine-serine rich sequence or other flexible linker such as known flexible linkers.

For example, in some embodiments, the CAR includes: an extracellular ligand-binding portion, such as an antigen-binding portion, such as an antibody or fragment thereof, including sdAbs and scFvs, that specifically binds an antigen, e.g., an antigen described herein; a spacer such as any of the Ig-hinge containing spacers; a transmembrane domain that is a portion of CD28 or a variant thereof; an intracellular signaling domain containing a signaling portion of CD28 or functional variant thereof; and a signaling portion of CD3 zeta signaling domain or functional variant thereof. In some embodiments, the CAR includes: an extracellular ligand-binding portion, such as an antigen-binding portion, such as an antibody or fragment thereof, including sdAbs and scFvs, that specifically binds an antigen, e.g., an antigen described herein; a spacer such as any of the Ig-hinge containing spacers; a transmembrane domain that is a portion of CD28 or a variant thereof; an intracellular signaling domain containing a signaling portion of 4-1BB or functional variant thereof; and a signaling portion of CD3 zeta signaling domain or functional variant thereof.

In some embodiments, such CAR constructs further includes a T2A ribosomal skip element and/or a tEGFR sequence, e.g., downstream of the CAR. In some embodiments, nucleic acid molecules encoding such CAR constructs further includes a sequence encoding a ribosomal skip element (e.g. T2A) followed by a sequence encoding a a tEGFR sequence, e.g., downstream of the sequence encoding the CAR. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A), equine rhinitis A virus (E2A), Thosea asigna virus (T2A), and porcine teschovirus-1 (P2A) as described in U.S. Patent Publication No. 20070116690.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

b. T Cell Receptors (TCRs)

In some embodiments, the recombinant molecule(s) encoded by the nucleic acid(s) (e.g., polynucleotide) is or include a recombinant T cell receptor (TCR). In some embodiments, the recombinant TCR is specific for an antigen, generally an antigen present on a target cell, such as a tumor-specific antigen, an antigen expressed on a particular cell type associated with an autoimmune or inflammatory disease, or an antigen derived from a viral pathogen or a bacterial pathogen. In some embodiments, engineered cells, such as T cells, are provided that express a TCR or antigen-binding portion thereof that recognizes an peptide epitope or T cell epitope of a target polypeptide, such as an antigen of a tumor, viral, or autoimmune protein. In some embodiments, the TCR specifically binds to an antigen associated with a disease or condition or specifically binds to a universal tag. In some embodiments, the antigen is associated with a disease or condition, such as cancer, an autoimmune disease or disorder, or an infectious disease.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the 3-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g., CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids (e.g., polynucleotides) encoding the TCR can be obtained from a variety of sources, such as by polymerase chain reaction (PCR) amplification of TCR-encoding nucleic acids (e.g., polynucleotides) within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e., normal TCR libraries. In some embodiments, the TCRs can be amplified from a T cell source of a diseased subject, i.e., diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g., present on the antigen-specific T cells, may be selected, such as by binding activity, e.g., particular affinity or avidity for the antigen.

In some embodiments, the genetically engineered antigen receptors include recombinant T cell receptors (TCRs) and/or TCRs cloned from naturally occurring T cells. In some embodiments, the TCR is one that has been cloned from naturally occurring T cells. In some embodiments, a high-affinity T cell clone for a target antigen (e.g., a cancer antigen) is identified and isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354. In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent, or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified by a skilled artisan. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, and WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known. See e.g., Soo Hoo, W. F. et al. PNAS (USA) 89, 4759 (1992); Wulfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g., International published PCT No. WO 03/020763). In some embodiments, a scTCR is a non-disulfide linked truncated TCR in which heterologous leucine zippers fused to the C-termini thereof facilitate chain association (see e.g., International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P- wherein P is proline, G is glycine and S is serine (SEQ ID NO: 29). In some embodiments, the linker has the sequence

(SEQ ID NO: 30)
GSADDAKKDAAKKDGKS

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, nucleic acid or nucleic acids (e.g., polynucleotide(s)) encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can be a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBIl21, and pBIN19 (Clontech). In some embodiments, animal expression vectors include pEUK-Cl, pMAM, and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, or a promoter found in the long-terminal repeat of the murine stem cell virus. Other known promoters also are contemplated.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are coexpressed. In some embodiments, the nucleic acid (e.g., polynucleotide) encoding a TCR further includes a marker to confirm transduction or engineering of the cell to express the receptor. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g., lentiviral, vector.

c. Chimeric Auto-Antibody Receptor (CAAR)

In some embodiments, the recombinant receptor is a chimeric autoantibody receptor (CAAR). In some embodiments, the CAAR is specific for an autoantibody. In some embodiments, a cell expressing the CAAR, such as a T cell engineered to express a CAAR, can be used to specifically bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent application Pub. No. US 2017/0051035.

In some embodiments, the CAAR comprises an autoantibody binding domain, a transmembrane domain, and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM). In some embodiments, the intracellular signaling region comprises a secondary or costimulatory signaling region (secondary intracellular signaling regions).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

d. Multi-Targeting

In some embodiments, the cells and methods include multi-targeting strategies, such as expression of two or more genetically engineered receptors on the cell, each recognizing the same of a different antigen and typically each including a different intracellular signaling component. Such multi-targeting strategies are described, for example, in International Patent Application Publication No: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR or TCR) which is capable of inducing an activating or stimulating signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR or TCR) is capable of inducing an activating or stimulating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF-κB and/or AP-1, and/or induction of gene expression of factors such as cytokines, proliferation, and/or survival.

In some embodiments, the first and/or second receptor includes intracellular signaling domains of costimulatory receptors such as CD28, CD137 (4-1BB), OX40, and/or ICOS. In some embodiments, the first and second receptor include an intracellular signaling domain of a costimulatory receptor that are different. In one embodiment, the first receptor contains a CD28 costimulatory signaling region and the second receptor contain a 4-1BB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating or stimulating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

e. Other Regulatory Elements

In some embodiments of the methods and compositions provided herein, the nucleic acid (e.g., polynucleotide) sequence contained in the viral vector genome encoding an recombinant receptor, such as an antigen receptor, for example a CAR, is operably linked in a functional relationship with other genetic elements, for example transcription regulatory sequences including promoters or enhancers, to regulate expression of the sequence of interest in a particular manner. In certain instances, such transcriptional regulatory sequences are those that are temporally and/or spatially regulated with respect to activity. Expression control elements that can be used for regulating the expression of the components are known and include, but are not limited to, inducible promoters, constitutive promoters, secretion signals, enhancers and other regulatory elements. In some embodiments, the nucleic acid (e.g., polynucleotide) sequence contained in the viral vector genome contain multiple expression control elements that control different encoded components, e.g., different receptor components and/or signaling components, such that the expression, function and/or activity of the recombinant receptor and/or the engineered cell, e.g. cell expressing the engineered receptor, can be regulated, e.g., are inducible, repressible, regulatable and/or user controlled. In some embodiments, one or more vectors can contain one or more nucleic acid (e.g., polynucleotide) sequences that contain one or more expression control elements and/or one or more encoded components, such that the nucleic acid sequences together can regulate the expression, activity and/or function of the encoded components, e.g., recombinant receptor, or the engineered cell.

In some embodiments, the nucleic acid (e.g., polynucleotide) sequence encoding a recombinant receptor, such as an antigen receptor, for example a CAR, is operably linked with internal promoter/enhancer regulatory sequences. The promoters employed may be constitutive, tissue-specific, inducible, and/or useful under the appropriate conditions to direct high level expression of the introduced DNA segment. The promoter may be heterologous or endogenous. In some embodiments, a promoter and/or enhancer is produced synthetically. In some embodiments, a promoter and/or enhancer is produced using recombinant cloning and/or nucleic acid amplification technology.

In some cases, the nucleic acid (e.g., polynucleotide) sequence encoding the recombinant receptor contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO: 32 and encoded by the nucleotide sequence set forth in SEQ ID NO: 31. In some cases, the nucleic acid (e.g., polynucleotide) sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 32 and encoded by the nucleotide sequence set forth in SEQ ID NO: 31, or the CD8 alpha signal peptide set forth in SEQ ID NO: 33.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor.

In certain cases where nucleic acid molecules encode two or more different polypeptide chains, e.g., a recombinant receptor and a marker, each of the polypeptide chains can be encoded by a separate nucleic acid molecule. For example, two separate nucleic acids are provided, and each can be individually transferred or introduced into the cell for expression in the cell. In some embodiments, the nucleic acid encoding the recombinant receptor and the nucleic acid encoding the marker are operably linked to the same promoter and are optionally separated by an internal ribosome entry site (IRES), or a nucleic acid encoding a self-cleaving peptide or a peptide that causes ribosome skipping, which optionally is a T2A, a P2A, a E2A or a F2A. In some embodiments, the nucleic acids encoding the marker and the nucleic acid encoding the recombinant receptor are operably linked to two different promoters. In some embodiments, the nucleic acid encoding the marker and the nucleic acid encoding the recombinant receptor are present or inserted at different locations within the genome of the cell. In some embodiments, the polynucleotide encoding the recombinant receptor is introduced into a composition containing cultured cells, such as by retroviral transduction, transfection, or transformation.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products (e.g., encoding the marker and encoding the recombinant receptor) by a message from a single promoter. Alternatively, in some cases, a single promoter may direct expression of an RNA that contains, in a single open reading frame (ORF), two or three genes (e.g., encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 28), equine rhinitis A virus (E2A, e.g., SEQ ID NO: 27), Thosea asigna virus (T2A, e.g., SEQ ID NO: 13 or 24), and porcine teschovirus-1 (P2A, e.g., SEQ ID NO: 25 or 26) as described in U.S. Patent Publication No. 20070116690.

Any of the recombinant receptors described herein can be encoded by polynucleotides containing one or more nucleic acid sequences encoding recombinant receptors, in any combinations or arrangements. For example, one, two, three or more polynucleotides can encode one, two, three or more different polypeptides, e.g., recombinant receptors. In some embodiments, one vector or construct contains a nucleic acid sequence encoding marker, and a separate vector or construct contains a nucleic acid sequence encoding a recombinant receptor, e.g., CAR. In some embodiments, the nucleic acid encoding the marker and the nucleic acid encoding the recombinant receptor are operably linked to two different promoters. In some embodiments, the nucleic acid encoding the recombinant receptor is present downstream of the nucleic acid encoding the marker.

In some embodiments, the vector backbone contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g., CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, a E2A, or a F2A. Extrinsic marker genes may in some cases be utilized in connection with engineered cell to permit detection or selection of cells and, in some cases, also to promote cell suicide.

Exemplary surrogate markers can include truncated cell surface polypeptides, such as a truncated human epidermal growth factor receptor 2 (tHER2), a truncated epidermal growth factor receptor (EGFRt, exemplary EGFRt sequence set forth in SEQ ID NO: 14 or 23) or a prostate-specific membrane antigen (PSMA) or modified form thereof. EGFRt may contain an epitope recognized by the antibody cetuximab (Erbitux®) or other therapeutic anti-EGFR antibody or binding molecule, which can be used to identify or select cells that have been engineered with the EGFRt construct and a recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434). In some aspects, the marker, e.g., surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g., tEGFR) comprises the sequence of amino acids set forth in SEQ ID NO: 14 or 23 or a sequence of amino acids that exhibits at least 85%, 86%, 87%, 88%, 89%, 90%, 91%, 92%, 93%, 94%, 95%, 96%, 97%, 98%, or 99% or more sequence identity to SEQ ID NO: 14 or 23.

In some embodiments, the marker is or comprises a fluorescent protein, such as green fluorescent protein (GFP), enhanced green fluorescent protein (EGFP), such as super-fold GFP, red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and/or yellow fluorescent protein (YFP), and/or variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), and/or chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene, or a Zeocin resistance gene, or a modified form thereof.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments a promoter and/or enhancer may be one that is naturally associated with a nucleic acid sequence, as may be obtained by isolating the 5′ non-coding sequences located upstream of the coding segment and/or exon. Alternatively, in some embodiments the coding nucleic acid segment may be positioned under the control of a recombinant and/or heterologous promoter and/or enhancer, which is not normally associated with the coding nucleic acid sequence in the natural setting. For example, exemplary promoters used in recombinant DNA construction include, but are not limited to, the β-lactamase (penicillinase), lactose, tryptophan (trp), RNA polymerase (pol) III promoters including, the human and murine U6 pol III promoters as well as the human and murine H1 RNA pol III promoters; RNA polymerase (pol) II promoters; cytomegalovirus immediate early promoter (pCMV), elongation factor-1 alpha (EF-1 alpha), and the Rous Sarcoma virus long terminal repeat promoter (pRSV) promoter systems. In some embodiments, the promoter may be obtained, for example, from the genomes of viruses such as polyoma virus, fowlpox virus, adenovirus, bovine papilloma virus, avian sarcoma virus, cytomegalovirus, a retrovirus, hepatitis-B virus, and/or Simian Virus 40 (SV40). The promoter may also be, for example, a heterologous mammalian promoter, e.g., the actin promoter or an immunoglobulin promoter, a heat-shock promoter, or the promoter normally associated with the native sequence, provided such promoters are compatible with the target cell. In one embodiment, the promoter is the naturally occurring viral promoter in a viral expression system.

In some embodiments, the promoter may be constitutively active. Non-limiting examples of constitutive promoters that may be used include the promoter for ubiquitin (U.S. Pat. No. 5,510,474; WO 98/32869), CMV (Thomsen et al., PNAS 81:659, 1984; U.S. Pat. No. 5,168,062), beta-actin (Gunning et al. 1989 Proc. Natl. Acad. Sci. USA 84:4831-4835) and pgk (see, for example, Adra et al. 1987 Gene 60:65-74; Singer-Sam et al. 1984 Gene 32:409-417; and Dobson et al. 1982 Nucleic Acids Res. 10:2635-2637).

In some embodiments, the promoter may be a tissue specific promoter and/or a target cell-specific promoter. In some embodiments, the promoters may be selected to allow for inducible expression of the sequence of interest. A number of systems for inducible expression are known, including the tetracycline responsive system, the lac operator-repressor system, as well as promoters responsive to a variety of environmental or physiological changes, including heat shock, metal ions, such as metallothionein promoter, interferons, hypoxia, steroids, such as progesterone or glucocorticoid receptor promoter, radiation, such as VEGF promoter. In some embodiments, the tetracycline-(tet)-regulatable system, which is based on the inhibitory action of the tet repression (tetr) of Escherichia coli on the tet operator sequence (TECO), can be modified for use in mammalian systems and used as a regulatable element for expression cassettes. These systems are well known. (See, Goshen and Badgered, Proc. Natl. Acad. Sci. USA 89: 5547-51 (1992), Shockett et al., Proc. Natl. Acad. Sci. USA 92:6522-26 (1996), Lindemann et al., Mol. Med. 3:466-76 (1997)).

A combination of promoters may also be used to obtain the desired expression of the gene of interest. The artisan of ordinary skill will be able to select a promoter based on the desired expression pattern of the gene in the organism or the target cell of interest.

In some embodiments, an enhancer may also be present in the viral construct to increase expression of the gene of interest. Enhancers are typically cis-acting nucleic acid elements, usually about 10 to 300 by in length, that act on a promoter to increase its transcription. Many enhancers in viral genomes, such as HIV or CMV are known. For example, the CMV enhancer (Boshart et al. Cell, 41:521, 1985) can be used. Other examples include, for example, the SV40 enhancer on the late side of the replication origin (bp 100-270), the cytomegalovirus early promoter enhancer, the polyoma enhancer on the late side of the replication origin, and adenovirus enhancers. In some cases, an enhancer is from a mammalian gene, such as an enhancer from a globin, elastase, albumin, alpha-fetoprotein, or insulin. An enhancer can be used in combination with a heterologous promoter. The enhancer may be spliced into the vector at a position 5′ or 3′ to the polynucleotide sequence encoding the gene of interest, but is generally located at a site 5′ from the promoter. One of ordinary skill in the art will be able to select the appropriate enhancer based on the desired expression pattern.

The viral vector genome may also contain additional genetic elements. The types of elements that can be included in the constructs are not limited in any way and can be chosen by one with skill in the art.

For example, a signal that facilitates nuclear entry of the viral genome in the target cell may be included. An example of such a signal is the HIV-1 flap signal (in some cases referred to as the flap sequence). In addition, the vector genome may contain one or more genetic elements designed to enhance expression of the gene of interest. In some embodiments, the genome contains a post-transcriptional regulatory element (PRE) or modified form thereof that exhibits post-transcriptional activity. For example, in some embodiments, a woodchuck hepatitis virus posts-transcriptional responsive element (WPRE) may be placed into the construct (Zufferey et al. 1999. J. Virol. 74:3668-3681; Deglon et al. 2000. Hum. Gene Ther. 11:179-190). In some embodiments, the vector genome lacks a flap sequence and/or lacks a WPRE. In some embodiments, the vector genome contains a mutated or defective flap sequence and/or WPRE.

In some instances, more than one open reading frame encoding separate heterologous proteins can be included. For example, in some embodiments, if a reporter and/or detectable and/or selectable gene is included in the expression construct, an internal ribosomal entry site (IRES) sequence can be included. Typically, the additional genetic elements are operably linked with and controlled by an independent promoter/enhancer. The additional genetic element can be a reporter gene, a selectable marker, or other desired gene.

In some embodiments, other various regulatory elements can include a transcription initiation region and/or a termination region. Expression vectors may also contain sequences for the termination of transcription and for stabilizing the mRNA. Such sequences are known and are often found naturally in the 5′ and, occasionally 3′, untranslated regions of eukaryotic or viral DNAs or cDNAs. Examples of transcription termination region include, but are not limited to, polyadenylation signal sequences. Examples of polyadenylation signal sequences include, but are not limited to, Bovine growth hormone (BGH) poly(A), SV40 late poly(A), rabbit beta-globin (RBG) poly(A), thymidine kinase (TK) poly(A) sequences, and any variants thereof.

In some embodiments, regulatory elements can include regulatory elements and/or systems that allow regulatable expression and/or activity of the recombinant receptor, e.g., CAR. In some embodiments, regulatable expression and/or activity is achieved by configuring the recombinant receptor to contain or be controlled by particular regulatory elements and/or systems. In some embodiments, one or more additional receptors can be used in an expression regulation systems. In some embodiments, expression regulation systems can include systems that require exposure to or binding of a specific ligand that can regulate the expression and/or activity of the recombinant receptor. In some embodiments, regulated expression of the recombinant receptor, e.g., CAR, is achieved a regulatable transcription factor release system, e.g., a modified Notch signaling system (see, e.g., Roybal et al., Cell (2016) 164:770-779; Morsut et al., Cell (2016) 164:780-791). In some embodiments, regulation of activity of the recombinant receptor is achieved by administration of an additional agent that can induce conformational changes and/or multimerization of polypeptides, e.g., the recombinant receptor. In some embodiments, the additional agent is a chemical inducer (see, e.g., U.S. Patent Publication No. 2016/0046700, Clackson et al. (1998) Proc Natl Acad Sci USA. 95(18):10437-42; Spencer et al. (1993) Science 262(5136):1019-24; Farrar et al. (1996) Nature 383 (6596):178-81; Miyamoto et al. (2012) Nature Chemical Biology 8(5): 465-70; Erhart et al. (2013) Chemistry and Biology 20(4): 549-57).

3. Preparation of Viral Vector Particles

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e., a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. In some embodiments, the viral vector particle, e.g., the lentiviral vector particle, is pseudotyped with a viral envelope glycoprotein. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10), and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g. antigen receptor, such as CAR, can be detected.

D. Transduction

In any of the provided embodiments, the provided methods involve methods of transducing cells by contacting, e.g., incubating, a viral vector particle with a cell composition comprising a plurality of cells. In some embodiments, the input composition is or comprises primary cells obtained from a subject, such as cells enriched and/or selected from a subject, and/or cells that were incubated under stimulatory conditions. In some embodiments, the cell composition comprising a plurality of cells is or comprises cells that were selected and/or enriched for positive surface expression of a marker, such as CCR7. In some embodiments, the cell composition comprising a plurality of cells is or comprises cells that were selected and/or enriched for positive surface expression of CCR7, and were incubated under stimulatory conditions (hereinafter also referred to as a “stimulated composition”). In some embodiments, the cell composition is or comprises the stimulated composition.

In some embodiments, the cell composition comprises primary cells obtained from a subject. In some aspects, the sample is a whole blood sample, a buffy coat sample, a peripheral blood mononuclear cells (PBMC) sample, an unfractionated T cell sample, a lymphocyte sample, a white blood cell sample, an apheresis product, or a leukapheresis product. In some embodiments, prior to the selection, stimulation, and/or transduction of cells, the sample containing primary cells is contacted ex vivo with or contains serum or plasma at a concentration of at least or at least about 10% (v/v), at least or at least about 15% (v/v), at least or at least about 20% (v/v), at least or at least about 25% (v/v), at least or at least about 30% (v/v), at least or at least about 35% (v/v), at least or at least about 40% (v/v), or at least or at least about 50%. In some embodiments, the sample contains serum or plasma at a concentration that is or is approximately about or at least about 25%, 26%, 27%, 28%, 29%, 30%, 31%, 32%, 33%, 34%, or 35% (v/v). In some embodiments, the serum or plasma is human. In some embodiments, the serum or plasma is autologous to the subject. In some embodiments, prior to the selection, stimulation, and/or transduction of cells, the sample containing primary cells is contacted with or contains an anticoagulant. In some embodiments, the anti-coagulant is or contains free citrate ion, e.g., anticoagulant citrate dextrose solution, Solution A (ACD-A). In some embodiments, prior to the selection, stimulation, and/or transduction of cells, the sample is maintained at a temperature of from or from about 2° C. to 8° C. for up to 48 hours, such as for up to 12 hours, 24 hours, or 36 hours.

In some embodiments, the cell composition comprises and/or is enriched for CCR7+ cells, such as CCR7+ T cells. In some embodiments, the cell composition comprises and/or is enriched for T cells, including CD4+ and/or CD8+ T cells. In some embodiments, the cell composition comprises and/or is enriched for CCR7+CD4+ T cells, CCR7+CD8+ T cells, CCR7+CD3+ T cells, or CCR7+CD4+CD8+ T cells. In some aspects, enrichment can be carried out by affinity-based selection by incubation of primary cells with one or more selection or affinity reagent that specifically binds to a cell surface molecule expressed on a subpopulation of the primary cells, thereby enriching the primary cells based on binding to the selection reagent. In some embodiments, enrichment can be carried out by incubation of cells with antibody-coated particles, e.g., magnetic beads.

In some embodiments, the cell composition comprises greater than or greater than about 75%, 80%, 85%, 90%, or 95% or more T cells obtained from a sample from a subject. In some embodiments, the cell composition was not incubated under stimulatory conditions prior to transducing the cells by incubating them with a viral vector particle. In some aspects, prior to the incubation, no more than 5%, 10%, 20%, 30%, or 40% of the T cells of the cell composition are activated cells, e.g., express a surface marker selected from the group consisting of HLA-DR, CD25, CD69, CD71, CD40L, and 4-1BB; comprise intracellular expression of a cytokine selected from the group consisting of IL-2, IFN-gamma, and TNF-alpha, are in the G1 or later phase of the cell cycle, and/or are capable of proliferating. In some embodiments, a cell composition containing such cells, e.g., cells that have not been subjected to ex vivo stimulation with a stimulating agent or agents prior to the incubating and/or contacting, is one in which greater than greater than 20%, 30%, 40%, 50%, 60%, or 70% or more of the cells express the low-density lipid receptor (LDL-R). In some embodiments, the cell composition is enriched and/or selected for T cells, such as CCR7+ T cells that are also CD4+ and/or CD8+, and, prior to said incubating, greater than 20%, 30%, 40%, 50%, 60%, or 70% or more of the T cells express the low-density lipid receptor (LDL-R).

In some embodiments, the cell composition (e.g., the stimulated composition) was incubated under stimulatory conditions prior to transducing the cells by incubating them with a viral vector particle. In some aspects, prior to the incubation, at least 60%, 65%, 70%, 75%, 80%, 85%, 90%, or 95% of the T cells of the cell composition are activated cells, e.g., express a surface marker selected from the group consisting of HLA-DR, CD25, CD69, CD71, CD40L, and 4-1BB; comprise intracellular expression of a cytokine selected from the group consisting of IL-2, IFN-gamma, and TNF-alpha, are in the G1 or later phase of the cell cycle, and/or are capable of proliferating. In some aspects, prior to the incubation, at least 10%, at least 20%, at least 30%, at least 40%, at least 50%, or at least 60% of the T cells of the cell composition are activated cells, e.g., express a surface marker selected from the group consisting of HLA-DR, CD25, CD69, CD71, CD40L, and 4-1BB; comprise intracellular expression of a cytokine selected from the group consisting of IL-2, IFN-gamma, and TNF-alpha, are in the G1 or later phase of the cell cycle, and/or are capable of proliferating.

In some embodiments, during or during at least a portion of the incubating and/or contacting, the cell composition can comprise one or more cytokines. In some embodiments, the cytokine is selected from IL-2, IL-7, or IL-15. In some embodiments, the cytokine is a recombinant cytokine. In some embodiments, the concentration of the cytokine in the cell composition, independently, is from or from about 1 IU/mL to 1500 IU/mL, such as from or from about 1 IU/mL to 100 IU/mL, 2 IU/mL to 50 IU/mL, 5 IU/mL to 10 IU/mL, 10 IU/mL to 500 IU/mL, 50 IU/mL to 250 IU/mL, 100 IU/mL to 200 IU/mL, 50 IU/mL to 1500 IU/mL, 100 IU/mL to 1000 IU/mL, or 200 IU/mL to 600 IU/mL. In some embodiments, the concentration of the cytokine in the cell composition, independently, is at least or at least about 1 IU/mL, 5 IU/mL, 10 IU/mL, 50 IU/mL, 100 IU/mL, 200 IU/mL, 500 IU/mL, 1000 IU/mL, or 1500 IU/mL. In certain aspects, an agent capable of activating an intracellular signaling domain of a TCR complex, such as an anti-CD3 and/or anti-CD28 antibody, also can be including during or during at least a portion of the incubating or subsequent to the incubating.

In some embodiments, during or during at least a portion of the incubating and/or contacting, the cell composition can comprises serum. In some embodiments, the serum is fetal bovine serum. In some embodiments, the serum is human serum. In some embodiments, the serum is present in the cell composition at a concentration from or from about 0.5% to 25% (v/v), 1.0% to 10% (v/v) or 2.5% to 5.0% (v/v), each inclusive. In some embodiments, the serum is present in the cell composition at a concentration that is at least or at least about 0.5% (v/v), 1.0% (v/v), 2.5% (v/v), 5% (v/v) or 10% (v/v).

In some embodiments, during or during at least a portion of the incubating and/or contacting, the cell composition is free and/or substantially free of serum. In some embodiments, during or during at least a portion of the incubating and/or contacting, the cell composition is incubated and/or contacted in the absence of serum. In particular embodiments, during or during at least a portion of the incubating and/or contacting, the cell composition is incubated and/or contacted in serum-free media. In some embodiments, the serum free media is a defined and/or well-defined cell culture media. In some embodiments, the serum free media is formulated to support growth, proliferation, health, homeostasis of cells of a certain cell type, such as immune cells, T cells, and/or CD4+ and CD8+ T cells.

In some embodiments, during or during at least a portion of the incubating and/or contacting, the cell composition comprises N-Acetylcysteine. In some embodiments, the concentration of N-Acetylcysteine in the cell composition is from or from about 0.4 mg/mL to 4 mg/mL, 0.8 mg/mL to 3.6 mg/mL, or 1.6 mg/mL to 2.4 mg/mL, each inclusive. In some embodiments, the concentration of N-Acetylcysteine in the cell composition is at least or at least about or about 0.4 mg/mL, 0.8 mg/mL, 1.2 mg/mL, 1.6 mg/mL, 2.0 mg/mL, 2.4 mg/mL, 2.8 mg/mL, 3.2 mg/mL, 3.6 mg/mL, or 4.0 mg/mL.

In some embodiments, the concentration of cells of the cell composition is from or from about 1.0×10⁵ cells/mL to 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL, or 1×10⁸ cells/mL.

In some embodiments, the cell composition (e.g., the stimulated composition) comprises at least at or about at least or about 25×10⁶ cells, 50×10⁶ cells, 75×10⁶ cells 100×10⁶ cells, 125×10⁶ cells, 150×10⁶ cells, 175×10⁶ cells, 200×10⁶ cells, 225×10⁶ cells, 250×10⁶ cells, 275×10⁶ cells, or 300×10⁶ cells. For example, in some embodiments, the cell composition (e.g., the stimulated composition) comprises at least at or about at least or about 50×10⁶ cells, 100×10⁶ cells, or 200×10⁶ cells.

In some embodiments, the viral vector particles are provided at a certain ratio of copies of the viral vector particles or infectious units (IU) thereof, per total number of cells (IU/cell) in the cell composition or total number of cells to be transduced. For example, in some embodiments, the viral vector particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10⁶ IU/mL and 5×10⁷ IU/mL, such as at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL.

In some embodiments, transduction can be achieved at a multiplicity of infection (MOI) of less than 100, such as generally less than 60, 50, 40, 30, 20, 10, or 5 or less. In some embodiments, the viral vector particle is incubated at a multiplicity of infection of less than about 20.0 or less than or less than about 10.0. In some embodiments, the viral vector particle is incubated at a multiplicity of infection from or from about 1.0 IU/cell to 10 IU/cell; or the viral vector particle is incubated at a multiplicity of infection of at least or at least about 1.6 IU/cell, 1.8 IU/cell, 2.0 IU/cell, 2.4 IU/cell, 2.8 IU/cell, 3.2 IU/cell, 3.6 IU/cell, 4.0 IU/cell, 5.0 IU/cell, 6.0 IU/cell, 7.0 IU/cell, 8.0 IU/cell, 9.0 IU/cell, or 10.0 IU/cell.

In some embodiments, the method involves contacting or incubating, such as admixing, the cells with the viral vector particles. In some embodiments, the contacting or incubating is for 30 minutes to 72 hours, such as 30 minute to 48 hours, 30 minutes to 24 hours, or 1 hour to 24 hours, such as at least or about at least 30 minutes, 1 hour, 2 hours, 6 hours, 12 hours, 24 hours, or 36 hours or more.

In some embodiments, contacting or incubating is performed in solution. In some embodiments, the cells and viral particles are contacted in a volume of from or from about 0.5 mL to 500 mL, such as from or from about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL, or 200 mL to 500 mL.

In some embodiments, when the contacting or incubating can be effected with centrifugation, such as spinoculation (e.g., centrifugal inoculation). In some embodiments, the incubating the viral vector particle comprises a step of spinoculating the viral vector particles with the composition (e.g., the stimulated composition). In some embodiments, the composition containing cells, viral vector particles and reagent can be rotated, generally at relatively low force or speed, such as speed lower than that used to pellet the cells, such as from or from about 600 rpm to 1700 rpm (e.g., at or about or at least 600 rpm, 1000 rpm, 1500 rpm, or 1700 rpm). In some embodiments, the rotation is carried at a force, e.g., a relative centrifugal force, of from or from about 100 g to 3200 g (e.g., at or about or at least at or about 100 g, 200 g, 300 g, 400 g, 500 g, 1000 g, 1500 g, 2000 g, 2500 g, 3000 g, or 3200 g), as measured for example at an internal or external wall of the chamber or cavity. In some embodiments comprising a step of spinoculating the viral vector particle with the stimulated composition, the spinoculating step comprises rotating, in an internal cavity of a centrifugal chamber, the viral vector particles and the stimulated composition, wherein the rotation is at a relative centrifugal force at an internal surface of the side wall of the cavity that is: (a) between or between about 500 g and 2500 g, 500 g and 2000 g, 500 g and 1600 g, 500 g an 1000 g, 600 g and 1600 g, 600 g and 1000 g, 1000 g and 2000 g, or 1000 g and 1600 g, each inclusive; or (b) at least or at least about 600 g, 800 g, 1000 g, 1200 g, 1600 g, or 2000 g. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In certain embodiments, at least a portion of the engineering, transduction, and/or transfection is performed with rotation, e.g., spinoculation and/or centrifugation. In some embodiments, the rotation is performed for, for about, or for at least or about 5 minutes, 10 minutes, 15 minutes, 30 minutes, 60 minutes, 90 minutes, 1 hour, 2 hours, 3 hours, 4 hours, 6 hours, 8 hours, 12 hours, 24 hours, 48 hours, 72 hours, 2 days, 3 days, 4 days, 5 days, 6 days, or for at least 7 days. In some embodiments, the rotation is performed for or for about 60 minutes. In some embodiments, spinoculating is for a time that is: (a) greater than or about 5 minutes, greater than or about 10 minutes, greater than or about 15 minutes, greater than or about 20 minutes, greater than or about 30 minutes, greater than or about 45 minutes, greater than or about 60 minutes, greater than or about 90 minutes, or greater than or about 120 minutes; or (b) between or between about 5 minutes and 60 minutes, 10 minutes and 60 minutes, 15 minutes and 60 minutes, 15 minutes and 45 minutes, 30 minutes and 60 minutes, or 45 minutes and 60 minutes, each inclusive. In certain embodiments, the rotation is performed for about 30 minutes. In some embodiments, the rotation performed for about 30 minutes at between 600 g and 700 g, e.g., at or about 693 g.

In certain embodiments, at least a portion of the engineering, transduction, and/or transfection is conducted at a volume (e.g., the spinoculation volume) from about 5 mL to about 100 mL, such as from about 10 mL to about 50 mL, from about 15 mL to about 45 mL, from about 20 mL to about 40 mL, from about 25 mL to about 35 mL, or at or at about 30 mL. In certain embodiments, the cell pellet volume after spinoculation ranges from about 1 mL to about 25 mL, such as from about 5 mL to about 20 mL, from about 5 mL to about 15 mL, from about 5 mL to about 10 mL, or at or at about 10 mL.

In some embodiments, the incubation of the cells with the viral vector particles is carried out by contacting one or more cells of a composition with a nucleic acid molecule encoding the recombinant protein, e.g., recombinant receptor. In some embodiments, the contacting can be effected with centrifugation, such as spinoculation (e.g., centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. patent application, Publication No. US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the incubation of the cells with the viral vector particles further comprises contacting the composition (e.g., stimulated composition) and/or viral vector particles with a transduction adjuvant. In some embodiments, the contacting the composition (e.g., stimulated composition) and/or the viral vector particles with a transduction adjuvant is carried out prior to, concomitant with, or after spinoculating the viral vector particles with the composition (e.g., stimulated composition).

In some embodiments, at least a portion of the incubation of the viral vector particle is carried out at or about 37° C.±2° C. For instance, in some embodiments, at least a portion of the incubation of the viral particle is carried out at or about 35-39° C. In some embodiments, the at least a portion of the incubation of the viral vector particle that is carried out at or about 37° C.±2° C. is carried out for no more than or no more than about 2 hours, 4 hours, 12 hours, 18 hours, 24 hours, 30 hours, 36 hours, 48 hours, 60 hours, or 72 hours. In some embodiments, the at least a portion of the incubation of the viral vector particle that is carried out at or about 37° C.±2° C. is carried out for or for about 24 hours.

In some embodiments, at least a portion of the incubation of the viral vector particle is carried out after the spinoculation. In some embodiments, the at least a portion of the incubation of the viral vector particle that is carried out after the spinoculation is carried out for no more than or no more than about 2 hours, 4 hours, 12 hours, 18 hours, 24 hours, 30 hours, 36 hours, 48 hours, 60 hours, or 72 hours. In some embodiments, the at least a portion of the incubation of the viral vector particle that is carried out after the spinoculation is carried out for or for about 24 hours.

In some embodiments, the total duration of the incubation of the viral vector particle is for no more than 12 hours, 24 hours, 36 hours, 48 hours, or 72 hours.

In some embodiments, the incubation of the cells with the viral vector particles results in or produces an output composition comprising cells transduced with the viral vector particles, which is also referred to herein as a population of transduced cells. Accordingly, in some embodiments, the population of transduced cells comprises T cells transduced with the heterologous polynucleotide. In some embodiments, at least 20%, at least 25%, at least 30%, at least 35%, at least 40%, at least 45%, at least 50%, at least 55%, at least 60%, at least 65%, at least 70%, at least 75%, at least 80%, or at least 85% of the T cells in the population of transduced cells are transduced with the heterologous polynucleotide. In some embodiments, at least 55%, at least 60%, at least 65%, at least 70%, at least 75%, at least 80%, or at least 85% of the T cells in the population of transduced cells are transduced with the heterologous polynucleotide. In some embodiments, at least 95%, at least 96%, at least 97%, at least 98%, or at least 99% of the T cells transduced with the heterologous polynucleotide are CCR7+.

In some embodiments, the population of transduced cells comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% cells that express the recombinant protein. In some embodiments, the population of transduced cells comprises at least 55%, at least 60%, at least 65%, at least 70%, at least 75%, at least 80%, at least 85%, at least 90%, or at least 95% cells that express the recombinant protein.

The percentage of cells in the population of transduced cells produced by a method as described here that includes selecting for CCR7+ T cells, or otherwise transducing a population of T cells enriched in CCR7+ T cells, e.g., as described in Section I-A, can be compared to the percentage of cells in a population of transduced cells produced by the same method except for lacking the step of enriching for CCR7+ T cells. In some embodiments, the percentage of cells in the population of transduced cells is at least 0.5-fold, at least 1-fold, at least 1.5-fold, or at least 2-fold greater as compared to a cell composition that was not enriched for CCR7+ primary T cells through a selection step. In some embodiments, the percentage of cells in the population of transduced cells is at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% greater as compared to a cell composition that was not enriched for CCR7+ primary T cells through a selection step. For instance, if the percentage of cells in the population of transduced cells is 80% and the percentage of cells in a cell population that was not enriched for CCR7+ primary T cells is 40%, then the percentage of cells in the population of transduced cells is 100% greater as compared to the cell population that was not enriched for CCR7+ primary T cells. In some embodiments, the percentage of cells in the population of transduced cells expressing the recombinant protein is at least 0.5-fold, at least 1-fold, at least 1.5-fold, or at least 2-fold greater as compared to a cell composition that was not enriched for CCR7+ primary T cells through a selection step. In some embodiments, the percentage of cells in the population of transduced cells expressing the recombinant protein is at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% greater as compared to a cell composition that was not enriched for CCR7+ primary T cells through a selection step.

In some embodiments, the method further comprises one or more additional steps. In some embodiments, the method further comprises recovering or isolating from the population of transduced cells the transduced cells produced by the method. In some embodiments, the recovering or isolating comprises selecting for expression of the recombinant protein (e.g., the CAR or TCR).

The percentage of T cells in the population of transduced cells that are transduced with the heterologous polynucleotide can be compared to the percentage of transduced T cells in other populations of transduced cells, e.g., the percentage of T cells in a plurality of populations of transduced cells that are transduced with a heterologous polynucleotide can be compared. In some embodiments, the maximum variability among the percentage of transduced T cells in the plurality of populations is less than 50%, less than 40%, less than 30%, less than 25%, less than 20%, less than 15%, less than 10%, or less than 5%, from the average percentage of transduction among the plurality. For example, a plurality of populations of transduced cells that include transduction percentages of 70%, 80%, and 90%, has a maximum variability of 12.5%. In some embodiments, the plurality of populations of transduced cells includes at least 5, at least 10, at least 20, at least 30, at least 40, at least 50, at least 60, at least 70, at least 80, at least 90, or at least 100 populations of transduced cells.

E. Further Cultivation and Expansion

In some embodiments, the provided methods include one or more steps for cultivating engineered cells, e.g., cultivating cells under conditions that promote proliferation and/or expansion. In certain embodiments, the provided methods do not include steps for cultivating engineered cells. In certain embodiments, there is a greater number of engineered cells following the completion of the process as compared to the initial source cells from which the cells were generated. In various embodiments, there is a smaller number of engineered cells following the completion of the process as compared to the initial source cells from which the cells were generated. In some embodiments, engineered cells are cultivated under conditions that promote proliferation and/or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a polynucleotide, e.g., a heterologous polynucleotide encoding a recombinant protein. In some embodiments, the cells that are cultivated are cells of the population of transduced cells, e.g., as described in Section I-D. In some embodiments, the cells that are cultivated are cells of a population of transduced cells produced by any of the methods provided herein. In some embodiments, the cultivation produces an output composition containing a composition of enriched T cells that express the recombinant receptor (e.g., CAR).

In some embodiments, the engineered cells are cultured in a container that can be filled, e.g., via the feed port, with cell media and/or cells for culturing of the added cells. The cells can be from any cell source for which culture of the cells is desired, for example, for expansion and/or proliferation of the cells.

In some aspects, the culture media is an adapted culture medium that supports that growth, cultivation, expansion or proliferation of the cells, such as T cells. In some aspects, the medium can be a liquid containing a mixture of salts, amino acids, vitamins, sugars or any combination thereof. In some embodiments, the culture media further contains one or more stimulating conditions or agents, such as to stimulate the cultivation, expansion or proliferation of cells during the incubation. In some embodiments, the stimulating condition is or includes one or more cytokine selected from IL-2, IL-7, or IL-15. In some embodiments, the cytokine is a recombinant cytokine. In some embodiments, the concentration of the one or more cytokine in the culture media during the culturing or incubation, independently, is from or from about 1 IU/mL to 1500 IU/mL, such as from or from about 1 IU/mL to 100 IU/mL, 2 IU/mL to 50 IU/mL, 5 IU/mL to 10 IU/mL, 10 IU/mL to 500 IU/mL, 50 IU/mL to 250 IU/mL or 100 IU/mL to 200 IU/mL, 50 IU/mL to 1500 IU/mL, 100 IU/mL to 1000 IU/mL, or 200 IU/mL to 600 IU/mL. In some embodiments, the concentration of the one or more cytokine, independently, is at least or at least about 1 IU/mL, 5 IU/mL, 10 IU/mL, 50 IU/mL, 100 IU/mL, 200 IU/mL, 500 IU/mL, 1000 IU/mL, or 1500 IU/mL.

In some aspects, the cells are incubated for at least a portion of time after transfer of the engineered cells and culture media. In some embodiments, the stimulating conditions generally include a temperature suitable for the growth of primary immune cells, such as human T lymphocytes, for example, at least about 25 degrees Celsius, generally at least about 30 degrees, and generally at or about 37 degrees Celsius. In some embodiments, the cells are incubated at a temperature of 25 to 38 degrees Celsius, such as 30 to 37 degrees Celsius, for example at or about 37 degrees Celsius±2 degrees Celsius. In some embodiments, the incubation is carried out for a time period until the culture, e.g., cultivation or expansion, results in a desired or threshold density, number or dose of cells. In some embodiments, the incubation is greater than or greater than about or is for about or 24 hours, 48 hours, 72 hours, 96 hours, 5 days, 6 days, 7 days, 8 days, or 9 days or more.

In some embodiments, the cells are incubated under conditions to maintain a target amount of carbon dioxide in the cell culture. In some aspects, this ensures optimal cultivation, expansion and proliferation of the cells during the growth. In some aspects, the amount of carbon dioxide (CO₂) is between 10% and 0% (v/v) of said gas, such as between 8% and 2% (v/v) of said gas, for example an amount of or about 5% (v/v) C02.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g., for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In some embodiments, cells are incubated using containers, e.g., bags, which are used in connection with a bioreactor. In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. In some embodiments, at least a portion of the incubation is carried out with rocking. The rocking speed and rocking angle may be adjusted to achieve a desired agitation. In some embodiments the rock angle is or is about 20°, 19°, 18°, 17°, 16°, 15°, 14°, 13°, 12°, 110, 10°, 9°, 8°, 7° 6°, 5° 4°, 3°, 2°, or 1°. In certain embodiments, the rock angle is between 6-16°. In other embodiments, the rock angle is between 7-16°. In other embodiments, the rock angle is between 8-12°. In some embodiments, the rock rate is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 rpm. In some embodiments, the rock rate is between 4 and 12 rpm, such as between 4 and 6 rpm, inclusive. At least a portion of the cell culture expansion is performed with a rocking motion, such as at an angle of between 5° and 10°, such as 6°, at a constant rocking speed, such as a speed of between 5 and 15 RPM, such as 6 RMP or 10 RPM. The CD4+ and CD8+ cells are each separately expanded until they each reach a threshold amount or cell density.

In some embodiments, at least a portion of the incubation is carried out under static conditions. In some embodiments, at least a portion of the incubation is carried out with perfusion, such as to perfuse out spent media and perfuse in fresh media during the culture. In some embodiments, the method includes a step of perfusing fresh culture medium into the cell culture, such as through a feed port. In some embodiments, the culture media added during perfusion contains the one or more stimulating agents, e.g., one or more recombinant cytokine, such as IL-2, IL-7, and/or IL-15. In some embodiments, the culture media added during perfusion is the same culture media used during a static incubation.

In some embodiments, the cells are expanding or cultivated in the presence of one or more anti-idiotype antibodies, such as an anti-idiotypic antibody that binds to or recognizes the recombinant receptor that is expressed by the engineered cells.

In some embodiments, subsequent to the incubation, a container, e.g., bag, is re-connected to a system for carrying out the one or more other processing steps of for manufacturing, generating or producing the cell therapy, such as is re-connected to the system containing the centrifugal chamber. In some aspects, cultured cells are transferred from the bag to the internal cavity of the chamber for formulation of the cultured cells.

### II. Compositions

Also provided herein is a composition comprising a population of transduced cells produced by any of the methods provided herein.

In some embodiments, provided herein are therapeutic compositions (e.g., therapeutic T cell compositions) generated a method including the transduction method disclosed herein, e.g., an output composition, such as those disclosed in Section I-D or Section I-E. In some embodiments, the therapeutic composition includes a population of transduced cells, as described in, e.g., Section I-D. In some embodiments, provided herein are therapeutic compositions (e.g., therapeutic T cell compositions) having any one or more of the features disclosed herein. In some embodiments, the dose of cells comprising cells engineered with a recombinant antigen receptor, e.g. CAR or TCR, is provided as a composition or formulation, such as a pharmaceutical composition or formulation. Such compositions can be used in accord with the provided methods, and/or with the provided compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cells are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution. In some embodiments, the cells are frozen, e.g., cryoprotected or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryoprotected or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the composition of enriched T cells, e.g., T cells that have been selected, stimulated, engineered, and/or cultivated, are formulated, cryoprotected, and then stored for an amount of time. In certain embodiments, the formulated, cryoprotected cells are stored until the cells are released for infusion. In particular embodiments, the formulated cryoprotected cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryoprotected and stored for, for about, or for less than 1 days, 2 days, 3 days, 4 days, 5 days, 6 days, or 7 days. In certain embodiments, the cells are thawed and administered to a subject after the storage. In certain embodiments, the cells are stored for or for about 5 days. In some embodiments, the formulated cells are not cryopreserved.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

Also provided are articles of manufacture or kits, including (i) any composition described herein; and (ii) instructions for administering the composition to a subject.

In some embodiments, the articles of manufacture or kits include one or more containers, typically a plurality of containers, packaging material, and a label or package insert on or associated with the container or containers and/or packaging, generally including instructions for use. In some embodiments, the articles of manufacture and kits contain engineered cells expressing a recombinant receptor or compositions thereof, such as those generated using the methods provided herein, and optionally instructions for use, for example, instructions for administering. In some embodiments, the instructions provide directions or specify methods for assessing if a subject, prior to receiving a cell therapy, is likely or suspected of being likely to respond and/or the degree or level of response following administration of engineered cells expressing a recombinant receptor for treating a disease or disorder. In some aspects, the articles of manufacture can contain a dose or a composition of engineered cells.

The articles of manufacture provided herein contain packaging materials. Packaging materials for use in packaging the provided materials are well known to those of skill in the art. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the reagents and/or cell compositions are packaged separately. In some embodiments, each container can have a single compartment. In some embodiments, other components of the articles of manufacture or kits are packaged separately, or together in a single compartment.

### III. Methods of Treatment and Uses

Provided herein are methods of treatment, e.g., including administering any of the engineered cells or compositions containing engineered cells described herein. In some aspects, also provided are methods of administering any of the engineered cells or compositions containing engineered cells described herein to a subject, such as a subject that has a disease or disorder. In some aspects, also provided are uses of any of the engineered cells or compositions containing engineered cells described herein for treatment of a disease or disorder. In some aspects, also provided are uses of any of the engineered cells or compositions containing engineered cells described herein for the manufacture of a medicament for the treatment of a disease or disorder. In some aspects, also provided are any of the engineered cells or compositions containing engineered cells described herein, for use in treatment of a disease or disorder, or for administration to a subject having a disease or disorder.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described above. In particular embodiments, the chimeric antigen receptor or transgenic TCR specifically binds to an antigen associated with the disease or condition.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen_{1B} (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Ra), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT) vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is or includes a pathogen-specific or pathogen-expressed antigen, such as a viral antigen (e.g., a viral antigen from HIV, HCV, HBV), bacterial antigens, and/or parasitic antigens.

In some embodiments, the antibody or an antigen-binding fragment of the CAR (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, 10 million cells, about 15 million cells, about 20 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, the dose of cells is a flat dose of cells or fixed dose of cells such that the dose of cells is not tied to or based on the body surface area or weight of a subject.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸ total such cells, or the range between any two of the foregoing values.

In some embodiments, the dose of genetically engineered cells comprises from or from about 1×10⁵ to 5×10⁸ total CAR-expressing T cells, 1×10⁵ to 2.5×10⁸ total CAR-expressing T cells, 1×10⁵ to 1×10⁸ total CAR-expressing T cells, 1×10⁵ to 5×10⁷ total CAR-expressing T cells, 1×10⁵ to 2.5×10⁷ total CAR-expressing T cells, 1×10⁵ to 1×10⁷ total CAR-expressing T cells, 1×10⁵ to 5×10⁶ total CAR-expressing T cells, 1×10⁵ to 2.5×10⁶ total CAR-expressing T cells, 1×10⁵ to 1×10⁶ total CAR-expressing T cells, 1×10⁶ to 5×10⁸ total CAR-expressing T cells, 1×10⁶ to 2.5×10⁸ total CAR-expressing T cells, 1×10⁶ to 1×10⁸ total CAR-expressing T cells, 1×10⁶ to 5×10⁷ total CAR-expressing T cells, 1×10⁶ to 2.5×10⁷ total CAR-expressing T cells, 1×10⁶ to 1×10⁷ total CAR-expressing T cells, 1×10⁶ to 5×10⁶ total CAR-expressing T cells, 1×10⁶ to 2.5×10⁶ total CAR-expressing T cells, 2.5×10⁶ to 5×10⁸ total CAR-expressing T cells, 2.5×10⁶ to 2.5×10⁸ total CAR-expressing T cells, 2.5×10⁶ to 1×10⁸ total CAR-expressing T cells, 2.5×10⁶ to 5×10⁷ total CAR-expressing T cells, 2.5×10⁶ to 2.5×10⁷ total CAR-expressing T cells, 2.5×10⁶ to 1×10⁷ total CAR-expressing T cells, 2.5×10⁶ to 5×10⁶ total CAR-expressing T cells, 5×10⁶ to 5×10⁸ total CAR-expressing T cells, 5×10⁶ to 2.5×10⁸ total CAR-expressing T cells, 5×10⁶ to 1×10⁸ total CAR-expressing T cells, 5×10⁶ to 5×10⁷ total CAR-expressing T cells, 5×10⁶ to 2.5×10⁷ total CAR-expressing T cells, 5×10⁶ to 1×10⁷ total CAR-expressing T cells, 1×107 to 5×10⁸ total CAR-expressing T cells, 1×10⁷ to 2.5×10⁸ total CAR-expressing T cells, 1×10⁷ to 1×10⁸ total CAR-expressing T cells, 1×10⁷ to 5×10⁷ total CAR-expressing T cells, 1×10⁷ to 2.5×10⁷ total CAR-expressing T cells, 2.5×10⁷ to 5×10⁸ total CAR-expressing T cells, 2.5×10⁷ to 2.5×10⁸ total CAR-expressing T cells, 2.5×10⁷ to 1×10⁸ total CAR-expressing T cells, 2.5×10⁷ to 5×10⁷ total CAR-expressing T cells, 5×10⁷ to 5×10⁸ total CAR-expressing T cells, 5×10⁷ to 2.5×10⁸ total CAR-expressing T cells, 5×10⁷ to 1×10⁸ total CAR-expressing T cells, 1×10⁸ to 5×10⁸ total CAR-expressing T cells, 1×10⁸ to 2.5×10⁸ total CAR-expressing T cells, or 2.5×10⁸ to 5×10⁸ total CAR-expressing T cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8+, in some cases also recombinant receptor-expressing (e.g. CAR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/CAR+ or CD8+/CAR+ cells, from or from about 5×10⁵ to 1×10⁷ total CD3+/CAR+ or CD8+/CAR+ cells, or from or from about 1×10⁶ to 1×10⁷ total CD3+/CAR+ or CD8+/CAR+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷, 1×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8+ and CD4+ T cells, respectively, and/or CD8+− and CD4+− enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

In some embodiments, the methods comprise administration of a chemotherapeutic agent, e.g., a conditioning chemotherapeutic agent, for example, to reduce tumor burden prior to the administration.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the first or subsequent dose.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known. See, for instance, Wadwa et al., J. Drug Targeting 3: 1 1 1 (1995), and U.S. Pat. No. 5,087,616.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agent includes a cytokine, such as IL-2, for example, to enhance persistence.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se.

As used herein, a subject includes any living organism, such as humans and other mammals. Mammals include, but are not limited to, humans, and non-human animals, including farm animals, sport animals, rodents, and pets. In particular embodiments, a subject is a human subject.

As used herein, “depleting” when referring to one or more particular cell type or cell population, refers to decreasing the number or percentage of the cell type or population, e.g., compared to the total number of cells in or volume of the composition, or relative to other cell types, such as by negative selection based on markers expressed by the population or cell, or by positive selection based on a marker not present on the cell population or cell to be depleted. The term does not require complete removal of the cell, cell type, or population from the composition.

As used herein, “enriching” when referring to one or more particular cell type or cell population, refers to increasing the number or percentage of the cell type or population (e.g., CCR7+ cells), e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” or “+” for a particular marker refers to the detectable presence on or in the cell of a particular marker, typically a surface marker. When referring to a surface marker, the term refers to the presence of surface expression as detected, in some embodiments, by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected, in some embodiments, by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acids generally can be grouped according to the following common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative amino acid substitutions will involve exchanging a member of one of these classes for another class.

As used herein, “at a position corresponding to” or recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.,: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Vectors include viral vectors, such as retroviral vectors, for example lentiviral or gammaretroviral vectors, having a genome carrying another nucleic acid and capable of inserting into a host genome for propagation thereof.

As used herein, a composition refers to any mixture of two or more products, substances, or compounds, including cells. It may be a solution, a suspension, liquid, powder, a paste, aqueous, non-aqueous or any combination thereof.

As used herein, the terms “treatment,” “treat,” and “treating,” refer to complete or partial amelioration or reduction of a disease or condition or disorder, or a symptom, adverse effect or outcome, or phenotype associated therewith. In certain embodiments, the effect is therapeutic, such that it partially or completely cures a disease or condition or adverse symptom attributable thereto.

As used herein, a “therapeutically effective amount” of a compound or composition or combination refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered.

### V. Exemplary Embodiments

Among the provided embodiments are:

1. A method for increasing transduction frequency of primary T cells, the method comprising: (a) selecting primary T cells that are positive for surface expression of CCR7 from a biological sample comprising a population of primary T cells, thereby generating an input population enriched in CCR7+ primary T cells; (b) incubating the input population under stimulatory conditions, thereby generating a stimulated composition, wherein said stimulating conditions comprise the presence of a stimulatory reagent capable of activating one or more intracellular signaling domains of one or more components of a TCR complex and/or one or more intracellular signaling domains of one or more costimulatory molecules; and (c) incubating a viral vector particle comprising a heterologous polynucleotide encoding a recombinant protein with T cells of the stimulated composition, thereby generating a population of transduced cells.

2. A method for increasing transduction frequency of primary T cells, the method comprising: (a) selecting primary T cells that are positive for surface expression of CCR7 from a biological sample comprising a population of primary T cells, thereby generating an input population enriched in CCR7+ primary T cells; and (b) incubating a viral vector particle comprising a heterologous polynucleotide encoding a recombinant protein with T cells of the input population of cells, thereby generating a population of transduced cells.

3. A method for increasing transduction frequency of primary T cells, the method comprising: (a) selecting primary T cells that are positive for surface expression of CCR7 from a biological sample comprising a population of primary T cells, thereby generating an input population enriched in CCR7+ primary T cells; (b) optionally, incubating the input population under stimulatory conditions, thereby generating a stimulated composition, wherein said stimulating conditions comprise the presence of a stimulatory reagent capable of activating one or more intracellular signaling domains of one or more components of a TCR complex and/or one or more intracellular signaling domains of one or more costimulatory molecules; and (c) a viral vector particle comprising a heterologous polynucleotide encoding a recombinant protein with T cells of the input population of cells, or optionally of the stimulated composition, thereby generating a population of transduced cells.

4. A method for increasing transduction frequency of primary T cells, the method comprising: (a) incubating an input population of primary T cells enriched in CCR7+ T cells under stimulatory conditions, thereby generating a stimulated composition, wherein said stimulating conditions comprise the presence of a stimulatory reagent capable of activating one or more intracellular signaling domains of one or more components of a TCR complex and/or one or more intracellular signaling domains of one or more costimulatory molecules; and (b) incubating a viral vector particle comprising a heterologous polynucleotide encoding a recombinant protein with T cells of the stimulated composition, thereby generating a population of transduced cells.

5. A method for increasing transduction frequency of primary T cells, the method comprising incubating a viral vector particle comprising a heterologous polynucleotide encoding a recombinant protein with T cells of an input population of primary T cells enriched in CCR7+ T cells, thereby generating a population of transduced cells.

6. The method of any one of embodiments 1-5, wherein at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the input population are CCR7+ primary T cells.

7. The method of any one of embodiments 1-6, wherein at least 90%, at least 91%, at least 92%, at least 93%, at least 94%, at least 95%, at least 96%, at least 97%, at least 98%, at least 99%, or 100% of the input population are CCR7+ primary T cells.

8. The method of any one of embodiments 1-7, wherein at least 95%, at least 96%, at least 97%, at least 98%, at least 99%, or 100% of the input population are CCR7+ primary T cells.

9. The method of any one of embodiments 1-3 and 6, wherein the selecting does not comprise selecting for cells that are (a) CCR7+ and CD45RO+; or (b) CCR7+ and CD27+; or (c) CCR7+ and CD45RA−; or (d) CCR7+ and CD62L+; or (e) CCR7+ and CD45RA+; or (f) CCR7+ and CD62L−.

10. The method of any one of embodiments 1-9, wherein the input population is not enriched in T cells that are (a) CCR7+ and CD45RO+; or (b) CCR7+ and CD27+; or (c) CCR7+ and CD45RA−; or (d) CCR7+ and CD62L+; or (e) CCR7+ and CD45RA+; or (f) CCR7+ and CD62L−.

11. The method of any one of embodiments 1-10, wherein the input population is not enriched in CCR7+ and CD45RO+ T cells, optionally wherein less than 85% of the total cells of the input population are CCR7+ and CD45RO+ T cells.

12. The method of any one of embodiments 1-11, wherein the input population is not enriched in CCR7+ and CD27+ T cells, optionally wherein less than 85% of the total cells of the input population are CCR7+ and CD27+ T cells.

13. The method of any one of embodiments 1-12, wherein the input population is not enriched in CCR7+ and CD45RA− T cells, optionally wherein less than 85% of the total cells of the input population are CCR7+ and CD45RA− T cells.

14. The method of any one of embodiments 1-13, wherein the input population is not enriched in CCR7+ and CD62L+ T cells, optionally wherein less than 85% of the total cells of the input population are CCR7+ and CD62L+ T cells.

15. The method of any one of embodiments 1-14, wherein the input population is not enriched in CCR7+ and CD45RA+ T cells, optionally wherein less than 85% of the total cells of the input population are CCR7+ and CD45RA+ T cells.

16. The method of any one of embodiments 1-15, wherein the input population is not enriched in CCR7+ and CD62L− T cells, optionally wherein less than 85% of the total cells of the input population are CCR7+ and CD62L− T cells.

17. The method of any one of embodiments 1-3 and 6-16, wherein the biological sample is a blood sample.

18. The method of any one of embodiments 1-3 and 6-16, wherein the biological sample is a leukapheresis sample.

19. The method of any one of embodiments 1-18, wherein the T cells are unfractionated T cells, are enriched or isolated CD3+ T cells, are enriched or isolated CD4+ T cells, or are enriched or isolated CD8+ T cells.

20. The method of any one of embodiments 1-19, wherein the input population comprises at least 80%, at least 85%, at least 90%, or at least 95% cells that are CD4+ T cells or CD8+ T cells.

21. The method of any one of embodiments 1-20, wherein the input population comprises at least 80%, at least 85%, at least 90%, or at least 95% cells that are CD4+ T cells.

22. The method of any one of embodiments 1-20, wherein the input population comprises at least 80%, at least 85%, at least 90%, or at least 95% cells that are CD8+ T cells.

23. The method of any one of embodiments 1-20, wherein the input population comprises at least 80%, at least 85%, at least 90%, or at least 95% cells that are CD4+ T cells and CD8+ T cells.

24. The method of embodiment 23, wherein the ratio of the CD4+ T cells to the CD8+ T cells is or is about 1:1, 1:2, 2:1, 1:3, or 3:1.

25. The method of any one of embodiments 1-19, wherein the input population comprises at least 80%, at least 85%, at least 90%, or at least 95% cells that are CD3+ T cells.

26. The method of any one of embodiments 1-25, wherein the input population comprises between 100×10⁶ and 500×10⁶ total T cells.

27. The method of any one of embodiments 1-26, wherein the input population comprises between 200×10⁶ and 400×10⁶ total T cells, optionally at or about 300×10⁶ total T cells.

28. The method of embodiment 26 or embodiment 27, wherein the total T cells are viable T cells.

29. The method of any one of embodiments 1-28 wherein at least 10%, at least 20%, at least 30%, at least 40%, at least 50%, or at least 60% of the cells of the stimulated composition: (i) express a surface marker selected from the group consisting of HLA-DR, CD25, CD69, CD71, CD40L, and 4-1BB; (ii) comprise intracellular expression of a cytokine selected from the group consisting of IL-2, IFN-gamma, and TNF-alpha; (iii) are in the G1 or later phase of the cell cycle; and/or (iv) are capable of proliferating.

30. The method of any one of embodiments 1-29 wherein the stimulatory reagent comprises a primary agent that specifically binds to a member of a TCR complex, optionally that specifically binds to CD3.

31. The method of embodiment 30, wherein the stimulatory reagent further comprises a secondary agent that specifically binds to a T cell costimulatory molecule, optionally wherein the costimulatory molecule is selected from CD28, CD137 (4-1-BB), OX40, or ICOS.

32. The method of embodiment 30 or embodiment 31, wherein the primary and/or secondary agents comprise an antibody, optionally wherein the stimulatory reagent comprises incubation with an anti-CD3 antibody and an anti-CD28 antibody, or an antigen-binding fragment thereof.

33. The method of any one of embodiments 30-32, wherein the primary agent and/or secondary agent are present on the surface of a solid support.

34. The method of embodiment 33, wherein the solid support is or comprises a bead.

35. The method of any of embodiments 30-34, wherein the primary agent and secondary agent are reversibly bound on the surface of an oligomeric particle reagent comprising a plurality of streptavidin or streptavidin mutein molecules.

36. The method of embodiment 35, wherein each of the plurality of the streptavidin or streptavidin mutein molecules comprise the amino acid sequence of Val44-Thr⁴⁵-Ala⁴⁶-Arg⁴⁷ or Ile⁴⁴-Gly⁴⁵-Ala⁴⁶-Arg⁴⁷ at sequence positions corresponding to positions 44 to 47 with reference to positions in streptavidin in the sequence of amino acids set forth in SEQ ID NO: 34.

37. The method of embodiment 35, wherein each of the plurality of the streptavidin or streptavidin mutein molecules are streptavidin mutein molecules, and wherein each of the plurality of the streptavidin mutein molecules is or comprises: a) the sequence of amino acids set forth in any of SEQ ID NOS: 36, 41, 48-50, or 53-55; b) a sequence of amino acids that exhibit at least 85%, 86%, 87%, 88%, 89%, 89%, 90%, 91%, 92%, 93%, 94%, 95%, 96%, 97%, 98%, or 99% or more sequence identity to any of SEQ ID NOS: 36, 41, 48-50, or 53-55 and contain the amino acid sequence corresponding to Val44-Thr45-Ala46-Arg47 or Ile44-Gly45-Ala46-Arg47 and/or reversibly bind to biotin, a biotin analog or a streptavidin-binding peptide; or c) a functional fragment of a) or b) that reversibly binds to biotin, a biotin analog, or a streptavidin-binding peptide, optionally wherein each of the plurality of the streptavidin mutein molecules is or comprises the amino acid sequence set forth in SEQ ID NO:36 or SEQ ID NO:41.

38. The method of any one of embodiments 1-37, wherein the population of transduced cells comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% cells that express the recombinant protein.

39. The method of any one of embodiments 1-38, wherein the population of transduced cells comprises at least 55%, at least 60%, at least 65%, at least 70%, at least 75%, at least 80%, at least 85%, at least 90%, or at least 95% cells that express the recombinant protein.

40. The method of any one of embodiments 1-39, wherein the percentage of cells in the population of transduced cells expressing the recombinant protein is at least 0.5-fold, at least 1-fold, at least 1.5-fold, or at least 2-fold greater as compared to a cell composition that was not enriched for CCR7+ primary T cells through a selection step.

41. The method of any of embodiments 1-40, wherein the incubating the viral vector particle comprises a step of spinoculating the viral vector particles with the input population or the stimulated composition.

42. The method of embodiment 41, wherein spinoculating comprises rotating, in an internal cavity of a centrifugal chamber, the viral vector particles and the input population or stimulated composition, wherein the rotation is at a relative centrifugal force at an internal surface of the side wall of the cavity that is: between or between about 500 g and 2500 g, 500 g and 2000 g, 500 g and 1600 g, 500 g an 1000 g, 600 g and 1600 g, 600 g and 1000 g, 1000 g and 2000 g, or 1000 g and 1600 g, each inclusive; or at least or at least about 600 g, 800 g, 1000 g, 1200 g, 1600 g, or 2000 g.

43. The method of embodiment 41 or embodiment 42, wherein spinoculating is for a time that is: greater than or about 5 minutes, greater than or about 10 minutes, greater than or about 15 minutes, greater than or about 20 minutes, greater than or about 30 minutes, greater than or about 45 minutes, greater than or about 60 minutes, greater than or about 90 minutes, or greater than or about 120 minutes; or between or between about 5 minutes and 60 minutes, 10 minutes and 60 minutes, 15 minutes and 60 minutes, 15 minutes and 45 minutes, 30 minutes and 60 minutes, or 45 minutes and 60 minutes, each inclusive.

44. The method of any of embodiments 1-43, further comprising during at least a portion of the incubating contacting the stimulated composition and/or viral vector particles with a transduction adjuvant.

45. The method of any of embodiments 2, 3, and 5-44, further comprising, during at least a portion of the incubating, contacting the input population and/or viral vector particles with a transduction adjuvant.

46. The method of embodiment 44, wherein the contacting is carried out prior to, concomitant with, or after the spinoculating the viral vector particles with the input population or the stimulated composition.

47. The method of any of embodiments 1-46, wherein at least a portion of the incubation of the viral vector particle is carried out at or about 37° C.±2° C.

48. The method of any of embodiments 1-47, wherein at least a portion of the incubation of the viral vector particle is carried out after the spinoculation.

49. The method of embodiment 47 or embodiment 48, wherein the at least a portion of the incubation of the viral vector particle is carried out for no more than or no more than about 2 hours, 4 hours, 12 hours, 18 hours, 24 hours, 30 hours, 36 hours, 48 hours, 60 hours, or 72 hours.

50. The method of any of embodiments 47-49, wherein the at least a portion of the incubation of the viral vector particle is carried out for or for about 24 hours.

51. The method of any of embodiments 1-50, wherein the total duration of the incubation of the viral vector particle is for no more than 12 hours, 24 hours, 36 hours, 48 hours, or 72 hours.

52. The method of any of embodiments 1-51, wherein the viral vector particle is a lentiviral vector particle.

53. The method of embodiment 52, wherein the lentiviral vector particle is replication defective.

54. The method of any of embodiments 1-53, wherein the viral vector particle is pseudotyped with a viral envelope glycoprotein.

55. The method of embodiment 54, wherein the viral envelope glycoprotein is VSV-G.

56. The method of any of embodiments 1-55, wherein the viral vector particle is incubated at a multiplicity of infection of less than or less than about 20.0 or less than or less than about 10.0.

57. The method of any of embodiments 1-56, wherein: the viral vector particle is incubated at a multiplicity of infection from or from about 1.0 IU/cell to 10 IU/cell, or 2.0 U/cell to 5.0 IU/cell; or the viral vector particle is incubated at a multiplicity of infection of at least or at least about 1.6 IU/cell, 1.8 IU/cell, 2.0 IU/cell, 2.4 IU/cell, 2.8 IU/cell, 3.2 IU/cell, 3.6 IU/cell, 4.0 IU/cell, 5.0 IU/cell, 6.0 IU/cell, 7.0 IU/cell, 8.0 IU/cell, 9.0 IU/cell, or 10.0 IU/cell.

58. The method of any of embodiments 1-57 wherein the stimulated composition comprises at least at or about at least or about 50×10⁶ cells, 100×10⁶ cells, or 200×10⁶ cells.

59. The method of any of embodiments 1, 3, 4, and 6-58, wherein the stimulated composition comprises between at or about 50×10⁶ cells and at or about 300×10⁶ cells, inclusive, optionally between at or about 100×10⁶ cells and at or about 200×10⁶ cells, inclusive.

60. The method of any of embodiments 2 and 6-59, wherein the input population comprises at least at or about at least or about 50×10⁶ cells, 100×10⁶ cells, or 200×10⁶ cells.

61. The method of any of embodiments 2 and 6-59, wherein the input population comprises between at or about 50×10⁶ cells and at or about 300×10⁶ cells, inclusive, optionally between at or about 100×10⁶ cells and at or about 200×10⁶ cells, inclusive.

62. The method of any of embodiments 1-61, wherein the T cells incubated with the viral particle comprises at least at or about at least or about 50×10⁶ cells, 100×10⁶ cells, or 200×10⁶ cells.

63. The method of any of embodiments 1-61, wherein the T cells incubated with the viral particle comprises between at or about 50×10⁶ cells and at or about 300×10⁶ cells, inclusive, optionally between at or about 100×10⁶ cells and at or about 200×10⁶ cells, inclusive

64. The method of any of embodiments 1-63, wherein the recombinant protein is an antigen receptor.

65. The method of embodiment 64, wherein the antigen receptor is a transgenic T cell receptor (TCR).

66. The method of embodiment 64, wherein the antigen receptor is a chimeric antigen receptor (CAR).

67. The method of embodiment 66, wherein the CAR comprises an extracellular antigen-recognition domain that specifically binds to a target antigen, an intracellular signaling domain comprising an ITAM, and a transmembrane domain linking the extracellular domain and the intracellular signaling domain.

68. The method of embodiment 67, wherein the intracellular signaling domain comprises an intracellular domain of a CD3-zeta (CD3ζ) chain.

69. The method of embodiment 67 or embodiment 68, further comprising a transmembrane domain linking the extracellular domain and the intracellular signaling domain.

70. The method of embodiment 69, wherein the transmembrane domain comprises a transmembrane portion of CD28.

71. The method of any of embodiments 67-70, wherein the intracellular signaling domain further comprises an intracellular signaling domain of a T cell costimulatory molecule.

72. The method of embodiment 71, wherein the T cell costimulatory molecule is selected from the group consisting of CD28 and 41BB.

73. The method of any of embodiments 64-72, wherein the antigen receptor specifically binds to an antigen associated with a disease or condition or specifically binds to a universal tag.

74. The method of embodiment 73, wherein the disease or condition is a cancer, an autoimmune disease or disorder, or an infectious disease.

75. The method of any of embodiments 1-74, wherein the population of transduced cells comprises T cells transduced with the heterologous polynucleotide.

76. The method of embodiment 75, wherein at least 20%, at least 25%, at least 30%, at least 35%, at least 40%, at least 45%, at least 50%, at least 55%, at least 60%, at least 65%, at least 70%, at least 75%, at least 80%, or at least 85% of the T cells in the population of transduced cells are transduced with the heterologous polynucleotide.

77. The method of embodiment 75 or embodiment 76, wherein at least 55%, at least 60%, at least 65%, at least 70%, at least 75%, at least 80%, or at least 85% of the T cells in the population of transduced cells are transduced with the heterologous polynucleotide.

78. The method of any one of embodiments 75-77, wherein at least 95%, at least 96%, at least 97%, at least 98%, or at least 99% of the T cells transduced with the heterologous polynucleotide are CCR7+.

79. The method of embodiment 75 or embodiment 76, further comprising recovering or isolating from the population of transduced cells the transduced T cells produced by the method.

80. The method of any one of embodiments 1-79, wherein, among a plurality of populations of transduced cells, the percentage of T cells in the population of transduced cells that are transduced with the heterologous polynucleotide varies by 30% or less, 25% or less, 20% or less, 15% or less, or 10% or less.

81. The method of any of embodiments 1-80, that is carried out in vitro or ex vivo.

82. A composition comprising a population of transduced cells produced by the method of any one of embodiments 1-81.

83. The composition of embodiment 82, further comprising a cyropreservant.

### VI. Examples

The following examples are included for illustrative purposes only and are not intended to limit the scope of the invention.

### Example 1: Assessment of Transduction Frequency Among Donor T Cells

Primary CD4+ and CD8+ T cells were selected from isolated PBMCs from leukapheresis samples from plurality of human subjects with relapsed/refractory large B-cell lymphoma. The CD4+ and CD8+ T cells (about 300×10⁶ cells each) were separately stimulated in the presence of paramagnetic beads conjugated with anti-CD3 and anti-CD28 antibodies at about a 1:1 ratio of beads to cells, in the presence of recombinant IL-2, IL-7, and IL-15. The stimulation was carried out by incubation for between 18 to 30 hours. The stimulated cells were then incubated with a target volume of lentiviral vector encoding a heterologous nucleic acid (in this example encoding a chimeric antigen receptor (CAR) directed against a specific antigen (e.g., CD19)) and transduced via spinoculation. The cells were transduced in the presence of 10 μg/ml protamine sulfate. After spinoculation, the cells were incubated up to 72 hours at 37° C.±6° C. The transduced CD4 or CD8 T cells were assessed for transduction frequency as measured by flow cytometry using a fluorescently-labelled anti-idiotypic antibody specific to the extracellular antigen-binding domain of the CAR. Transduction frequency was calculated as the percentage of CD3+CAR+ cells in the transduced donor cell population (CD4 or CD8 cells).

As shown in Table E1, a high degree of variation in transduction frequency was observed for both CD4 and CD8 cell populations. The transduction frequency of CD4 cells ranged from 35-93% depending on the donor, and the transduction frequency of CD8 cells ranged from 16-92% depending on the donor.

TABLE E1

| | Donor | | Transduction Frequency (% CD3+CAR+) | | | | |
|---|---|---|---|---|---|---|---|
| Cell | samples | Distribution | Range | | | | Standard |
| Population | (n) | Fit | Minimum | Maximum | Mean | Median | Deviation |
| CD4 | 277 | Johnson Sl | 35 | 93 | 67 | 67 | 10.8 |
| CD8 | 267 | Johnson Sl | 16 | 92 | 63 | 65 | 15.4 |

Studies were conducted to determine factors that may impact variance in T cell transduction frequency. Primary CD4+ and CD8+ T cells were selected from isolated PBMCs from a plurality of human donor leukapheresis samples, stimulated with anti-CD3/anti-CD28 paragmagnetic beads and transduced with a lentiviral vector containing nucleic acid encoding a heterologous protein, substantially as described above. Experiments were performed on separate populations of CD4+ and CD8+ T cells as described above, as well as on a population of CD4+ and CD8+ T cells. Factors such as the donor, vector lot, the CD4 and/or CD8 T cell subtype, the process used for transduction, and analytic variability were assessed for association to variance in transduction frequency. As shown in Table E2, these studies demonstrated that the principal source (about 70%) of total variance in T cell transduction frequency was attributable to heterogeneity in the donor T cell population.

TABLE E2

| | | | | | Variance Associated |
|---|---|---|---|---|---|
| T Cell | Healthy | | T Cell Component | Statistical | with Donor Heterogeneity |
| Component | Human Donors | Vector Lots | Lots Manufactured | Test | (%) |
| CD4 & CD8 | 6 | 8 | 70 | Mixed-model | 67* |
| | | | | Restricted | |
| | | | | Maximum | |
| | | | | Likelihood | |
| CD4 | 7 | 7 | 21 | Random-effects | 87** |
| | | | | Restricted | |
| | | | | Maximum | |
| | | | | Likelihood | |
| CD8 | 7 | 7 | 21 | Random-effects | 83** |
| | | | | Restricted | |
| | | | | Maximum | |
| | | | | Likelihood | |

*Study considered the effect of donor, component, process, and analytical variability
**Study considered the effect of donor, process, and analytical variability

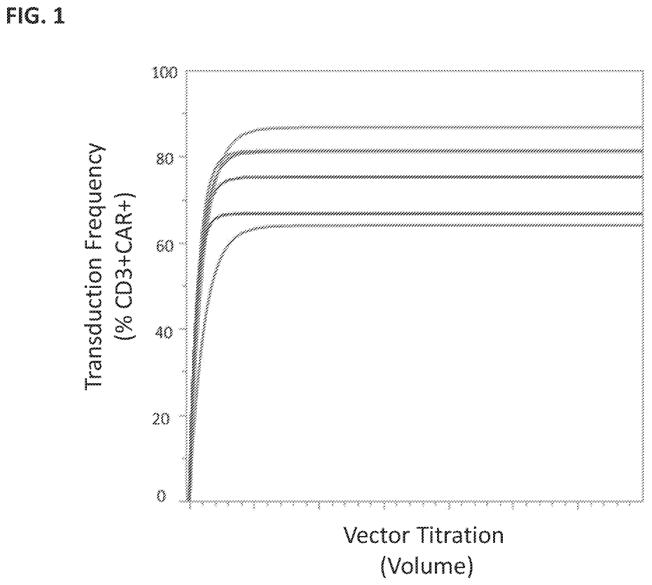

To further assess the contribution of donor versus viral vector on variance of transduction frequency, vector titration experiments were carried out in which the amount of vector was increased by volume titration. CD4 and CD8 T cells from six human donors were isolated, stimulated and transduced substantially as described above. As shown in FIG. 1, variation in the maximum transducible frequency of T cells between donors was observed, and this variation was not overcome by increasing the titration of vector. This demonstrates that there are differences among the population of T cells from different donors that appear to affect transduction frequency.

### Example 2: Assessment of Transduction Frequency Among T Cell Subsets

T cell compositions that included either CD4 cells or CD8 cells were transduced with lentiviral vectors encoding a chimeric antigen receptor (CAR), substantially as described in Example 1.

In one study, CD4+ and CD8+ T cells were separately selected from isolated PBMCs from a leukapheresis sample from 3 healthy donors. Transduction frequency was monitored 24, 48, and 72 hours after inoculation with viral vector, using an anti-idiotyptic antibody as described in Example 1. Cells also were analyzed by flow cytometry for phenotypic characterization based on the differentiation marker CCR7. Less differentiated naïve T cells (Tₙ) and central memory T cells (T_{cm}) are characterized by CCR7 expression (CCR7+), and more differentiated effector T cells (Tₑ) and effector memory T cells (Tₑₘ) are characterized by the absence of CCR7 expression (CCR7−).

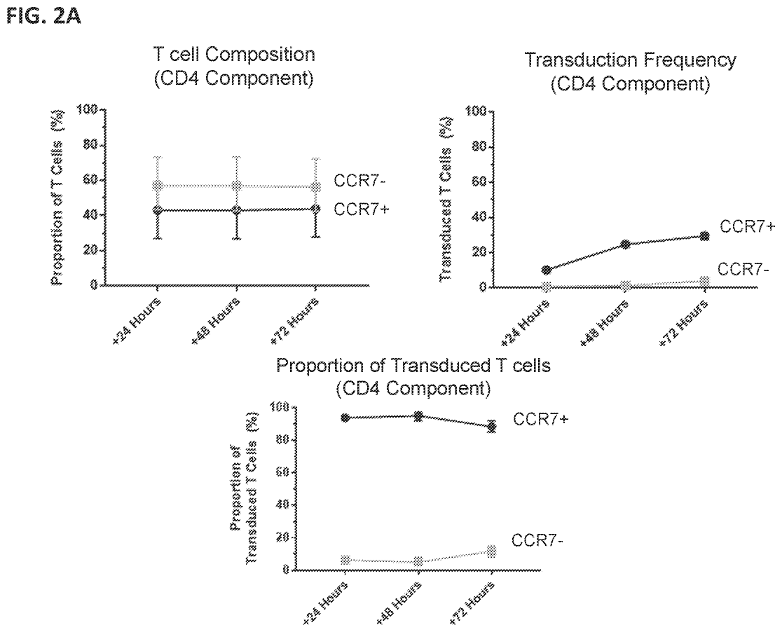

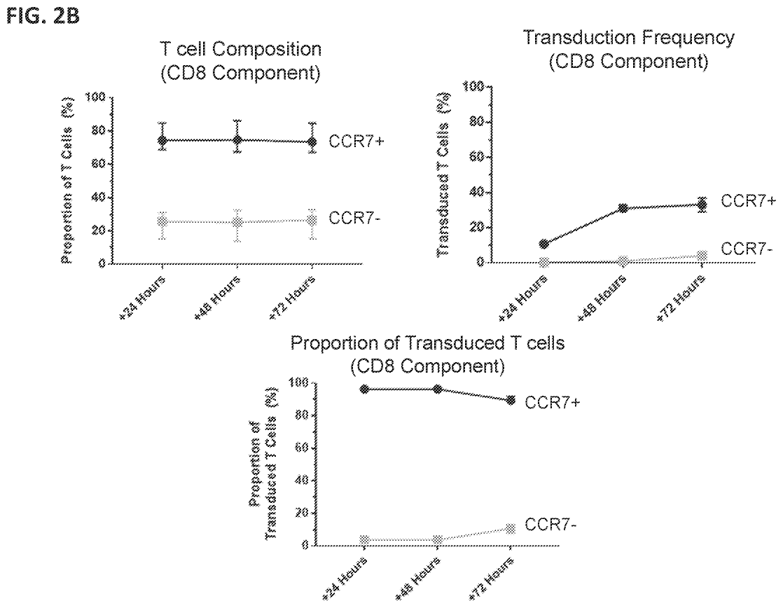

Results are depicted in FIGS. 2A (CD4) and 2B (CD8). In each of FIGS. 2A and 2B, the upper left panel shows the T cell composition prior to activation and transduction (input or selected composition), the upper right panel shows the resulting transduction frequency in those T cell subsets, and the lower panel shows the proportion of the transduced T cells each population represents. The whiskers represent the range of observed data. As shown in the upper right panels of both FIG. 2A and FIG. 2B, phenotypic characterization by flow cytometry demonstrated that less differentiated Tₙ and Tₑₘ cells characterized by CCR7+ expression were transduced at higher frequencies than more differentiated Tₑ and Tₑₘ cells characterized by the absence of CCR7 expression. As shown in the lower panels of FIGS. 2A and 2B, a substantial proportion of transduced CD4 and CD8 T cells were those characterized as being CCR7+. These results demonstrate that CCR7+ T cells have a greater susceptibility to transduction, while CCR7− T cells are less susceptible to transduction, thereby demonstrating that there is an association in both CD4 and CD8 T cells between CCR7 expression and transduction with a viral vector encoding a heterologous protein.

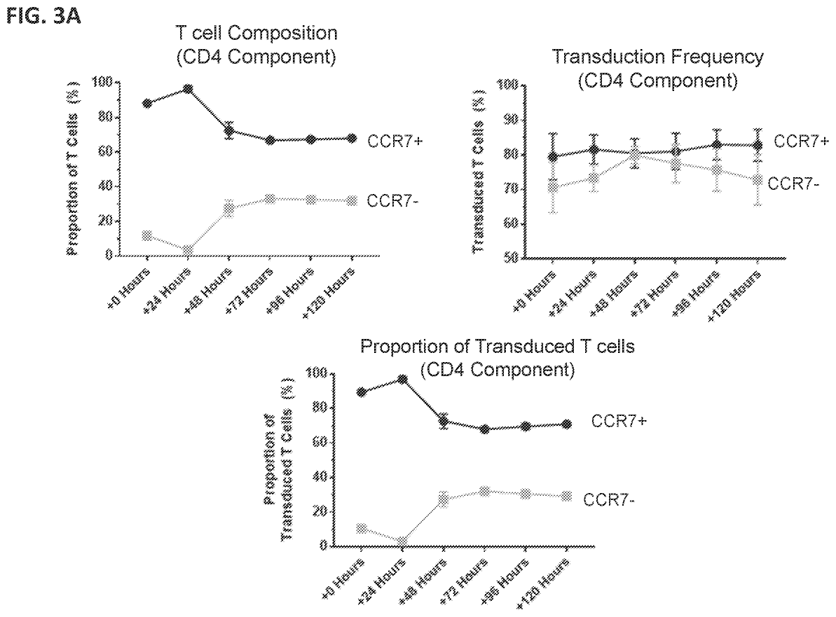

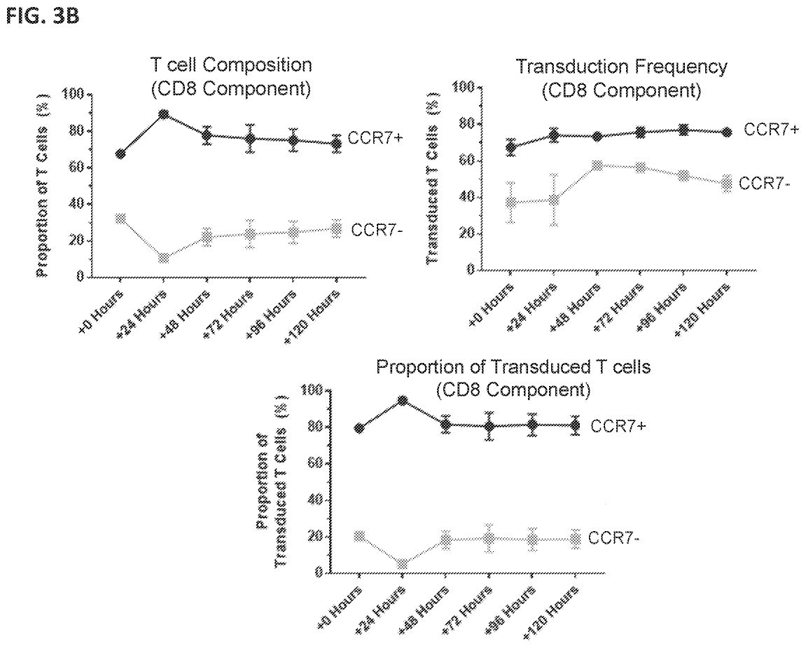

A further study was carried out at larger scale by selection of CD4+ and CD8+ T cells from two healthy human donors, followed by separate stimulation of about 300×10⁶ cells each with anti-CD3/anti-CD28 paramagnetic beads, and transduction via spinoculation with a target volume of lentiviral vector encoding the CAR. Transduction frequency and phenotypic characterization for CCR7 expression were assessed as described above, except at 0, 24, 48, 72, and 120 hours after inoculation with the viral vector. Results are depicted in FIG. 3A (CD4+) and 3B (CD8+). In each figure, the upper left panel shows the T cell composition prior to the activation and transduction, the upper right panel shows the resulting transduction frequency in those T cell subsets, and the lower panel shows the proportion of the transduced T cells each population represents. The whiskers represent the range of observed data. Similar to the smaller scale study described above, a substantial proportion of transduced CD4 and CD8 T cells were those characterized as being CCR7+(FIGS. 3A and 3B, lower panel), and a correlation in both CD4 and CD8 T cells between CCR7 expression and transduction with viral vector was also observed as shown in the upper right panel of FIG. 3A and FIG. 3B, although a stronger correlation was demonstrated in the CD8 T cell composition.

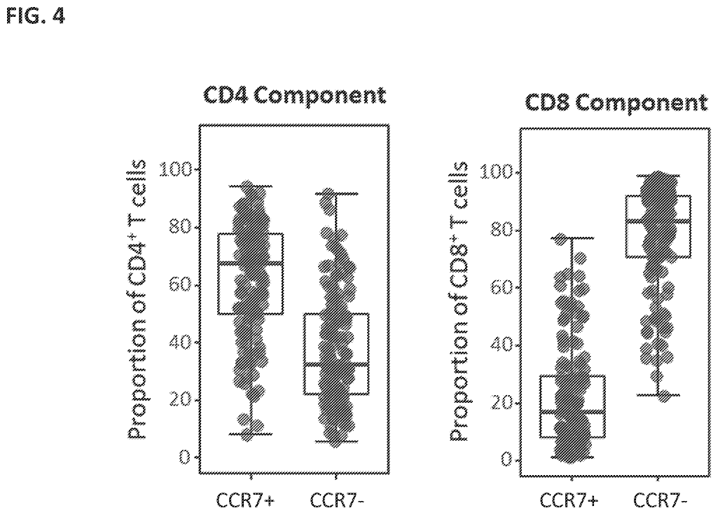

Among subjects described in the study in Example 1 with relapsed/refractory large B-cell lymphoma, the frequency of selected CD4 and CD8 T cell subpopulations that are CCR7+vs CCR7− was assessed by flow cytometry (n=145). Results are depicted in FIG. 4, which demonstrates that CCR7 expression is highly variable in both CD4 and CD8 T cell populations. The boxes as shown in FIG. 4 indicate the interquartile range, while the whiskers indicate the overall range. Since transduction occurs after selection of CD4+ and CD8+ T cells from patient samples, this data reveals that the relative proportion of CCR7+vs CCR7− T cells is highly variable between patient material at the time of transduction. For example, T cell transduction frequency of CD8 T cells, among a groups of subjects with relapsed/refractory large B-cell lymphoma in this study (n=133), was found to be highly correlated with the percentage of CCR7+ T cells immediately prior to the transduction step (n=133; Spearman's rank correlation coefficient, p value <0.0006, Rho 0.302).

Taken together, results from these studies suggests that the variability in transduction frequency between patient samples is primarily the result of differences in the patients' composition of T cell subpopulations, such as the proportion of CCR7+vs CCR7− subpopulations. These results are consistent with a method in which variability in transduction frequency can be minimized or reduced by first selecting CCR7+ T cells from patient samples prior to transduction.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ ID | | |
|---|---|---|
| NO: | Sequence | Description |
| 1 | LQSRPEPTAPPEESFRFGVETTTPPQKQEPIDKELYPLTSLRSLFGN | HIV-1 p6 |
| | DPSSQ | |
| 2 | PMAQVHQGLMPTAPPEDPAVDLLKNYMQLGKQQREKQRESRE | P6 Gag |
| | KPYKEVTEDLLHLNSLFGGDQ | |
| 3 | DXAXXLL | P6 Gag |
| | | packaging |
| | | motif |
| 4 | DPAVDLL | P6 packaging |
| | | motif |
| 5 | DPAVDLLKNY | P6 packaging |
| | | motif |
| 6 | LQSRPEPTAPPEESDPAVDLLTTPPQKQEPIDKELYPLTSLRSLFG | Hybrid or |
| | NDPSSQ | chimeric p6 |
| | | domain |
| 7 | WXXXXXXXXXXXAXXH | Wx4Φx2Φx3 |
| | | AΦxH motif |
| 8 | ESKYGPPCPPCP | spacer |
| | | (IgG4hinge) |
| | | (aa) |
| 9 | GAATCTAAGTACGGACCGCCCTGCCCCCCTTGCCCT | spacer |
| | | (IgG4hinge) |
| | | (nt) |
| 10 | ESKYGPPCPPCPGQPREPQVYTLPPSQEEMTKNQVSLTCLVKGFY | Hinge-CH3 |
| | PSDIAVEWESNGQPENNYKTTPPVLDSDGSFFLYSRLTVDKSRW | spacer |
| | QEGNVFSCSVMHEALHNHYTQKSLSLSLGK | |
| 11 | ESKYGPPCPPCPAPEFLGGPSVFLFPPKPKDTLMISRTPEVTCVVV | Hinge-CH2- |
| | DVSQEDPEVQFNWYVDGVEVHNAKTKPREEQFNSTYRVVSVLT | CH3 spacer |
| | VLHQDWLNGKEYKCKVSNKGLPSSIEKTISKAKGQPREPQVYTL | |
| | PPSQEEMTKNQVSLTCLVKGFYPSDIAVEWESNGQPENNYKTTP | |
| | PVLDSDGSFFLYSRLTVDKSRWQEGNVFSCSVMHEALHNHYTQ | |
| | KSLSLSLGK | |
| 12 | RWPESPKAQASSVPTAQPQAEGSLAKATTAPATTRNTGRGGEEK | IgD-hinge-Fc |
| | KKEKEKEEQEERETKTPECPSHTQPLGVYLLTPAVQDLWLRDKA | |
| | TFTCFVVGSDLKDAHLTWEVAGKVPTGGVEEGLLERHSNGSQS | |
| | QHSRLTLPRSLWNAGTSVTCTLNHPSLPPQRLMALREPAAQAPV | |
| | KLSLNLLASSDPPEAASWLLCEVSGFSPPNILLMWLEDQREVNTS | |
| | GFAPARPPPQPGSTTFWAWSVLRVPAPPSPQPATYTCVVSHEDSR | |
| | TLLNASRSLEVSYVTDH | |
| 13 | LEGGGEGRGSLLTCGDVEENPGPR | T2A |
| 14 | MLLLVTSLLLCELPHPAFLLIPRKVCNGIGIGEFKDSLSINATNIKH | tEGFR |
| | FKNCTSISGDLHILPVAFRGDSFTHTPPLDPQELDILKTVKEITGFL | |
| | LIQAWPENRTDLHAFENLEIIRGRTKQHGQFSLAVVSLNITSLGLR | |
| | SLKEISDGDVIISGNKNLCYANTINWKKLFGTSGQKTKIISNRGEN | |
| | SCKATGQVCHALCSPEGCWGPEPRDCVSCRNVSRGRECVDKCN | |
| | LLEGEPREFVENSECIQCHPECLPQAMNITCTGRGPDNCIQCAHYI | |
| | DGPHCVKTCPAGVMGENNTLVWKYADAGHVCHLCHPNCTYGC | |
| | TGPGLEGCPTNGPKIPSIATGMVGALLLLLVVALGIGLFM | |
| 15 | FWVLVVVGGVLACYSLLVTVAFIIFWV | CD28 (amino |
| | | acids 153-179 |
| | | of Accession |
| | | No. P10747) |
| 16 | IEVMYPPPYLDNEKSNGTIIHVKGKHLCPSPLFPGPSKP | CD28 (amino |
| | FWVLVVVGGVLACYSLLVTVAFIIFWV | acids 114-179 |
| | | of Accession |
| | | No. P10747) |
| 17 | RSKRSRLLHSDYMNMTPRRPGPTRKHYQPYAPPRDFAAYRS | CD28 (amino |
| | | acids 180-220 |
| | | of P10747) |
| 18 | RSKRSRGGHSDYMNMTPRRPGPTRKHYQPYAPPRDFAAYRS | CD28 (LL to |
| | | GG) |
| 19 | KRGRKKLLYIFKQPFMRPVQTTQEEDGCSCRFPEEEEGGCEL | 4-1BB (amino |
| | | acids 214-255 |
| | | of Q07011.1) |
| 20 | RVKFSRSADAPAYQQGQNQLYNELNLGRREEYDVLDKRRGRDP | CD3 zeta |
| | EMGGKPRRKNPQEGLYNELQKDKMAEAYSEIGMKGERRRGKG | |
| | HDGLYQGLSTATKDTYDALHMQALPPR | |
| 21 | RVKFSRSAEPPAYQQGQNQLYNELNLGRREEYDVLDKRRGRDP | CD3 zeta |
| | EMGGKPRRKNPQEGLYNELQKDKMAEAYSEIGMKGERRRGKG | |
| | HDGLYQGLSTATKDTYDALHMQALPPR | |
| 22 | RVKFSRSADAPAYKQGQNQLYNELNLGRREEYDVLDKRRGRDP | CD3 zeta |
| | EMGGKPRRKNPQEGLYNELQKDKMAEAYSEIGMKGERRRGKG | |
| | HDGLYQGLSTATKDTYDALHMQALPPR | |
| 23 | RKVCNGIGIGEFKDSLSINATNIKHFKNCTSISGDLHILPVAFRGDS | tEGFR |
| | FTHTPPLDPQELDILKTVKEITGFLLIQAWPENRTDLHAFENLEIIR | |
| | GRTKQHGQFSLAVVSLNITSLGLRSLKEISDGDVIISGNKNLCYA | |
| | NTINWKKLFGTSGQKTKIISNRGENSCKATGQVCHALCSPEGCW | |
| | GPEPRDCVSCRNVSRGRECVDKCNLLEGEPREFVENSECIQCHPE | |
| | CLPQAMNITCTGRGPDNCIQCAHYIDGPHCVKTCPAGVMGENNT | |
| | LVWKYADAGHVCHLCHPNCTYGCTGPGLEGCPTNGPKIPSIATG | |
| | MVGALLLLLVVALGIGLFM | |
| 24 | EGRGSLLTCGDVEENPGP | T2A |
| 25 | GSGATNFSLLKQAGDVEENPGP | P2A |
| 26 | ATNFSLLKQAGDVEENPGP | P2A |
| 27 | QCTNYALLKLAGDVESNPGP | E2A |
| 28 | VKQTLNFDLLKLAGDVESNPGP | F2A |
| 29 | -PGGG-(SGGGG)5-P- wherein P is proline, G is | Linker |
| | glycine and S is serine | |
| 30 | GSADDAKKDAAKKDGKS | Linker |
| 31 | atgcttctcctggtgacaagccttctgctctgtgagttaccacacccagc | GMCSFR |
| | attcctcctgatccca | alpha chain |
| | | signal |
| | | sequence |
| 32 | MLLLVTSLLLCELPHPAFLLIP | GMCSFR |
| | | alpha chain |
| | | signal |
| | | sequence |
| 33 | MALPVTALLLPLALLLHA | CD8 alpha |
| | | signal peptide |
| 34 | DPSKDSKAQVSAAEAGITGTWYNQLGSTFIVTAGADGALTGTYE | Streptavidin |
| | SAVGNAESRYVLTGRYDSAPATDGSGTALGWTVAWKNNYRNA | Species: |
| | HSATTWSGQYVGGAEARINTQWLLTSGTTEANAWKSTLVGHDT | Streptomyces |
| | FTKVKPSAASIDAAKKAGVNNGNPLDAVQQ | avidinii |
| | | UniProt No. |
| | | P22629 |
| 35 | EAGITGTWYNQLGSTFIVTAGADGALTGTYESAVGNAESRYVLT | Minimal |
| | GRYDSAPATDGSGTALGWTVAWKNNYRNAHSATTWSGQYVG | streptavidin |
| | GAEARINTQWLLTSGTTEANAWKSTLVGHDTFTKVKPSAAS | Species: |
| | | Streptomyces |
| | | avidinii |
| 36 | EAGITGTWYNQLGSTFIVTAGADGALTGTYIGARGNAESRYVLT | Mutein |
| | GRYDSAPATDGSGTALGWTVAWKNNYRNAHSATTWSGQYVG | Streptavidin |
| | GAEARINTQWLLTSGTTEANAWKSTLVGHDTFTKVKPSAAS | Ile44-Gly45- |
| | | Ala-46-Arg47 |
| | | Species: |
| | | Streptomyces |
| | | avidinii |
| 37 | WSHPQFEK | Strep-tag ® II |
| 38 | WSHPQFEKGGGSGGGSGGGSWSHPQFEK | Twin-Strep-tag |
| 39 | WSHPQFEKGGGSGGGSWSHPQFEK | Twin-Strep-tag |
| 40 | WSHPQFEKGGGSGGGSGGSAWSHPQFEK | Twin-Strep-tag |
| 41 | MEAGITGTWYNQLGSTFIVTAGADGALTGTYIGARGNAESRYV | Mutein |
| | LTGRYDSAPATDGSGTALGWTVAWKNNYRNAHSATTWSGQYV | Streptavidin |
| | GGAEARINTQWLLTSGTTEANAWKSTLVGHDTFTKVKPSAAS | Ile44-Gly45- |
| | | Ala-46-Arg47 |
| | | Species: |
| | | Streptomyces |
| | | avidinii |
| 42 | -Trp-Xaa-His-Pro-Gln-Phe-Yaa-Zaa- | Streptavidin- |
| | | binding peptide |
| | | Xaa is any |
| | | amino acid; |
| | | Yaa is Gly or |
| | | Glu |
| | | Zaa is Gly, Lys |
| | | or Arg |
| 43 | Trp-Arg-His-Pro-Gln-Phe-Gly-Gly | Streptavidin |
| | | binding peptide, |
| | | Strep-tag ® |
| 44 | Trp-Ser-His-Pro-Gln-Phe-Glu-Lys-(Xaa)n-Trp-Ser- | Sequential |
| | His-Pro-Gln-Phe-Glu-Lys- | modules of |
| | | streptavidin- |
| | | binding peptide |
| | | Xaa is any |
| | | amino acid; |
| | | n is either 8 or |
| | | 12 |
| 45 | Trp-Ser-His-Pro-Gln-Phe-Glu-Lys-(GlyGlyGlySer)n- | Sequential |
| | Trp-Ser-His-Pro-Gln-Phe-Glu-Lys | modules of |
| | | streptavidin- |
| | | binding peptide |
| | | n is 2 or 3 |
| 46 | SAWSHPQFEKGGGSGGGSGGGSWSHPQFEK | Twin-Strep-tag |
| 47 | SAWSHPQFEKGGGSGGGSGGSAWSHPQFEK | Twin-Strep-tag |
| 48 | DPSKDSKAQVSAAEAGITGTWYNQLGSTFIVTAGADGALTGTYV | Mutein |
| | TARGNAESRYVLTGRYDSAPATDGSGTALGWTVAWKNNYRNA | Streptavidin |
| | HSATTWSGQYVGGAEARINTQWLLTSGTTEANAWKSTLVGHDT | Val44-Thr45- |
| | FTKVKPSAASIDAAKKAGVNNGNPLDAVQQ | Ala46-Arg47 |
| | | Species: |
| | | Streptomyces |
| | | avidinii |
| 49 | EAGITGTWYNQLGSTFIVTAGADGALTGTYVTARGNAESRYVLT | Mutein |
| | GRYDSAPATDGSGTALGWTVAWKNNYRNAHSATTWSGQYVG | Streptavidin |
| | GAEARINTQWLLTSGTTEANAWKSTLVGHDTFTKVKPSAAS | Val44-Thr45- |
| | | Ala46-Arg47 |
| | | Species: |
| | | Streptomyces |
| | | avidinii |
| 50 | MEAGITGTWYNQLGSTFIVTAGADGALTGTYVTARGNAESRYV | Mutein |
| | LTGRYDSAPATDGSGTALGWTVAWKNNYRNAHSATTWSGQYV | Streptavidin |
| | GGAEARINTQWLLTSGTTEANAWKSTLVGHDTFTKVKPSAAS | Val44-Thr45- |
| | | Ala46-Arg47 |
| | | Species: |
| | | Streptomyces |
| | | avidinii |
| 51 | His-Pro-Gln-Phe | Streptavidin- |
| | | binding peptide |
| 52 | Oaa-Xaa-His-Pro-Gln-Phe-Yaa-Zaa | Streptavidin- |
| | | binding peptide |
| | | Oaa is Trp, Lys |
| | | or Arg; Xaa is |
| | | any amino acid; |
| | | Yaa is Gly or |
| | | Glu; Zaa is Gly, |
| | | Lys or Arg |
| 53 | DPSKDSKAQVSAAEAGITGTWYNQLGSTFIVTAGADGALTGTYI | Mutein |
| | GARGNAESRYVLTGRYDSAPATDGSGTALGWTVAWKNNYRNA | Streptavidin |
| | HSATTWSGQYVGGAEARINTQWLLTSGTTEANAWKSTLVGHDT | Ile44-Gly45- |
| | FTKVKPSAASIDAAKKAGVNNGNPLDAVQQ | Ala-46-Arg47 |
| | | Species: |
| | | Streptomyces |
| | | avidinii |
| 54 | EAGITGTWYNQLGSTFIVTAGADGALTGTYVTARGNAESRYVLT | Mutein |
| | GRYDSAPATDGSGTALGWTVAWKNNYRNAHSATTWSGQYVG | Streptavidin |
| | GAEARINTQWLLTSGTTEENAGYSTLVGHDTFTKVKPSAAS | Val44-Thr45- |
| | | Ala46-Arg47 |
| | | and Glu117, |
| | | Gly120, Try121 |
| | | (mutein m1-9) |
| | | Species: |
| | | Streptomyces |
| | | avidinii |
| 55 | DPSKDSKAQVSAAEAGITGTWYNQLGSTFIVTAGADGALTGTY | Mutein |
| | VTARGNAESRYVLTGRYDSAPATDGSGTALGWTVAWKNNYRN | Streptavidin |
| | AHSATTWSGQYVGGAEARINTQWLLTSGTTEENAGYSTLVGHD | Val44-Thr45- |
| | TFTKVKPSAAS | Ala46-Arg47 |
| | | and Glu117, |
| | | Gly120, Try121 |
| | | (mutein m1-9) |
| | | Species: |
| | | Streptomyces |
| | | avidinii |
| 56 | MEAGITGTWYNQLGSTFIVTAGADGALTGTYESAVGNAESRYV | Minimal |
| | LTGRYDSAPATDGSGTALGWTVAWKNNYRNAHSATTWSGQYV | streptavidin |
| | GGAEARINTQWLLTSGTTEANAWKSTLVGHDTFTKVKPSAAS | Species: |
| | | Streptomyces |
| | | avidinii |
| 57 | RASQDISKYLN | CDR L1 |
| 58 | SRLHSGV | CDR L2 |
| 59 | GNTLPYTFG | CDR L3 |
| 60 | DYGVS | CDR H1 |
| 61 | VIWGSETTYYNSALKS | CDR H2 |
| 62 | YAMDYWG | CDR H3 |
| 63 | EVKLQESGPGLVAPSQSLSVTCTVSGVSLPDYGVSWIRQPPRKGL | VH |
| | EWLGVIWGSETTYYNSALKSRLTIIKDNSKSQVFLKMNSLQTDD | |
| | TAIYYCAKHYYYGGSYAMDYWGQGTSVTVSS | |
| 64 | DIQMTQTTSSLSASLGDRVTISCRASQDISKYLNWYQQKPDGTV | VL |
| | KLLIYHTSRLHSGVPSRFSGSGSGTDYSLTISNLEQEDIATYFCQQ | |
| | GNTLPYTFGGGTKLEIT | |
| 65 | DIQMTQTTSSLSASLGDRVTISCRASQDISKYLNWYQQKPDGTV | scFv |
| | KLLIYHTSRLHSGVPSRFSGSGSGTDYSLTISNLEQEDIATYFCQQ | |
| | GNTLPYTFGGGTKLEITGSTSGSGKPGSGEGSTKGEVKLQESGPG | |
| | LVAPSQSLSVTCTVSGVSLPDYGVSWIRQPPRKGLEWLGVIWGS | |
| | ETTYYNSALKSRLTIIKDNSKSQVFLKMNSLQTDDTAIYYCAKHY | |
| | YYGGSYAMDYWGQGTSVTVSS | |
| 66 | KASQNVGTNVA | CDR L1 |
| 67 | SATYRNS | CDR L2 |
| 68 | QQYNRYPYT | CDR L3 |
| 69 | SYWMN | CDR H1 |
| 70 | QIYPGDGDTNYNGKFKG | CDR H2 |
| 71 | KTISSVVDFYFDY | CDR H3 |
| 72 | EVKLQQSGAELVRPGSSVKISCKASGYAFSSYWMNWVKQRPGQ | VH |
| | GLEWIGQIYPGDGDTNYNGKFKGQATLTADKSSSTAYMQLSGLT | |
| | SEDSAVYFCARKTISSVVDFYFDYWGQGTTVTVSS | |
| 73 | DIELTQSPKFMSTSVGDRVSVTCKASQNVGTNVAWYQQKPGQS | VL |
| | PKPLIYSATYRNSGVPDRFTGSGSGTDFTLTITNVQSKDLADYFC | |
| | QQYNRYPYTSGGGTKLEIKR | |
| 74 | GGGGSGGGGSGGGGS | Linker |
| 75 | EVKLQQSGAELVRPGSSVKISCKASGYAFSSYWMNWVKQRPGQ | scFv |
| | GLEWIGQIYPGDGDTNYNGKFKGQATLTADKSSSTAYMQLSGLT | |
| | SEDSAVYFCARKTISSVVDFYFDYWGQGTTVTVSSGGGGSGGGG | |
| | SGGGGSDIELTQSPKFMSTSVGDRVSVTCKASQNVGTNVAWYQ | |
| | QKPGQSPKPLIYSATYRNSGVPDRFTGSGSGTDFTLTITNVQSKD | |
| | LADYFCQQYNRYPYTSGGGTKLEIKR | |
| 76 | HYYYGGSYAMDY | HC-CDR3 |
| 77 | HTSRLHS | LC-CDR2 |
| 78 | QQGNTLPYT | LC-CDR3 |
| 79 | gacatccagatgacccagaccacctccagcctgagcgccagcctgggcgaccgggtgaccatcagc | Sequence |
| | tgccgggccagccaggacatcagcaagtacctgaactggtatcagcagaagcccgacggcaccgtc | encoding scFv |
| | aagctgctgatctaccacaccagccggctgcacagcggcgtgcccagccggtttagcggcagcggct | |
| | ccggcaccgactacagcctgaccatctccaacctggaacaggaagatatcgccacctacttttgccag | |
| | cagggcaacacactgccctacacctttggcggcggaacaaagctggaaatcaccggcagcacctcc | |
| | ggcagcggcaagcctggcagcggcgagggcagcaccaagggcgaggtgaagctgcaggaaagc | |
| | ggccctggcctggtggcccccagccagagcctgagcgtgacctgcaccgtgagcggcgtgagcctg | |
| | cccgactacggcgtgagctggatccggcagccccccaggaagggcctggaatggctgggcgtgatc | |
| | tggggGagGgagaGGaGGtaGtaGaaGagGgGGGtgaagagGGggGtgaGGatGatGaaggaGaaG | |
| | agcaagagccaggtgttcctgaagatgaacagcctgcagaccgacgacaccgccatctactactgcg | |
| | ccaagcactactactacggcggcagctacgccatggactactggggccagggcaccagcgtgaccg | |
| | tgagcagc | |
| 80 | GSTSGSGKPGSGEGSTKG | Linker |

What is claimed:

1. A method for increasing transduction frequency of primary T cells, the method comprising:
(a) selecting primary T cells that are positive for surface expression of CCR7 from a biological sample comprising a population of primary T cells, thereby generating an input population enriched in CCR7+ primary T cells;
(b) incubating the input population under stimulatory conditions, thereby generating a stimulated composition, wherein said stimulating conditions comprise the presence of a stimulatory reagent capable of activating one or more intracellular signaling domains of one or more components of a TCR complex and/or one or more intracellular signaling domains of one or more costimulatory molecules; and
(c) incubating a viral vector particle comprising a heterologous polynucleotide encoding a recombinant protein with T cells of the stimulated composition, thereby generating a population of transduced cells.

2. A method for increasing transduction frequency of primary T cells, the method comprising:
(a) selecting primary T cells that are positive for surface expression of CCR7 from a biological sample comprising a population of primary T cells, thereby generating an input population enriched in CCR7+ primary T cells; and
(b) incubating a viral vector particle comprising a heterologous polynucleotide encoding a recombinant protein with T cells of the input population of cells, thereby generating a population of transduced cells.

3. A method for increasing transduction frequency of primary T cells, the method comprising:
(a) incubating an input population of primary T cells enriched in CCR7+ T cells under stimulatory conditions, thereby generating a stimulated composition, wherein said stimulating conditions comprise the presence of a stimulatory reagent capable of activating one or more intracellular signaling domains of one or more components of a TCR complex and/or one or more intracellular signaling domains of one or more costimulatory molecules; and
(b) incubating a viral vector particle comprising a heterologous polynucleotide encoding a recombinant protein with T cells of the stimulated composition, thereby generating a population of transduced cells.

4. A method for increasing transduction frequency of primary T cells, the method comprising incubating a viral vector particle comprising a heterologous polynucleotide encoding a recombinant protein with T cells of an input population of primary T cells enriched in CCR7+ T cells, thereby generating a population of transduced cells.

5. The method of any one of claims 1-4, wherein at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the input population are CCR7+ primary T cells.

6. The method of any one of claims 1-5, wherein at least 90%, at least 91%, at least 92%, at least 93%, at least 94%, at least 95%, at least 96%, at least 97%, at least 98%, at least 99%, or 100% of the input population are CCR7+ primary T cells.

7. The method of any one of claims 1-6, wherein at least 95%, at least 96%, at least 97%, at least 98%, at least 99%, or 100% of the input population are CCR7+ primary T cells.

8. The method of any one of claims 1, 2, and 5-7, wherein the biological sample is a blood sample.

9. The method of any one of claims 1, 2, and 5-7, wherein the biological sample is a leukapheresis sample.

10. The method of any one of claims 1-9, wherein the T cells are unfractionated T cells, are enriched or isolated CD3+ T cells, are enriched or isolated CD4+ T cells, or are enriched or isolated CD8+ T cells.

11. The method of any one of claims 1-10, wherein the input population comprises at least 80%, at least 85%, at least 90%, or at least 95% cells that are CD4+ T cells or CD8+ T cells.

12. The method of any one of claims 1-11, wherein the input population comprises at least 80%, at least 85%, at least 90%, or at least 95% cells that are CD4+ T cells.

13. The method of any one of claims 1-11, wherein the input population comprises at least 80%, at least 85%, at least 90%, or at least 95% cells that are CD8+ T cells.

14. The method of any one of claims 1-11, wherein the input population comprises at least 80%, at least 85%, at least 90%, or at least 95% cells that are CD4+ T cells and CD8+ T cells.

15. The method of claim 14, wherein the ratio of the CD4+ T cells to the CD8+ T cells is or is about 1:1, 1:2, 2:1, 1:3, or 3:1.

16. The method of any one of claims 1-10, wherein the input population comprises at least 80%, at least 85%, at least 90%, or at least 95% cells that are CD3+ T cells.

17. The method of any one of claims 1-16, wherein the input population comprises between 100×10⁶ and 500×10⁶ total T cells.

18. The method of any one of claims 1-17, wherein the input population comprises between 200×10⁶ and 400×10⁶ total T cells, optionally at or about 300×10⁶ total T cells.

19. The method of claim 17 or claim 18, wherein the total T cells are viable T cells.

20. The method of any one of claims 1, 3, and 519 wherein at least 10%, at least 20%, at least 30%, at least 40%, at least 50%, or at least 60% of the cells of the stimulated composition:
(i) express a surface marker selected from the group consisting of HLA-DR, CD25, CD69, CD71, CD40L, and 4-1BB;
(ii) comprise intracellular expression of a cytokine selected from the group consisting of IL-2, IFN-gamma, and TNF-alpha;
(iii) are in the G1 or later phase of the cell cycle; and/or
(iv) are capable of proliferating.

21. The method of any one of claims 1, 3, and 5-20 wherein the stimulatory reagent comprises a primary agent that specifically binds to a member of a TCR complex, optionally that specifically binds to CD3.

22. The method of claim 21, wherein the stimulatory reagent further comprises a secondary agent that specifically binds to a T cell costimulatory molecule, optionally wherein the costimulatory molecule is selected from CD28, CD137 (4-1-BB), OX40, or ICOS.

23. The method of claim 21 or claim 22, wherein the primary and/or secondary agents comprise an antibody, optionally wherein the stimulatory reagent comprises incubation with an anti-CD3 antibody and an anti-CD28 antibody, or an antigen-binding fragment thereof.

24. The method of any one of claims 21-23, wherein the primary agent and/or secondary agent are present on the surface of a solid support.

25. The method of claim 24, wherein the solid support is or comprises a bead.

26. The method of any of claims 21-23, wherein the primary agent and secondary agent are reversibly bound on the surface of an oligomeric particle reagent comprising a plurality of streptavidin or streptavidin mutein molecules.

27. The method of claim 26, wherein each of the plurality of the streptavidin or streptavidin mutein molecules comprise the amino acid sequence of Val44-Thr⁴⁵-Ala⁴⁶-Arg⁴⁷ or Ile⁴⁴-Gly⁴⁵-Ala⁴⁶-Arg⁴⁷ at sequence positions corresponding to positions 44 to 47 with reference to positions in streptavidin in the sequence of amino acids set forth in SEQ ID NO: 34.

28. The method of claim 26, wherein each of the plurality of the streptavidin or streptavidin mutein molecules are streptavidin mutein molecules, and wherein each of the plurality of the streptavidin mutein molecules is or comprises:
a) the sequence of amino acids set forth in any of SEQ ID NOS: 36, 41, 48-50, or 53-55;
b) a sequence of amino acids that exhibit at least 85%, 86%, 87%, 88%, 89%, 89%, 90%, 91%, 92%, 93%, 94%, 95%, 96%, 97%, 98%, or 99% or more sequence identity to any of SEQ ID NOS: 36, 41, 48-50, or 53-55 and contain the amino acid sequence corresponding to Val44-Thr45-Ala46-Arg47 or Ile44-Gly45-Ala46-Arg47 and/or reversibly bind to biotin, a biotin analog or a streptavidin-binding peptide; or
c) a functional fragment of a) or b) that reversibly binds to biotin, a biotin analog, or a streptavidin-binding peptide, optionally wherein each of the plurality of the streptavidin mutein molecules is or comprises the amino acid sequence set forth in SEQ ID NO:36 or SEQ ID NO:41.

29. The method of any one of claims 1-28, wherein the population of transduced cells comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% cells that express the recombinant protein.

30. The method of any one of claims 1-29, wherein the population of transduced cells comprises at least 55%, at least 60%, at least 65%, at least 70%, at least 75%, at least 80%, at least 85%, at least 90%, or at least 95% cells that express the recombinant protein.

31. The method of any one of claims 1-30, wherein the percentage of cells in the population of transduced cells expressing the recombinant protein is at least 0.5-fold, at least 1-fold, at least 1.5-fold, or at least 2-fold greater as compared to a cell composition that was not enriched for CCR7+ primary T cells through a selection step.

32. The method of any of claims 1-31, wherein the incubating the viral vector particle comprises a step of spinoculating the viral vector particles with the input population or the stimulated composition.

33. The method of claim 32, wherein spinoculating comprises rotating, in an internal cavity of a centrifugal chamber, the viral vector particles and the input population or stimulated composition, wherein the rotation is at a relative centrifugal force at an internal surface of the side wall of the cavity that is:
between or between about 500 g and 2500 g, 500 g and 2000 g, 500 g and 1600 g, 500 g an 1000 g, 600 g and 1600 g, 600 g and 1000 g, 1000 g and 2000 g, or 1000 g and 1600 g, each inclusive; or
at least or at least about 600 g, 800 g, 1000 g, 1200 g, 1600 g, or 2000 g.

34. The method of claim 32 or claim 33, wherein spinoculating is for a time that is:
greater than or about 5 minutes, greater than or about 10 minutes, greater than or about 15 minutes, greater than or about 20 minutes, greater than or about 30 minutes, greater than or about 45 minutes, greater than or about 60 minutes, greater than or about 90 minutes, or greater than or about 120 minutes; or
between or between about 5 minutes and 60 minutes, 10 minutes and 60 minutes, 15 minutes and 60 minutes, 15 minutes and 45 minutes, 30 minutes and 60 minutes, or 45 minutes and 60 minutes, each inclusive.

35. The method of any of claims 1, 3, and 5-34, further comprising during at least a portion of the incubating contacting the stimulated composition and/or viral vector particles with a transduction adjuvant.

36. The method of any of claims 2, 4-34, further comprising, during at least a portion of the incubating, contacting the input population and/or viral vector particles with a transduction adjuvant.

37. The method of claim 35 or claim 36, wherein the contacting is carried out prior to, concomitant with, or after the spinoculating the viral vector particles with the input population or the stimulated composition.

38. The method of any of claims 1-37, wherein at least a portion of the incubation of the viral vector particle is carried out at or about 37° C.±2° C.

39. The method of any of claims 1-38, wherein at least a portion of the incubation of the viral vector particle is carried out after the spinoculation.

40. The method of claim 38 or claim 39, wherein the at least a portion of the incubation of the viral vector particle is carried out for no more than or no more than about 2 hours, 4 hours, 12 hours, 18 hours, 24 hours, 30 hours, 36 hours, 48 hours, 60 hours, or 72 hours.

41. The method of any of claims 38-40, wherein the at least a portion of the incubation of the viral vector particle is carried out for or for about 24 hours.

42. The method of any of claims 1-41, wherein the total duration of the incubation of the viral vector particle is for no more than 12 hours, 24 hours, 36 hours, 48 hours, or 72 hours.

43. The method of any of claims 1-42, wherein the viral vector particle is a lentiviral vector particle.

44. The method of claim 43, wherein the lentiviral vector particle is replication defective.

45. The method of any of claims 1-44, wherein the viral vector particle is pseudotyped with a viral envelope glycoprotein.

46. The method of claim 45, wherein the viral envelope glycoprotein is VSV-G.

47. The method of any of claims 1-46, wherein the viral vector particle is incubated at a multiplicity of infection of less than or less than about 20.0 or less than or less than about 10.0.

48. The method of any of claims 1-47, wherein:
the viral vector particle is incubated at a multiplicity of infection from or from about 1.0 IU/cell to 10 IU/cell, or 2.0 U/cell to 5.0 IU/cell; or
the viral vector particle is incubated at a multiplicity of infection of at least or at least about 1.6 IU/cell, 1.8 IU/cell, 2.0 IU/cell, 2.4 IU/cell, 2.8 IU/cell, 3.2 IU/cell, 3.6 IU/cell, 4.0 IU/cell, 5.0 IU/cell, 6.0 IU/cell, 7.0 IU/cell, 8.0 IU/cell, 9.0 IU/cell, or 10.0 IU/cell.

49. The method of any of claims 1, 3, and 5-48 wherein the stimulated composition comprises at least at or about at least or about 50×10⁶ cells, 100×10⁶ cells, or 200×10⁶ cells.

50. The method of any of claims 1, 3, and 5-49, wherein the stimulated composition comprises between at or about 50×10⁶ cells and at or about 300×10⁶ cells, inclusive, optionally between at or about 100×10⁶ cells and at or about 200×10⁶ cells, inclusive.

51. The method of any of claims 2 and 4-48, wherein the input population comprises at least at or about at least or about 50×10⁶ cells, 100×10⁶ cells, or 200×10⁶ cells.

52. The method of any of claims 2 and 4-49, wherein the input population comprises between at or about 50×10⁶ cells and at or about 300×10⁶ cells, inclusive, optionally between at or about 100×10⁶ cells and at or about 200×10⁶ cells, inclusive.

53. The method of any of claims 1-52, wherein the T cells incubated with the viral particle comprises at least at or about at least or about 50×10⁶ cells, 100×10⁶ cells, or 200×10⁶ cells.

54. The method of any of claims 1-53, wherein the T cells incubated with the viral particle comprises between at or about 50×10⁶ cells and at or about 300×10⁶ cells, inclusive, optionally between at or about 100×10⁶ cells and at or about 200×10⁶ cells, inclusive.

55. The method of any of claims 1-54, wherein the recombinant protein is an antigen receptor.

56. The method of claim 55, wherein the antigen receptor is a transgenic T cell receptor (TCR).

57. The method of claim 55, wherein the antigen receptor is a chimeric antigen receptor (CAR).

58. The method of claim 57, wherein the CAR comprises an extracellular antigen-recognition domain that specifically binds to a target antigen, an intracellular signaling domain comprising an ITAM, and a transmembrane domain linking the extracellular domain and the intracellular signaling domain.

59. The method of claim 58, wherein the intracellular signaling domain comprises an intracellular domain of a CD3-zeta (CD3ζ) chain.

60. The method of claim 58 or claim 59, wherein the transmembrane domain comprises a transmembrane portion of CD28.

61. The method of any of claims 58-60, wherein the intracellular signaling domain further comprises an intracellular signaling domain of a T cell costimulatory molecule.

62. The method of claim 61, wherein the T cell costimulatory molecule is selected from the group consisting of CD28 and 41BB.

63. The method of any of claims 55-62, wherein the antigen receptor specifically binds to an antigen associated with a disease or condition or specifically binds to a universal tag.

64. The method of claim 63, wherein the disease or condition is a cancer, an autoimmune disease or disorder, or an infectious disease.

65. The method of any of claims 1-64, wherein the population of transduced cells comprises T cells transduced with the heterologous polynucleotide.

66. The method of claim 65, wherein at least 20%, at least 25%, at least 30%, at least 35%, at least 40%, at least 45%, at least 50%, at least 55%, at least 60%, at least 65%, at least 70%, at least 75%, at least 80%, or at least 85% of the T cells in the population of transduced cells are transduced with the heterologous polynucleotide.

67. The method of claim 65 or claim 66, wherein at least 55%, at least 60%, at least 65%, at least 70%, at least 75%, at least 80%, or at least 85% of the T cells in the population of transduced cells are transduced with the heterologous polynucleotide.

68. The method of any one of claims 65-67, wherein at least 95%, at least 96%, at least 97%, at least 98%, or at least 99% of the T cells transduced with the heterologous polynucleotide are CCR7+.

69. The method of claim 65 or claim 66, further comprising recovering or isolating from the population of transduced cells the transduced T cells produced by the method.

70. The method of any one of claims 1-69, wherein, among a plurality of populations of transduced cells, the percentage of T cells in the population of transduced cells that are transduced with the heterologous polynucleotide varies by 30% or less, 25% or less, 20% or less, 15% or less, or 10% or less.

71. The method of any of claims 1-70 that is carried out in vitro or ex vivo.

72. A composition comprising a population of transduced cells produced by the method of any one of claims 1-71.

73. The composition of claim 72, further comprising a cyropreservant.
